(19) United States
(12) Patent Application Publication
(10) Pub. No.: US 2016/0038575 A1
(43) Pub. Date: Feb. 11, 2016

(54) COMPOSITIONS AND METHODS FOR IMMUNE TOLERANCE INDUCTION TO FACTOR VIII REPLACEMENT THERAPIES IN SUBJECTS WITH HEMOPHILIA A

(71) Applicants: HAPLOMICS, INC., Atlanta, GA (US); THE REGENTS OF THE UNIVERSITY OF CALIFORNIA, Oakland, CA (US); DEPARTMENT OF VETERANS AFFAIRS, Washington, DC (US)

(72) Inventors: Tommy E. HOWARD, Redondo Beach, CA (US); Vincent LA TERZA, Atlanta, GA (US)

(21) Appl. No.: 14/776,709

(22) PCT Filed: Mar. 17, 2014
(86) PCT No.: PCT/US14/30314

§ 371 Date: Sep. 14, 2015

Related U.S. Application Data
(60) Provisional application No. 61/792,102, filed on Mar. 15, 2013.

(51) Int. Cl. A61K 39/00; A61K 47/48; A61K 47/34

(52) U.S. Cl. CPC A61K 39/001; A61K 47/34; A61K 47/482; A61K 2039/6093; A61K 2039/577

(57) ABSTRACT

This disclosure relates to tolerance inducing peptide (TIP) derived from the amino acid reference locus (AARL) within a FVIII replacement product (FVIIIrp) based on the differences between the expression product of a subject's F8 gene (sFVIII) and the FVIIIrp to provide tolerance induction before, during, and/or after a FVIII replacement therapy in a subject suffering from Hemophila A. Methods of deriving, making, and using the TIP are also disclosed. In some embodiments, the TIP is associated with a nanoparticle, e.g., PLGA or PLGA-PEMA nanoparticle.

### CROSS REFERENCE TO RELATED APPLICATIONS

This application claims priority to U.S. provisional patent application Ser. No. 61/792,102, filed on Mar. 15, 2013 to Howard et al., entitled “Compositions and Methods for Immune Tolerance Induction to Factor VIII Replacement Therapies in Subjects with Hemophilia A,” incorporated herein by reference.

### GOVERNMENT RIGHTS

Development of the inventions described herein was at least partially funded with government support through NIH/NHLBI Grant RC2 HL 101851 and the U.S. government has certain rights in the inventions.

### FIELD OF THE INVENTION

This invention is in the area of compositions for and improved methods of inducing tolerance or reducing or minimizing an immune response to a FVIII replacement product in a subject suffering from hemophilia who will receive, is receiving, or has received the FVIII replacement product by administering tolerance inducing peptides, or sets of peptides, derived from the amino acid differences between the subject's endogenous FVIII and the FVIII replacement product.

### BACKGROUND OF THE INVENTION

Hemophilia A (HA) is a congenital bleeding disorder caused by loss-of-function mutations in the X-linked Factor VIII (FVIII) gene, F8. FVIII is an essential cofactor in the blood coagulation pathway. Defects within the F8 gene affect about one in 5000 males. The levels of functional FVIII in circulation determine the severity of the disease, with plasma levels 5-25% of normal being mild, 1-5% being moderate, and <1% being severe. As such, only a small amount of circulating protein is necessary to provide protection from spontaneous bleeding episodes.

Patients with HA are treated with FVIII replacement therapies, i.e., infusions of either extracted and pooled human plasma-derived (pd)FVIII and/or recombinant (r)FVIII replacement products. Currently available rFVIII replacement products include the commercially available Kogenate® (Bayer) and Helixate® (ZLB Behring), Recombinate® (Baxter) and Advate® (Baxter), and the B-domain deleted Refacto® (Pfizer) and Xyntha® (Pfizer). pdFVIII is largely derived from pooled blood collections in Europe and the United States. In many cases, treatment with FVIII replacements provides efficient management of this chronic disease. In approximately 25-30% of cases, however, this treatment leads to the patients developing anti-FVIII neutralizing antibodies, termed inhibitors, which reduces the effectiveness of the FVIII replacement or, in the worst case, renders the replacement ineffective (Lacroix-Desmazes et al., Pathophysiology of inhibitors to FVIII in patients with haemophilia A. Haemophilia 2002: 8: 273-9). In hemophilia A patients of African-American descent, inhibitors occur in approximately 50% of individuals following FVIII replacement therapy. The development of inhibitors leads to the neutralization of the pro-coagulant function of the FVIIII replacement or enhances its removal from the plasma (Lacroix-Desmazes et al., Dynamics of factor VIII interactions determine its immunologic fate in hemophilia A. Blood 2008; 112: 240-9). The development of FVIII inhibitors significantly increases the morbidity and lowers the quality of life for patients who develop inhibitors, and represents the greatest limitation to successful FVIII replacement therapy (Darby et al., The incidence of factor VIII and factor IX inhibitors in the hemophilia population of the UK and their effect on subsequent mortality, 1977-99. J Throm Haemost 2004: 2: 1047-54; Ehrenforth et al., Incidence of development of factor VIII and factor IX inhibitors in hemophiliacs. Lancet 1992; 339: 594-8; Lusher et al., Recombinant factor VIII for the treatment of previously untreated patients with hemophilia A. Safety, efficacy, and development of inhibitors. Kogenate Previously Untreated Patient Study Group. NEJM 1993; 328: 453-9).

Inhibitors can be transient or low-responding (i.e., a peak Bethesda titer <5 BU/mL) or high-responding (i.e., a peak Bethesda titer >5 BU/mL). In low-responding inhibitor patients, bleeding episodes may be managed by administering increased FVIII replacement dosages. In patients with high-responding inhibitors, bleeding episodes are generally managed by administering by-passing agents such as recombinant activated factor VII and activated prothrombin complex concentrates (Paisley et al., The management of inhibitors in haemophilia A: introduction and systematic review of current practice. Haemophilia 2003; 9; 405-17; Bentorp et al., Inhibitor treatment in haemophilias A and B: summary statement for the 2006 international consensus conference. Haemophilia 2006; 12 (Suppl. 6): 1-7). For example, FEIBA® is a plasma derived bypassing agent that includes activated FX and prothrombin. NovoSeven®, a recombinant bypassing agent (rFVIIa), is also used to control bleeding in high responder patients. While its mechanism of action is still debated, what is known is that NovoSeven®'s bypassing activity and ability to provide hemostasis in bleeding HA patients with FVIII inhibitors requires infusion at markedly supra-physiologic levels (Shibeko et al., Unifying the mechanism of recombinant FVIIa action: dose dependence is regulated differently by tissue factor and phospholipids. Blood 2012; 120: 891-9). Regardless of the underlying mechanism, its effects are variable across patients leading to high dosing protocols. The licensed dosing regimen for NovoSeven® is 90 μg/kg given up to every 2-hours (Shapiro et al., Prospective, randomised trial of two doses of rFVIIa (NovoSeven) in haemophilia patients with inhibitors undergoing surgery. Thromb Haemost 1998; 80: 773-8). A major shortcoming of bypassing agents is the lack of quantitative clinical laboratory assays necessary to accurately monitor procoagulant activity to guide therapy. The challenge presented by this opacity is exacerbated by the absence of an optimal dose or dosing schedule for bypassing agents (Acharya et al., Management of factor VIII inhibitors. Best Pract Res Clin Haematol 2006; 19: 51-66). Furthermore, bypassing agents can and have been reported to induce thromboembolic events.

Restoring FVIII replacement treatment efficacy is highly desirable to improve outcomes for patients who have developed FVIII inhibitors. Currently, strategies to induce immune tolerance to replacement FVIII therapies in patients who have developed inhibitors consists of regular and prolonged administration of FVIII replacement concentrates (See Coppola et al., Optimizing management of immune tolerance induction in patients with severe haemophilia A and inhibitors: towards evidence-based approaches. British J Haem 2010; 150: 515-28). Both high-dose and low-dose protocols have been attempted with mixed results, and each protocol can be demanding on patients and extremely expensive, as continuous infusions of FVIII replacement products for various time periods are generally employed. For example, in Europe, immune tolerance induction treatment of at least 6 to 12 months is suggested (Astermark et al., Current European practice in immune tolerance induction therapy in patients with haemophilia and inhibitors. Haemophilia 2006; 12: 363-71). In clinical practice, these induction strategies are often continued beyond 33 months, as some patients may require longer duration of treatment for achieving tolerance (Kurth et al., Immune tolerance therapy utilizing factor VIII/von Willebrand factor concentrate in haemophilia A patients with high titre factor VIII inhibitors. Haemophilia 2008; 14: 50-55). Importantly, utilizing these strategies results in a significant increased risk in the number of bleeding episodes at all stages of tolerance induction. It fails in 20% to 40% of patients and is challenging to implement, especially in children given the continuous need for vein access for administration of the infusions (Coppola et al., Optimizing management of immune tolerance induction in patients with severe haemophilia A and inhibitors: towards evidence-based approaches. British J Haem 2010; 150: 515-28).

Although immune tolerance induction therapies to FVIII replacement products have been around for many years, there is very little experimental data elaborating the mechanism of action of repetitive, long term FVIII infusion mediated tolerance. While it has been suggested that T cell immune exhaustion (over stimulation and subsequent T cell anergy or apoptosis) plays a role in achieving tolerance utilizing these strategies, there is no experimental evidence to support this hypothesis (Waters et al., The molecular mechanisms of immunomodulation and tolerance induction to factor VIII. J Throm Haemost 2009; 7: 1446-56). Several studies investigating the mechanisms of tolerance induction have shown that high FVIII levels inhibit memory B cell differentiation, and that tolerance induction can lead to the generation of anti-idiotypic Abs in cured patients (Gilles et al., Neutralizing anti-idiotypic antibodies to factor VIII inhibitors after desensitization in patients with hemophilia A. J Clin Invest 1996; 97: 1382-8; Hausl et al., High-dose factor VIII inhibits factor VIII-specific memory B cells in hemophilia A with factor VIII inhibitors. Blood 2005; 106: 3415-22; Hausl et al., Preventing re-stimulation of memory B cells in hemophilia A: a potential new strategy for the treatment of antibody dependent immune disorders. Blood 2004; 104: 115-22; Gilles et al., In vivo neutralization of a C2 domain-specific human anti-Factor VIII inhibitor by an anti-idiotypic antibody. Blood 2004; 103: 2617-23). As previously mentioned, however, tolerance induction through this route requires the continuous use of FVIII replacement product, is expensive, can take years to work, and occurs after the patient has already developed inhibitors.

Given the drawbacks of current therapeutic options to manage inhibitor patients and the limitations, arduous nature and expense of immune tolerance protocols, there is a need for strategies that achieve FVIII replacement therapy tolerance before, during, and/or after a patient develops inhibitors. Furthermore, there is a need to develop immune tolerance strategies able to impart tolerance to FVIII replacement products that do not require daily, long term FVIII replacement product infusions.

### SUMMARY OF THE INVENTION

Methods and compositions are provided for the minimization of an undesired immune response and/or induction of immune tolerance to a FVIII replacement product in subjects having hemophilia A and who will be administered, are being administered, or have been administered a FVIII replacement product (FVIIIrp). In particular, the present invention provides for the identification of amino acid differences between the expression product of a subject's F8 gene (sFVIII) and the FVIIIrp including the recombinant FVIII replacement product (rFVIIIrp) or plasma-derived FVIII replacement product (pdFVIIIrp) used to restore FVIII activity and coagulation in the subject, and the creation of overlapping sets of tolerogenic peptides (termed herein as tolerance inducing peptides (TIPs)) based on such amino acid differences that are administered to the subject in order to minimize an undesired immune response and/or induce tolerance to the FVIIIrp, for example, by preventing, minimizing, reducing, or eliminating inhibitor formation against the FVIIIrp In particular embodiments, the FVIIIrp is a rFVIIIrp.

The amino acid differences between the sFVIII and FVIIIrp may fall within T-cell epitopes that are capable of inducing an undesired immune response to the FVIIIrp when the FVIIIrp is administered to the subject. These differences may include an amino acid residue difference at a single locus or an amino acid residue difference at more than one locus, for example in the case of a missense mutation or the presence of nsSNPs, or both. These differences may include the presence of amino acid residues in the FVIIIrp at one or more loci that are not present in the sFVIII due to a deletion in the subject's F8 gene. Or, in the case of F8 intron 22 inversion mutations—the most common mutation in severe FVIII deficiency—the differences may include amino acid residues that arise due to the proteolytic liberation of a T cell epitope which occurs in the FVIIIrp, which does not occur with the subject's endogenous FVIII or is not made available so as to react with the subject's immune system by a proteolytic event involving the subject's endogenous FVIII. For subjects receiving rFVIIIrp lacking a B-domain (B-domain deleted rFVIIIrp or “BDD-rFVIIIrp”), these differences may include short linker peptides connecting the A2 and A3 domains of the BDD-rFVIIIrp that result in potential T-cell epitopes due to a novel protein sequence that is not present in subject's endogenous FVIII proteins.

Amino acid residue difference between the sFVIII and FVIIIrp are positioned or mapped within specific loci in the FVIIIrp, wherein the differing FVIIIrp amino acids—individually termed the amino acid reference locus (AARL)—serves as a reference point or points for the preparation of a set or sets of tolerizing peptides—termed tolerizing amino acids (“TAAs”) or tolerance inducing peptides (“TIPs”) that may incorporate T-cell epitopes capable of inducing immune tolerance of, or the prevention, reduction, or elimination of inhibitor development by the subject to the FVIIIrp. Each TIP within a set includes a FVIIIrp amino acid residing at a reference locus, and a TIP set includes between about 9 to 21 separate peptides of between 9 to 21 amino acids in length, wherein the number of peptides in a TIP set is directly correlated with the length of the TIP (i.e., a TIP set containing TIPs each having 9 amino acids in length will contain 9 peptides; a TIP set containing TIPs each having 10 amino acids in length will contain 10 peptides, etc.).

A method of designing the amino acid sequence residue required to derive a TIP or TIP set is generally as follows. The first peptide of each TIP set has as its first amino acid position the first amino acid residue of a reference locus of the FVIIIrp, while the remaining amino acid residues are identical to the downstream amino acids in the FVIIIrp across the length of the TIP. If only a single amino acid residue difference exists at the locus (for example in the case of a missense mutation or nsSNP), then the reference locus will consist of a single amino acid residue. If the differences encompass more than one contiguous amino acid residue (for example in the case of some deletions), then the first differing amino acid residue in the FVIIIrp will serve as the reference locus. For example, if the TIP is 9 amino acids in length, the first amino acid in the first peptide will be the first amino acid of the reference locus, and the remaining 8 amino acid residues will be the 8 loci residues of the FVIIIrp immediately downstream from the reference locus (as determined from amino acid position 1 to 2332 in the wt FVIII protein). The second peptide of each TIP has as its second amino acid position the reference locus, with the first amino acid position being the first amino acid residue in the FVIIIrp immediately upstream from the reference locus, and the remaining 7 amino acid residues being the 7 loci residues of the FVIIIrp immediately downstream from the reference locus. As such, for each successive TIP in the TIP set, the reference locus is shifted one amino acid position downstream, and the first amino acid reflects a shift from the preceding peptide of one amino acid upstream in the FVIIIrp. Accordingly, the last TIP of the set—in the preceding example, the ninth peptide—will have the reference locus in the last amino acid residue position, and be preceded by upstream amino acid residues—in the preceding example, the 8 residues of the FVIIIrp immediately upstream of the reference locus. The same method described above can be generally used to create TIP sets of varying peptide sizes, wherein the reference locus in each successive peptide in the set is shifted one position downstream and the first amino acid position in each successive peptide is shifted one residue upstream from the first amino acid position in the preceding peptide, until the reference locus occupies the last amino acid position in the last peptide of the set.

Following the method of generating sets of TIPs as described above, a set of TIPs will correspond with a contiguous portion of the FVIIIrp across 2X−1 amino acids, where X is the length of the peptides contained in the set. For example, as described in the preceding example, a TIP set containing 9 peptides, each being 9 amino acids in length, will as a set overlap with 17 contiguous amino acids of the FVIIIrp. Furthermore, the contiguous FVIIIrp amino acid sequence overlapped by the TIPs will include X−1 amino acid residues upstream and X−1 amino acid residues downstream from the first amino acid of the reference locus within the FVIIIrp, wherein X is the length of the peptides contained in the set. For example, a set of 9 peptides of 9 amino acids in length will overlap with 8 amino acids upstream and 8 amino acids downstream from the first amino acid of the reference locus within the FVIIIrp. This general process will be applicable to the generation of TIP sets for most identified amino acid differences, with a few exceptions, for example in the derivation of TIP sets to a few BDD-rFVIIIrp synthetic linker as described further herein.

The present invention provides for the administration of an effective amount of one or more of the overlapping TIPs from each TIP set in order to prevent or limit the development of, or minimize, reduce, or eliminate the existence of, inhibitors to the specific FVIIIrp. In certain embodiments, a set of TIPs comprising at least 9 peptides of 9 amino acids in length each are administered. Without wishing to be bound by any particular theory, it is believed that peptides that have the potential to be proteolysis products and be presented by MHC molecules in a subject's antigen presenting cells (APCs) can be immunogenic and initiate the development of inhibitors. By administering an effective amount of specific TIPs in a tolerizing fashion, the present invention provides for a targeted tolerance induction and/or minimized or reduced immune response strategy to potential T cell epitopes in the FVIIIrp that are implemented prior to the development of inhibitors, or, if inhibitors have already developed, in a more tolerable and less expensive approach than current tolerance inducing protocols which require repetitive, long term infusion of FVIIIrp. The administration of the TIPs and TIP sets described herein may result in a reduction of measurable Bethesda titer units to a FVIIIrp in a subject that already has inhibitors to a FVIIIrp. For example, the reduction of measurable Bethesda titer units is at least 10%, i.e., 20%, 30%, 40%, 50%, 60%, 70%, 80%, 90%, 95%, or 99.9%.

By determining a subject's endogenous FVIII amino acid sequence and comparing it to the known amino acid sequence of a rFVIIIrp, differences between the sFVIII and the rFVIIIrp amino acid sequences are identified, and sets of peptides comprising TIPs are created, wherein one or more TIPs from each set, or, in some embodiments the entire TIP set, are administered to induce tolerance in the subject that will be, is, or has been receiving the rFVIIIrp. Differences between a sFVIII and a rFVIIIrp can result from, for example, mis sense mutations in the subject's F8 gene, nonsynonymous single-nucleotide polymorphisms (nsSNPs) or haplotypic variations between the sFVIII and rFVIIIrp, deletions, inversions, for example intron 1 or 22 inversions, administration of rFVIIIrp with synthetic linker sequences, for example BDD-rFVIIIrp, and the like, or combinations thereof.

The reference locus of a TIP may positionally correlate with an amino acid substitution in the sFVIII caused by a missense mutation in the subject's F8 gene. In one embodiment, sets of TIPs containing at least 9 amino acids and including a reference locus are derived from the TIPs described in Tables 2-87. In one embodiment, at least one TIP from a TIP set described in Tables 2-87 are administered to minimize an undesired immune response to a FVIIIrp. In one embodiment, at least the first 9 peptides comprising the first 9 amino acids of a TIP set described in Tables 2-87 are administered. In one embodiment, at least the first 15 peptides comprising the first 15 amino acids of a TIP set described in Tables 2-87 are administered to minimize an undesired immune response. In one embodiment, at least the first 17 peptides comprising the first 17 amino acids of a TIP set described in Tables 2-87 are administered to induce tolerance. In one embodiment, a TIP set described in Tables 2-87 is administered to minimize an undesired immune response.

The currently available rFVIIIrp products are derived from H1 and or H2 wild-type haplotypes. Furthermore, pdFVIIIrp is largely derived from donors having the H1 haplotype. In one embodiment, the reference locus of the TIP positionally correlates with a nsSNP or haplotypic variation contained in the sFVIII. In one embodiment, a set of TIPs containing at least 9 amino acids and including a reference locus are derived from the TIPs described in Tables 88-101. In one embodiment, at least one TIP from a TIP set described in Tables 88-101 are administered to minimize an undesired immune response. In one embodiment, at least the first 9 peptides comprising the first 9 amino acids of a TIP set described in Tables 88-101 are administered to minimize an undesired immune response. In one embodiment, at least the first 15 peptides comprising the first 15 amino acids of a TIP set described in Tables 88-101 are administered to minimize an undesired immune response. In one embodiment, at least the first 17 peptides comprising the first 17 amino acids of a TIP set described in Tables 88-101 are administered to minimize an undesired immune response. In one embodiment, a TIP set described in Tables 88-101 are administered to minimize an undesired immune response.

Generally, subject's with the F8 intron 22 inversion express the entire FVIII protein intracellularly, albeit on two separate polypeptides. Importantly, another gene, F8_{B}, is also generally expressed in both normal and HA subjects. The expression product of the F8_{B} gene, FVIII_{B}, has sequence identity with a portion of the C1 domain and the entire C2 domain of FVIII. The presence of this FVIII_{B} polypeptide is important from a tolerance standpoint as it serves as a source for any T cells epitope or B cell epitopes needed to support processes that occur in the thymus (T cell clonal deletion) and spleen (B cell anergy) to achieve central tolerance. The expression product of F8_{I22I} starts at residue 1 and ends at residue 2124. The polypeptide expressed by the F8_{B} begins at residue 2125 and ends at residue 2332. Accordingly subjects having the F8_{I22I} have the requisite FVIII material to yield one or more FVIII peptides ending at or before residue 2124, the last amino acid encoded by exon 22, or beginning at or after residue 2125, the first amino acid encoded by exon 23. Any potential T cell epitope within such a peptide would be expected to be recognized as a self-antigen and not be immunogenic in the subject. Peptides spanning the junction between residues 2124 and 2125, if proteolyzed from a FVIIIrp and presented by MHC class II molecules, however, would be “foreign” and potentially harbor immunogenic T cell epitopes in an F8_{I22I} subject. Because of this, all subjects having F8_{I22I} mutations have similar reference loci across residues 2124Val and 2125Met with respect to all currently available rFVIIIrp, and a set of TIPs containing at least 9 amino acids and including this MV rFVIIIrp locus are derived from the TIPs described in Table 102. In one embodiment, at least one TIP from the TIP set described in Table 102 are administered to minimize an undesired immune response. In one embodiment, at least the first 9 peptides comprising the first 9 amino acids of a TIP set described in Table 102 are administered to minimize an undesired immune response. In one embodiment, at least the first 15 peptides comprising the first 15 amino acids of a TIP set described in Table 102 are administered to minimize an undesired immune response. In one embodiment, at least the first 17 peptides comprising the first 17 amino acids of a TIP set described in Table 102 are administered to minimize an undesired immune response. In one embodiment, a TIP set described in Table 102 are administered to minimize an undesired immune response.

In one embodiment, the reference locus of a TIP positionally correlates with a differing amino acid sequence within the rFVIIIrp caused by the removal of the B-domain from a BDD-rFVIIIrp. In certain BDD-rFVIIIrp, a deletion of 894 internal codons and splicing codons 762 and 1657 creates a FVIII product containing 1438 amino acids. The BDD-rFVIIIrp contains a synthetic junctional 14-peptide sequence SFS-QNPPVLKRHQR formed by covalent attachment of the three N-terminal most residues of the B-domain, S⁷⁴¹F⁷⁴²S⁷⁴³, to the 11 C-terminal-most residues Q¹⁶³⁸N¹⁶³⁹P¹⁶⁴⁰P¹⁶⁴¹V¹⁶⁴²L¹⁶⁴³K¹⁶⁴⁴R¹⁶⁴⁵H¹⁶⁴⁶Q¹⁶⁴⁷R¹⁶⁴⁸. This synthetic linker creates 11 unique peptides across a 15 amino acid sequence within the BDD-rFVIIIrp, which have potential immunogenicity. In one embodiment, a set of TIPs containing at least 9 amino acids and including a reference locus are derived from the TIPs described in Table 103. In one embodiment, at least one TIP from a TIP set described in Table 103 can administered to minimize an undesired immune response. In one embodiment, at least the first 5 peptides comprising the first 9 amino acids of the TIP set described in Table 103 are administered to minimize an undesired immune response. In one embodiment, a TIP set described in Table 103 are administered to minimize an undesired immune response.

Once TIP sets are identified, one or more of the peptides from the TIP set are manufactured and administered to the subject in a tolerizing fashion. In one embodiment, peptides of the TIP set are analyzed to identify immunodominant T-cell epitopes and at least one or more of the peptides containing immunodominant T-cell epitopes are administered. In some aspects, the immunodominant T-cell epitope is an epitope known to bind with high affinity to one or more MHC class II molecules, such binding being a prerequisite to stimulate an immune response against rFVIIIrp by presentation on MHC-class II. In one embodiment, at least one TIP from at least one TIP set is administered. In one embodiment, more than one TIP from at least one TIP set is administered.

In one aspect of the invention, compositions and methods directed to TIP sets comprising at least 9 peptides, and in the case of BDD-rFVIIIrp differences at least 5 peptides, containing at least 9 amino acids and including a reference locus are provided. By administering a set of TIPs associated with a potential T cell epitope in the rFVIIIrp, as opposed to less than all identified such TIPs, the requirement that immunodominant T-cell epitopes be analyzed according to MHC-II binding affinity correlated with a subject's HLA profile is by-passed. Furthermore, by administering a set of TIPs, the potential that a MHC-II binding epitope, if it exists, will be administered from the set is enhanced, as all identified peptides are administered. In one embodiment, the entire set of TIPs directed to a reference locus is administered. In one embodiment, the entire set of TIPs for each identified reference locus is administered. One of ordinary skill in the art will appreciate that particularly in the context of administration to a rFVIIIrp naive subject or to a subject that is free of anti-FVIII inhibitors, if a subject's MHC-II repertoire is not competent to present a set of TIPs, the risk of an untoward immune response being triggered by potentially immunogenic T cell epitopes residing in the rFVIIIrp is minimal, since the subject's MHC-II will not be competent to present them either.

A sFVIII and a FVIIIrp may have more than one amino acid difference across their respective sequences. For example, the subject may have both a mis sense mutation and a different FVIII haplotype than that of the FVIIIrp, rendering more than one differences between the sequences, or other differences due to other causative combinations of amino acid differences. In such as case, it is contemplated that a set of TIPs directed to each reference locus may be developed, and TIPs from one or more of the TIP sets may be administered. In one embodiment, at least one TIP from at least one TIP set is administered. In one embodiment, at least one TIP from two or more TIP sets is administered. In one embodiment, at least one TIP directed to each identified reference locus is administered. In one embodiment, the entire set of TIPs for each identified reference locus is administered.

TIPs directed to reference loci may be administered before, during, or after exposure to a FVIIIrp. In one embodiment, at least one TIP from a TIP set, or alternatively the entire TIP set, is administered prophylactically to a subject that has not previously been treated with the FVIIIrp. In one embodiment, at least one TIP from a TIP set, or alternatively the entire TIP set, is administered to a subject who is currently undergoing treatment with the FVIIIrp, but has not yet developed inhibitors to the specific FVIIIrp. In one embodiment, at least one TIP from a TIP set, or alternatively the entire TIP set, is administered to a subject concomitantly with the FVIIIrp. In one embodiment, at least one TIP from a TIP set, or alternatively the entire TIP set, is administered to a subject who has previously been treated with the FVIIIrp. In one embodiment, at least one TIP from a TIP set, or alternatively the entire TIP set, is administered as a tolerizing maintenance dose to a subject who has previously been tolerized to an FVIIIrp.

In some embodiments, the TIPs described herein are combined with immune suppressive compounds, or administered in conjunction with immune suppressive compounds, that are capable of inducing antigen-specific adaptive regulatory T cells, including but not limited to IL-10, rapamycin (or other limus compounds, including but not limited to biolimus A9, everolimus, tacrolimus, and zotarolimus), and/or TGF-β, and/or combinations thereof.

In some embodiments, the TIPs described herein are administered as an alternative to, an adjunct to, or in addition to, other FVIII tolerance induction therapy. For example, in one embodiment, at least one TIP from a TIP set, or alternatively the entire TIP set, is administered to a subject who has developed inhibitors to the FVIIIrp and is undergoing standard tolerance induction therapy, for example, a repetitive long term FVIIIrp infusion.

TIPs for administration are from about 9 amino acids to about 22 amino acids in length. The length of each TIP within each TIP set is generally the same, that is, all peptides within the TIP set will be the same amino acid length. The length of peptides between different TIP sets are the same length, or, in an alternative embodiment, different in length. For example, a subject with, for example, two separate amino acid differences between his FVIII protein and the FVIIIrp, are administered tolerogenic peptides from two TIP sets, wherein the first TIP set is directed to a first reference locus wherein each peptide in the set is, for example, 16 amino acids in length, and a second TIP set is directed to a second reference locus the length of the peptides within a particular TIP set is between about 9 amino acids and 22 amino acids. In one embodiment, the length of the peptides within a particular TIP set is at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, or at least 22 amino acids. In one embodiment, the length of the peptides within a particular TIP set is 9 amino acids, 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, or 22 amino acids. In one embodiment, the length of the TIPs within the TIP set is 9 amino acids. In one embodiment, the length of the TIPs within the TIP set is 15 amino acids. In one embodiment, the length of the TIPs within the TIP set is between 17 and 21 amino acids. In one embodiment, the length of the TIPs within the TIP set is 17 amino acids. In one embodiment, the length of the TIPs within the TIP set is 18 amino acids. In one embodiment, the length of the TIPs within the TIP set is 19 amino acids. In one embodiment, the length of the TIPs within the TIP set is 20 amino acids. In one embodiment, the length of the TIPs within the TIP set is 21 amino acids.

At least one TIP, or alternatively a TIP set, from more than one TIP set targeting the same reference locus can be administered. For example, a first TIP set may comprise peptides of, for example, 9 amino acids, and a second TIP set targeting the same reference locus may comprise peptides of, for example, 16 amino acids, wherein both TIP sets are directed to the same reference locus.

Generally, the length of the peptides within each set of TIPs will determine the number of peptides contained within each set. For example, if the length of the peptides within a set is 21 amino acids in length, then 21 peptides will be contained in that particular TIP set.

The present invention includes delivering to a subject at least one TIP directed to a reference locus in a tolerizing fashion. In one embodiment, the entire TIP set is delivered to the subject. As described herein, TIPs are delivered in such a way so as minimize, reduce, or eliminate the subject's immune response to a FVIIIrp epitope that includes a reference locus. In one embodiment, administration of the TIPs described herein induces T-cell tolerance. In one embodiment, the administration of the TIPs described herein induces T-cell anergy. In one embodiment, the administration of the TIPs described herein induces abortive T-cell activation. In one embodiment, the TIPs of the present invention are administered to target the natural mechanisms for clearing apoptotic debris. In one embodiment, the TIPs are delivered in such a way so as to be taken up by marginal zone macrophages expressing the macrophage receptor protein MARCO. In one embodiment, the TIPs are delivered in such a way so as to be taken up by immature dendritic cells. In one embodiment, the TIPs are solubilized. In one embodiment, the TIPs are delivered intravenously.

The TIPs described herein are administered to a subject in association with a carrier. In one embodiment, the TIP is coupled to a carrier to form a TIP-carrier complex. In one embodiment, the TIP is covalently coupled to a carrier molecule. In one embodiment, the TIP is covalently coupled to a carrier molecule using 1-ethyl-3-(3-dimethylaminopropyl)carbodiimide (ECDI). In one embodiment, the carrier is selected from the group consisting of an isologous leukocyte and a micro- or nano-particle. In one embodiment, the micro- or nano-particle is a biodegradable micro- or nano-particle. In one embodiment, the biodegradable micro- or nano-particle is a poly(lactide-co-glycolide)(PLGA) micro- or nano-particle. In one embodiment, the biodegradable micro- or nano-particle is a PLGA particle modified with PEMA (poly[ethylene-co-maleic acid]) as a surfactant to form a PLGA-PEMA micro- or nano-particle. In one embodiment, the PLGA micro- or nano-particle or PLGA-PEMA particle has a size of between about 10 nm to about 5000 nm. In one embodiment, the PLGA or PLGA-PEMA micro- or nano-particle has a size between about 200 nm to about 1000 nm. In one embodiment, the PLGA, PLGA-PEMA micro- or nano-particle has a size of about 400 nm to about 600 nm, and in particular embodiments, about 500 nm. In one embodiment, the micro- or nano-particle is a polystyrene micro- or nano-particle. In one embodiment, the polystyrene micro- or nano-particle has a size of between about 10 nm to about 5000 nm. In one embodiment, the polystyrene micro- or nano-particle has a size between about 200 nm to about 1000 nm. In one embodiment, the polystyrene micro- or nanoparticle has a size of about 400 nm to about 600 nm, and in particular embodiments, about 500 nm.

In one embodiment, the TIPs described herein are coupled to a PLGA, PLGA-PEMA, PLA, or polystyrene (PS) micro- or nano-particle that is about 200 nm to about 1000 nm in size, about 400 nm to about 600 nm, and in particular about 500 nm, using ECDI.

In one aspect of the present invention, compositions are provided herein comprising at least one or more TIPs from a TIP set useful for administering to a HA subject in order to minimize an undesired immune response to a FVIIIrp. In one embodiment, composition are provided comprising at least one TIP from a TIP set, wherein the TIP is a result of a missense mutation, an non-synonymous SNP or haplotypic variation, a deletion, an inversion, or a synthetic linker peptide contained in a FVIIIrp, for example a BDD-rFVIIIrp. In one embodiment, compositions are provided comprising at least one TIP of at least 9 amino acids in length, wherein the peptide encompasses a reference locus, identified in the TIP sets identified in Tables 2-103. In one embodiment, a composition comprising at least one TIP of at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, or at least 21 amino acids, and including a reference locus is provided, wherein the reference locus results from a missense mutation, a non-synonymous SNP or haplotypic variation, a deletion, an inversion, or a synthetic linker peptide contained in a rFVIIIrp, for example, a BDD-rFVIIIrp. In one embodiment, a composition comprising at least one TIP of at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, or at least 21 amino acids, and including a reference locus is provided, wherein the peptide is derived from the peptide sequences described in Tables 2-103.

Compositions comprising at least one TIP comprising at least 9 amino acids comprised from the TIPs in Tables 2-103 are provided. Compositions comprising at least one TIP set comprising at least 9 peptides comprised from the TIP sets in Tables 2-102 are provided. Compositions comprising at least one TIP set comprising at least 5 peptides comprised from the TIP set in Tables 103 are provided.

The TIPs described herein can be coupled to a carrier. In one embodiment, the peptide is covalently couple to a carrier molecule. In one embodiment, the peptide is covalently coupled to a microparticle. In one embodiment, the TIP is covalently coupled to a microparticle using ECDI. In one embodiment, the microparticle is a PLGA, PLGA-PEMA, PLA, or polystyrene bead of between about 200 nm and about 1000 nm. In one embodiment, the microparticle is about 500 nm. In one embodiment, the composition includes at least 1, 2, 3, 4, 5, 6, 7, 8, 9, 10, 11, 12, 13, 14, 15, 16, 17, 18, 19, 20, or 21 or more peptides. In one embodiment, the composition includes TIPs from more than one TIP set. Alternatively, the TIPs described herein are incorporated into, or encapsulated by, a carrier.

In one aspect of the present invention, compositions are provided herein comprising at least one TIP set of peptides useful for administering to a HA subject in order to minimize or reduce an undesired immune response to a FVIIIrp. In one embodiment, compositions are provided comprising at least one TIP set, wherein the TIP within the set is a result of a missense mutation, a non-synonymous SNP or haplotypic variation, an inversion, or a synthetic linker in a FVIIIrp. In one embodiment, compositions are provided comprising at least one TIP set identified in Tables 2-103. In one embodiment, a composition comprising at least one TIP set of at least 9 peptides, at least 10 peptides, at least 11 peptides, at least 12 peptides, at least 13 peptides, at least 14 peptides, at least 15 peptides, at least 16 peptides, at least 17 peptides, at least 18 peptides, at least 19 peptides, at least 20 peptides, or at least 21 peptides is provided, wherein the reference locus within the set is a result of a mis sense mutation, an non-synonymous SNP or haplotypic variation, or an inversion. In one embodiment, a composition comprising at least one TIP set of at least 9 peptides, at least 10 peptides, at least 11 peptides, at least 12 peptides, at least 13 peptides, at least 14 peptides, at least 15 peptides, at least 16 peptides, at least 17 peptides, at least 18 peptides, at least 19 peptides, at least 20 peptides, or at least 21 peptides is provided, wherein the TIP set is described in Tables 2-103. In one embodiment, the peptides of the TIP set are coupled to at least one carrier. In one embodiment, the peptides of the TIP set are coupled to one or, alternatively, more than one carrier. In one embodiment, the peptides of the TIP set are covalently coupled to a carrier. In one embodiment, the peptides of the TIP set are covalently coupled to a micro- or nano-particle. In one embodiment, the peptides of the TIP set are covalently coupled to a micro- or nano-particle using ECDI. In one embodiment, the micro- or nano-particle is a PLGA, PLGA-PEMA, PLA, or polystyrene bead of between about 200 nm and about 1000 nm, between about 400 nm and about 600 nm, and, more particularly, around about 500 nm. In one embodiment, the micro- or nano-particle is about 500 nm. In one embodiment, the composition comprises at least one TIP set. In one embodiment, the composition comprises two or more TIP sets. In one embodiment, the composition comprises a set of peptides for each reference locus identified.

In one embodiment, the TIPs or TIP sets described herein are administered prophylactically to a subject that has not previously been treated with an FVIIIrp. In one embodiment, the TIPs or TIP sets described herein are administered to a subject who is currently undergoing treatment with an FVIIIrp, but has not yet developed inhibitors to the specific FVIIIrp. In one embodiment, the TIPs or TIP sets described herein are administered to a subject concomitantly with the administration of an FVIIIrp. In one embodiment, at least one TIP from a TIP set, or alternatively the entire TIP set, is administered to a subject who has previously been treated with the FVIIIrp. In one embodiment, the TIPs or TIP sets described herein are administered as a tolerizing maintenance dose to a subject who has previously been tolerized to an FVIIIrp. In one embodiment, the TIPs or TIP sets described herein are administered to a subject who has developed inhibitors to the FVIIIrp and has previously undergone standard tolerance induction therapy, for example, a repetitive long-term FVIIIrp infusion. In one embodiment, the TIPs or TIP sets described herein are administered to a subject who has developed inhibitors to an FVIIIrp and is currently undergoing standard tolerance induction therapy, for example, a repetitive long-term FVIIIrp infusion. In one embodiment, the TIPs or TIP sets described herein are administered to a subject who has developed inhibitors to the FVIIIrp and is concomitantly initiating standard tolerance induction therapy, for example, a repetitive long-term FVIIIrp infusion.

The present invention includes at least the following features:

1) methods for the minimization of an undesired immune response and/or induction of immune tolerance to a FVIII replacement product, including but not limited to a rFVIIIrp, in a subject suffering from hemophilia A including determining the amino acid differences between the subject's FVIII and the FVIIIrp to be administered, being administered, or having been administered to the subject, identifying one or more reference locus within the FVIIIrp, wherein the reference locus correlates with an amino acid difference between the sFVIII and the FVIIIrp, identifying a set of TIPs between 9 and 21 peptides, wherein the length of each peptide correlates with the number of peptides in the set, wherein each TIP includes the reference locus and is identical to a contiguous amino acid sequence within the FVIIIrp, and administering at least one or more TIPs, or a at least one or more sets of TIPs, to a subject;

2) Compositions and methods for creating TIPs for use in minimizing an undesired immune response and/or inducing immune tolerance to a FVIII replacement product, including but not limited to a rFVIIIrp, in a subject suffering from hemophilia A including determining the amino acid differences between the subject's FVIII and the FVIIIrp to be administered, being administered, or having been administered to the subject, identifying one or more reference locus within the FVIIIrp, wherein the reference locus correlates with an amino acid difference between the sFVIII and the FVIIIrp, creating a set of TIPs comprising between 9 and 21 peptides, wherein the TIP corresponds with a contiguous amino acid sequence within the FVIIIrp, wherein the length of the peptide is directly correlated with the number of peptides in the set, wherein each peptide in the set includes the reference locus, wherein the first peptide of the set comprises a reference locus at its first amino acid position, the second peptide of the set comprises a reference locus at its second amino acid position, and each successive peptide in the set comprises a reference locus at an amino acid position frame-shifted one position downstream from the reference locus position of the preceding peptide, and wherein the last peptide of the set has a reference locus in its last amino acid position;

3) Compositions, and methods for minimizing an undesired immune response and/or inducing immune tolerance to a FVIII replacement product, including but not limited to a rFVIIIrp, in a subject suffering from hemophilia A using such compositions, including one or more peptides of at least 9 amino acids long generated from the TIPs identified in Tables 2-103; and,

4) Compositions, and methods for minimizing an undesired immune response and/or inducing immune tolerance to a FVIII replacement product, including but not limited to rFVIIIrp, in a subject suffering from hemophilia A using such compositions, including one or more sets of TIPs, wherein each TIP set comprises at least 9 peptides selected from at least the first 9 peptides of one of the TIP sets identified in Tables 2-103.

### BRIEF DESCRIPTION OF THE DRAWINGS

FIG. 1: Shown are FVII haplotypic variants, distribution in the black and white population, and development of inhibitors associated with replacement FVIII treatment.

FIG. 2: Schematic of a reference locus identified between an exemplary sFVIII amino acid sequence and a rFVIIIrp, and a TIP set of 9 TIPs, each incorporating the reference locus, of 9 amino acids in length.

FIG. 3: Schematic of illustrative TIP sets of between 9 amino acids in length to 21 amino acids in length derived from an exemplary reference locus.

### DETAILED DESCRIPTION OF THE INVENTION

### Definitions

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In some embodiments, the subject is a mammal such as a primate, for example, a human.

“Amount effective” and “effective amount” in the context of a composition or dosage form for administration to a subject refers to an amount of the composition or dosage form that produces one or more desired immune tolerizing responses in the subject, for example, the generation of a tolerogenic immune response to a rFVIIIrp immunogenic epitope resulting in the prevention, reduction, or elimination of an immunogenic response to a rFVIIIrp, for example prevention, reduction, or elimination of inhibitors to the rFVIIIrp. Therefore, in some embodiments, an amount effective is any amount of a composition provided herein that produces one or more of these desired immune responses. The amount are one that a clinician believe to have a clinical benefit for a subject in need of rFVIIIrp antigen-specific tolerization.

Effective amount can involve only reducing the level of an undesired immune response, although in some embodiments, it involves preventing an undesired immune response altogether. Effective amount can also involve delaying the occurrence of an undesired immune response. An amount that is effective can also be an amount of a composition provided herein that produces a desired therapeutic endpoint or a desired therapeutic result. Effective amount result in a tolerogenic immune response in a subject to a rFVIIIrp. The achievement of any of the foregoing are monitored by routine methods.

In some embodiments of any of the compositions and methods provided, the effective amount is one in which the desired minimization or reduction of an undesired immune response persists in the subject for at least 1 week, at least 2 weeks, at least 1 month, at least 2 months, at least 3 months, at least 4 months, at least 5 months, at least 6 months, at least 9 months, at least 1 year, at least 2 years, at least 5 years, or longer. In other embodiments of any of the compositions and methods provided, the effective amount is one which produces a measurable desired tolerogenic immune response, for example, a measurable decrease in an immune response (e.g., to a rFVIIIrp), for at least 1 week, at least 2 weeks, at least 1 month, at least 2 months, at least 3 months, at least 4 months, at least 5 months, at least 6 months, at least 9 months, at least 1 year, at least 2 years, at least 5 years, or longer.

Effective amount will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner.

“Couple” or “Coupled” or “Couples” (and the like) means to chemically associate one entity (for example a moiety) with another. In some embodiments, the coupling is covalent, meaning that the coupling occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of coupling.

“Derived” means prepared from a material or use of information such as sequence related to a material but is not “obtained” from the material.

“Dosage form” means a pharmacologically and/or immunologically active material in a medium, carrier, vehicle, or device suitable for administration to a subject.

“Epitope”, also known as an antigenic determinant, is the part of an antigen that is recognized by the immune system, specifically by, for example, antibodies, B cells, or T cells.

As used herein, “MHC Class II-restricted epitopes” (or similar derivations) are epitopes that are presented to immune cells by MHC class II molecules found on antigen-presenting cells (APCs), for example, on professional antigen-presenting immune cells, such as on macrophages, B cells, and dendritic cells, or on non-hematopoietic cells, such as hepatocytes.

“Maintenance dose” refers to a dose that is administered to a subject, after an initial dose has resulted in the minimization or reduction of an undesired immune response in a subject, to sustain a desired tolerogenic response. A maintenance dose, for example, are one that maintains the tolerogenic effect achieved after the initial dose, prevents an undesired immune response in the subject, or prevents the subject becoming a subject at risk of experiencing an undesired immune response, including an undesired level of an immune response. In some embodiments, the maintenance dose is one that is sufficient to sustain an appropriate level of a desired immune response.

“Pharmaceutically acceptable excipient” means a pharmacologically inactive material used together with the recited peptides and carriers to formulate the inventive compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Protocol” refers to any dosing regimen of one or more substances to a subject. A dosing regimen may include the amount, frequency and/or mode of administration. In some embodiments, such a protocol may be used to administer one or more compositions of the invention to one or more subjects. Immune responses in these subjects can then be assessed to determine whether or not the protocol was effective in reducing an undesired immune response or generating a desired immune response (e.g., the promotion of a tolerogenic effect). Any other therapeutic and/or prophylactic effect may also be assessed instead of or in addition to the aforementioned immune responses. Whether or not a protocol had a desired effect are determined using any of the methods provided herein or otherwise known in the art. For example, a blood sample may be obtained from a subject to which a composition provided herein has been administered according to a specific protocol in order to determine whether or not specific inhibitors to FVIII were minimized, reduced, generated, or prevented. Useful methods for detecting the presence and/or number of inhibitors include ELISA assays, ELISPOT assays, and other similar type assays.

“Haplotype” refers to a combination of DNA sequences that are closely linked on one chromosome and are commonly inherited together. The gene encoding FVIII (F8) is polymorphic in the human population, yet there are four common non-synonymous single nucleotide polymorphisms (nsSNPs), that together with two infrequent nsSNPs define eight haplotypes of the F8 gene, referred to as haplotype (H)1, H2, H3, H4, H5, H6, H7, and H8. (Viel, K. R. et al. A sequence variation scan of the coagulation factor VIII (FVIII) structural gene and associations with plasma FVIII activity levels. Blood 109, 3713-3724 (2007); Howard, T. E. et al. Haemophilia management: time to get personal? Haemophilia 17, 721-728 (2011); Viel, K. R. et al. Inhibitors of factor VIII in black patients with hemophilia. N Engl J Med 360, 1618-1627 (2009))

“B-domain deleted FVIII” (BDD-FVIII or BDDFVIII) or the like refers to a protein that by virtue of recombinant genetic engineering comprises a FVIII protein in which the B domain of FVIII or some portion of the B domain of FVIII has been removed from the sequence of FVIII resulting in a functional recombinant FVIII protein. (Toole, J. J. et al. A large region (approximately equal to 95 kDa) of human factor VIII is dispensable for in vitro procoagulant activity. Proc Natl Acad Sci USA 83, 5939-5942 (1986)).

“Synthetic linker” refers to a sequence of DNA that by virtue of recombinant DNA techniques is introduced into the gene-encoding sequence of a gene, which DNA sequence is not present in the naturally-occurring sequence of the gene, and which DNA sequence serves the purpose of tying together an upstream and downstream portion of the gene and is necessitated when using recombinant DNA techniques to delete a domain or a portion of a domain of the gene.

“Single nucleotide polymorphism” (SNP) refers to a variation of one nucleotide (Adenine, Guanine, Cytosine, or Thymine) in the DNA sequence on a chromosome in the genome of an individual that differs from the nucleotide in the DNA sequence of either another chromosome of that individual or a chromosome of another individual.

“Non-synonymous single nucleotide polymorphism” (nsSNP or ns-SNP) refers to a SNP in the gene-encoding region of a chromosome that by the nature of its position in the gene-encoding region of a chromosome yields a change in the amino acid sequence of the protein encoded by the gene.

“Amino acid reference locus (AARL)” refers to a position within the FVIIIrp (the amino acid reference locus or AARL in the context of 1-2332 possible positions for wild type FVIII) that serves as a reference point or points for the preparation of a set or sets of tolerance inducing peptides or TIPS that may incorporate T-cell epitopes capable of inducing immune tolerance of, or the prevention, reduction, or elimination of anti FVIII inhibitor development by the subject to an FVIIIrp. An AARL occurs at a locus where there is a structural difference between the FVIIIrp and the sFVIII. The difference may arise due to haplotypic variance between the FVIIIrp and sFVIII, a mutation in the sFVIII, a private polymorphism in the sFVIII or another structural anomaly in the sFVIII. The first peptide in a TIP set where each peptide has length X, will be an amino acid residue which is identical to the AARL. In such as a TIP set, the second TIP will be derived so that the length of the TIP remains X, but the AARL locus is shifted one position upstream with reference to the FVIIIrp, the third TIP will be derived so that the length of the TIP remains X but the AARL locus is shifted two positions upstream of its original locus with reference to the FVIIIrp and so forth. TIP sets so derived will collectively overlap a contiguous portion of the rFVIIIrp sequence spanning a length of 2x−1 residues.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

The present invention may be understood more readily by reference to the following detailed description of embodiments of the invention and to the Figures and their previous and following description.

General

Blood clotting begins when platelets adhere to the cut wall of an injured blood vessel at a lesion site. Subsequently, in a cascade of enzymatically regulated reactions, soluble fibrinogen molecules are converted by the enzyme thrombin to insoluble strands of fibrin that hold the platelets together in a thrombus. At each step in the cascade, a protein precursor is converted to a protease that cleaves the next protein precursor in the series. Co-factors are required at most of the steps. FVIII circulates as an inactive precursor in blood, bound tightly and non-covalently to von Willebrand factor. FVIII is proteolytically activated by thrombin or factor Xa, which dissociates it from von Willebrand factor and activates its procoagulant function in the cascade. In its active form, the protein factor VIIIa is a cofactor that increases the catalytic efficiency of factor IXa toward factor X activation by several orders of magnitude.

People with deficiencies in FVIII or inhibitors against FVIII who are not treated with FVIII suffer uncontrolled internal bleeding that may cause a range of serious symptoms, from inflammatory reactions in joints to early death. Severe hemophiliacs, who number about 10,000 in the United States, can be treated with infusion of plasma derived (pd) or recombinant FVIII, which will restore the blood's normal clotting ability if administered with sufficient frequency and concentration. The classical definition of FVIII is that substance present in normal blood plasma that corrects the clotting defect in plasma derived from individuals with hemophilia A.

The development of FVIII inhibitors has been, next to HIV and hepatitis, the most serious complication of hemophilia therapy. Although the recent production of highly purified and genetically engineered FVIII products has decreased the risk of these infections, the development of inhibitors remains a major therapeutic challenge. Because affected patients, usually children, are rendered resistant to conventional replacement therapy, control of hemostasis becomes difficult, resulting in substantial morbidity. Inhibitors (alloantibodies) are IgG antibodies, mostly of the IgG4 subclass, that bind to replacement FVIII and interfere with its pro-coagulant function. Clinically, patients with inhibitors are classified into high and low responders according to the strength of the anamnestic response they experience when they are re-exposed to FVIII. The goals of therapy in these patients are to control severe acute bleeding and to eradicate the inhibitor.

Another strategy for coping with inhibitors is to attempt to induce immune tolerance (ITI) to a particular FVIIIrp. ITI involves frequent exposure to the FVIIIrp over extended periods of time and is not always successful. The large amounts of factor needed for successful ITI render it cost prohibitive in many circumstances.

Our current understanding suggests that an immunogenic CD4+ T-cell response to an exogenous protein requires that: (i) at least one of the peptides derived by proteolytic processing of the infused protein must be foreign (non-self) to the patient; (ii) at least one of the distinct isomers of class-II human-leukocyte antigens (HLA-II) comprising the subject's individual MHC-class-II (MHC-II) repertoire must be able to bind a foreign peptide with sufficient affinity and stability so that it can be presented by the antigen-presenting cells (APCs); (iii) at least one of the subject's subpopulations of CD4+ T cells has a T-cell antigen receptor (TCR) capable of functionally productive binding to an HLA-II/foreign-FVIII-peptide complex; and (iv) the above requirements occur in the presence of danger signals that induce expression of co-stimulatory molecules which provide a second signal to the T cells thereby driving the activation of the T cells.

By utilizing the same MHC class II peptides that induce an immune response, however, it is possible to induce long-term T-cell tolerance and mediate the activity of important immune cells such as regulatory T-cell, by inducing T-cell anergy and T-cell abortive activation in response to specific FVIIIrp epitopes. The present invention provides for the administration of tolerogenic peptides (termed tolerizing amino acids or TIPs) or sets of TIPs to a subject suffering from Hemophilia A in order to prevent, minimize, reduce, or eliminate the development of inhibitors in a subject who will receive, is receiving, or has received a recombinant FVIII replacement product, wherein TIPs are based on amino acid differences existing between the subject's endogenous FVIII protein and the recombinant FVIII replacement product. At least one TIP from a set of TIPs is administered, or alternatively the entire TIP set is administered, wherein each set of TIPs comprises overlapping peptides based on an amino acid difference between the amino acid sequence of the sFVIII and the FVIIIrp. In creating the set of TIPs of the present invention, a specific differing sFVIII amino acid is identified and the corresponding FVIIIrp positional equivalent wild-type amino acids (i.e., the “reference locus”) is used to create a set of between about 9 to 22 overlapping peptides, each containing a reference locus, for each particular reference locus identified, wherein each set of overlapping peptides collectively span a FVIIIrp amino acid sequence both upstream and downstream of the reference locus. Some embodiments provide for the administration of one or more of the overlapping TIPs, and in some embodiments the entire TIP set, from each TIP set in order to prevent or limit the development of, or minimize, reduce, or eliminate the existence of, inhibitors to the specific rFVIIIrp through the induction of a tolerogenic immune response.

Comparing sFVIII Amino Acid Sequence with rFVIIIrp Amino Acid Sequence

Current FVIII replacement therapies include the infusions of recombinant FVIII replacement products (rFVIIIrp) and, in some circumstance, plasma derived FVIII replacement products (pdFVIIIrp). rFVIIIrp is a biosynthetic blood coagulant prepared using recombinant DNA, and is structurally similar to endogenous wild-type human FVIII and produces the same biological effect. pdFVIIIrp is derived from pooled blood donations. Due to genetic variables within a subject including the individual's specific F8 mutation type, background FVIII haplotype, and HLA haplotype, however, the FVIIIrp mismatched amino acid may induce an immune response in the subject receiving the FVIIIrp, resulting in the development of inhibitors and the reduction in efficiency of the particular FVIIIrp. By determining the subject's endogenous FVIII protein amino acid sequence, and comparing it to the known amino acid sequence of FVIIIrp, for example a rFVIIIrp, the subject will receive, is receiving, or has received, amino acid differences between the sFVIII and FVIIIrp are identified, the corresponding locus of the particular amino acid difference in the sFVIII mapped (i.e., the reference locus), and sets of peptides based on the differences are created, wherein one or more peptides from each set, and in one embodiment the entire set, are administered in an effective amount to induce tolerance in the subject to at least one reference locus containing epitope.

FVIII is synthesized in the liver and the primary translation product of 2332 amino acids undergoes extensive post-translational modification, including N- and O-linked glycosylation, sulfation, and proteolytic cleavage. The latter event divides the initial multi-domain protein (A1-A2-B-A3-C1-C2) into a heavy chain (A1-A2-B) and a light chain (A3-C1-C2) and the protein is secreted as a two-chain molecule associated through a metal ion bridge (Lenting et al., The life cycle of coagulation FVIII in view of its structure and function. Blood 1998; 92: 3983-96).

Over 2100 unique mutations have been identified in the human F8 gene, with over 980 of them being missense mutations, i.e., a point mutation wherein a single nucleotide is changed, resulting in a codon that codes for a different amino acid than its wild-type counterpart (see HAMSTeRS Database: http://hadb.org.uk/WebPages/PublicFiles/MutationSummary.htm).

In one aspect of the present invention, differences between a sFVIII and a FVIIIrp are identified and a set of tolerogenic peptides as described herein are derived. In one embodiment, the FVIIIrp is a rFVIIIrp. rFVIIIrp amino acid sequences are well known in the art and are all based on variants of functional wild-type FVIII proteins. The wild-type FVIII protein is 2332 amino acids in length, preceded by a 19 amino acid signal sequence which is cleaved prior to secretion. The FVIII wild-type amino acid sequence (SEQ ID NO: 1) without the signal sequence is provided for in Table 1, and forms the basis for the positioning or mapping of the reference loci described herein.

TABLE 1
Human Factor VIII Wild-Type Amino Acid Sequence (SEQ ID NO: 1)

```
                10 20 30 40 50 60
ATRRYYLGAV ELSWDYMQSD LGELPVDARF PPRVPKSFPF NTSVVYKKTL FVEFTDHLFN
                70 80 90 100 110 120
IAKPRPPWMG LLGPTIQAEV YDTVVITLKN MASHPVSLHA VGVSYWKASE GAEYDDQTSQ
              130 140 150 160 170 180
REKEDDKVFP GGSHTYVWQV LKENGPMASD PLCLTYSYLS HVDLVKDLNS GLIGALLVCR
              190 200 210 220 230 240
EGSLAKEKTQ TLHKFILLFA VFDEGKSWHS ETKNSLMQDR DAASARAWPK MHTVNGYVNR
              250 260 270 280 290 300
SLPGLIGCHR KSVYWHVIGM GTTPEVHSIF LEGHTFLVRN HRQASLEISP ITFLTAQTLL
              310 320 330 340 350 360
MDLGQFLLFC HISSHQHDGM EAYVKVDSCP EEPQLRMKNN EEAEDYDDDL TDSEMDVVRF
              370 380 390 400 410 420
DDDNSPSFIQ IRSVAKKHPK TWVHYIAAEE EDWDYAPLVL APDDRSYKSQ YLNNGPQRIG
              430 440 450 460 470 480
RKYKKVRFMA YTDETFKTRE AIQHESGILG PLLYGEVGDT LLIIFKNQAS RPYNIYPHGI
              490 500 510 520 530 540
TDVRPLYSRR LPKGVKHLKD FPILPGEIFK YKWTVTVEDG PTKSDPRCLT RYYSSFVNME
              550 560 570 580 590 600
RDLASGLIGP LLICYKESVD QRGNQIMSDK RNVILFSVFD ENRSWYLTEN IQRFLPNPAG
              610 620 630 640 650 660
VQLEDPEFQA SNIMHSINGY VFDSLQLSVC LHEVAYWYIL SIGAQTDFLS VFFSGYTFKH
              670 680 690 700 710 720
KMVYEDTLTL FPFSGETVFM SMENPGLWIL GCHNSDFRNR GMTALLKVSS CDKNTGDYYE
              730 740 750 760 770 780
DSYEDISAYL LSKNNAIEPR SFSQNSRHPS TRQKQFNATT IPENDIEKTD PWFAHRTPMP
              790 800 810 820 830 840
KIQNVSSSDL LMLLRQSPTP HGLSLSDLQE AKYETFSDDP SPGAIDSNNS LSEMTHFRPQ
              850 860 870 880 890 900
LHHSGDMVFT PESGLQLRLN EKLGTTAATE LKKLDFKVSS TSNNLISTIP SDNLAAGTDN
              910 920 930 940 950 960
TSSLGPPSMP VHYDSQLDTT LFGKKSSPLT ESGGPLSLSE ENNDSKLLES GLMNSQESSW
              970 980 990 1000 1010 1020
GKNVSSTESG RLFKGKRAHG PALLTKDNAL FKVSISLLKT NKTSNNSATN RKTHIDGPSL
            1030 1040 1050 1060 1070 1080
LIENSPSVWQ NILESDTEFK KVTPLIHDRM LMDKNATALR LNHMSNKTTS SKNMEMVQQK
            1090 1100 1110 1120 1130 1140
KEGPIPPDAQ NPDMSFFKML FLPESARWIQ RTHGKNSLNS GQGPSPKQLV SLGPEKSVEG
            1150 1160 1170 1180 1190 1200
QNFLSEKNKV VVGKGEFTKD VGLKEMVFPS SRNLFLTNLD NLHENNTHNQ EKKIQEEIEK
            1210 1220 1230 1240 1250 1260
KETLIQENVV LPQIHTVTGT KNFMKNLFLL STRQNVEGSY DGAYAPVLQD FRSLNDSTNR
            1270 1280 1290 1300 1310 1320
TKKHTAHFSK KGEEENLEGL GNQTKQIVEK YACTTRISPN TSQQNFVTQR SKRALKQFRL
            1330 1340 1350 1360 1370 1380
PLEETELEKR IIVDDTSTQW SKNMKHLTPS TLTQIDYNEK EKGAITQSPL SDCLTRSHSI
            1390 1400 1410 1420 1430 1440
PQANRSPLPI AKVSSFPSIR PIYLTRVLFQ DNSSHLPAAS YRKKDSGVQE SSHFLQGAKK
            1450 1460 1470 1480 1490 1500
NNLSLAILTL EMTGDQREVG SLGTSATNSV TYKKVENTVL PKPDLPKTSG KVELLPKVHI
            1510 1520 1530 1540 1550 1560
YQKDLFPTET SNGSPGHLDL VEGSLLQGTE GAIKWNEANR PGKVPFLRVA TESSAKTPSK
            1570 1580 1590 1600 1610 1620
LLDPLAWDNH YGTQIPKEEW KSQEKSPEKT AFKKKDTILS LNACESNHAI AAINEGQNKP
            1630 1640 1650 1660 1670 1680
EIEVTWAKQG RTERLCSQNP PVLKRHQREI TRTTLQSDQE EIDYDDTISV EMKKEDFDIY
            1690 1700 1710 1720 1730 1740
DEDENQSPRS FQKKTRHYFI AAVERLWDYG MSSSPHVLRN RAQSGSVPQF KKVVFQEFTD
            1750 1760 1770 1780 1790 1800
GSFTQPLYRG ELNEHLGLLG PYIRAEVEDN IMVTFRNQAS RPYSFYSSLI SYEEDQRQGA
            1810 1820 1830 1840 1850 1860
EPRKNFVKPN ETKTYFWKVQ HHMAPTKDEF DCKAWAYFSD VDLEKDVHSG LIGPLLVCHT
            1870 1880 1890 1900 1910 1920
NTLNPAHGRQ VTVQEFALFF TIFDETKSWY FTENMERNCR APCNIQMEDP TFKENYRFHA
            1930 1940 1950 1960 1970 1980
INGYIMDTLP GLVMAQDQRI RWYLLSMGSN ENIHSIHFSG HVFTVRKKEE YKMALYNLYP
            1990 2000 2010 2020 2030 2040
GVFLTVEMLP SKAGIWRVEC LIGEHLHAGM STLFLVYSNK CQTPLGMASG HIRDFQITAS
            2050 2060 2070 2080 2090 2100
GQYGQWAPKL ARLHYSGSIN AWSTKEPFSW IKVDLLAPMI IHGIKTQGAR QKFSSLYISQ
            2110 2120 2130 2140 2150 2160
FIIMYSLDGK KWQTYRGNST GTLMVFFGNV DSSGIKHNIF NPPIIARYIR LHPTHYSIRS
            2170 2180 2190 2200 2210 2220
TLRMELMGCD LNSCSMPLGM ESKAISDAQI TASSYFTNMF ATWSPSKARL HLQGRSNAWR
            2230 2240 2250 2260 2270 2280
PQVNNPKEWL QVDFQKTMKV TGVTTQGVKS LLTSMYVKEF LISSSQDGHQ WTLFFQNGKV
            2290 2300 2310 2320 2330
KVFQGNQDSF TPVVNSLDPP LLTRYLRIHP QSWVHQIALR MEVLGCEAQD LY
```

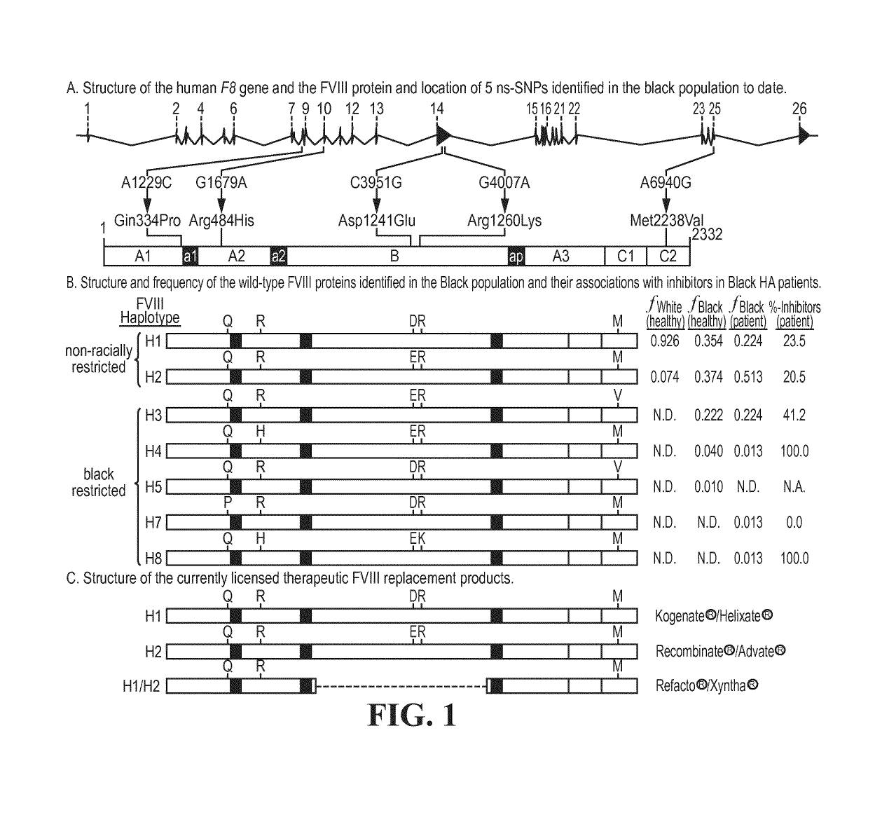

The human F8 gene is polymorphic and encodes several structurally distinct FVIII proteins referred to as haplotypes. Sequencing studies of the F8 gene have revealed four common nonsynonymous-single-nucleotide polymorphisms (nsSNPs) that, together with two infrequent ns-SNPs, encode eight distinct wild-type FVIII proteins referred to as haplotype H1, H2, H3, H4, H5, H6, H7, and H8. Seven of the variants—H1, H2, H3, H4, H5, H7, and H8—their associated nsSNP, their distribution in black and white populations, and inhibitor development are illustrated in FIG. 1.

The amino acid sequence of the H1 wild-type variant is provided for in Table 1. All currently available rFVIIIrp are based on either the H1 or H2 haplotype variant. Commercially available rFVIIIrp and their corresponding haplotype variant and corresponding ns-SNP location are provided for in FIG. 1, and include the H1 variants Kogenate® (Bayer) and Helixate® (ZLB Behring), the H2 variants Recombinate® (Baxter) and Advate® (Baxter), and the H1/H2 variant B-domain deleted Refacto® (Pfizer) and Xyntha® (Pfizer). The present invention, however, is not limited to the determination of reference loci contained in the commercially available products above, but can be applied to any FVIIIrp, including human/porcine hybrid rFVIIIrp, porcine rFVIIIrp, and alternative haplotype recombinant FVIII replacement products such as those identified in WO 2006/063031, which is incorporated by reference herein, and pdFVIIIrp. As previously described pdFVIIIrp are pooled from blood donors and consist of FVIII products primarily of the H1 haplotype.

Hemophilia A is caused by loss-of-function mutations in the F8 gene. The F8 gene is located on the X-chromosome and comprises 26 exons separated by 25 non-coding introns. Differences between a sFVIII and a FVIIIrp can result from, for example, missense mutations in the subject's F8 gene, nonsynonymous single-nucleotide polymorphisms (nsSNPs) (both well-known and “private” or individualized) or haplotypic variations between the sFVIII and FVIIIrp, inversions, for example intron 1 or 22 inversions, synthetic peptide inclusion due to B-domain deletions in the BDD-rFVIIIrp, and the like. Currently, over 2,100 unique mutations have been identified relating to HA.

Because the amino acid sequence of available rFVIIIrp are known, and differences in pdFVIIIrp are determined, differences (or mismatches) between the subject's endogenous FVIII protein sequence and FVIIIrp are readily identifiable using common techniques known in the art. The reference locus of the FVIIIrp (that is, the amino acid difference contained in the FVIIIrp) of the TIPs described herein can positionally correlate with an amino acid substitution in the sFVIII caused by a missense mutation in the subject's F8 gene. Identification of a subject's missense mutation are readily made by using techniques known in the art. For example, DNA from the subject are extracted from leukocytes in whole blood and all the endogenous coding regions and splice junctions of the factor VIII gene are analyzed by restriction analysis, direct DNA sequence analysis, Denaturing Gradient Gel Electrophoresis (DGGE), Chemical Mismatch Cleavage (CMC), and Denaturing High Performance Liquid Chromatography (DHPLC) (see, for example: Higuchi et al., Characterization of mutations in the factor VIII gene by direct sequencing of amplified genomic DNA. Genomics 1990: 6(1); 65-71, Schwaab et al. Mutations in hemophilia A. Br J Haematol 1993; 83: 450-458; Schwaab et al. Factor VIII gene mutations found by a comparative study of SSCP, DGGE, and CMC and their analysis on a molecular model of factor VIII protein. Hum Genet 1997; 101: 323-332; Oldenburg et al. Evaluation of DHPLC in the analysis of hemophilia A. J Biochem Biophys Methods 2001; 47: 39-51). Tables 2-87 identifies a number of known missense mutations, the resulting amino acid substitutions, and the corresponding rFVIIIrp reference loci (bolded and underlined). Additional missense mutations from which TIPs containing reference loci contemplated herein are directed to are identifiable through the HAMSTeRS database (Haemophilia A Mutation, Structure, Test and Resource Site) (http://hadb.org.uk/), which includes over 980 unique missense mutations. Tables 2-87 identify TIPs directed to a number of known missense mutations, wherein the reference locus of the rFVIIIrp correlating with each mis sense mutation is bolded and underlined.

Non-synonymous Single Nucleotide Polymorphism (nsSNP) differences between a sFVIII and a FVIIIrp can result in the development of inhibitors in certain subjects. For example, subjects with H3 or H4 background haplotypes (prevalent in the population of blacks of African descent) have a higher observable prevalence of inhibitor development than patients with H1 and H2 haplotypes, likely due to the fact that the only available rFVIIIrp products are of the H1 and H2 haplotype and the predominate haplotype in pdFVIIIrp the H1 haplotype. The reference locus of the TIPs described herein can positionally correlate with a nsSNP difference contained in the sFVIII. For example, the nsSNP variants of the commercially available rFVIIIrp are readily identified. For example, FIG. 1 describes the nsSNP variants for a number of commercially available rFVIIIrp. In one embodiment, the nsSNP difference is a result of a known nsSNP. In one embodiment, the nsSNP difference is a result of a rare or previously unknown nsSNP within the sFVIII. The identification of nsSNPs is well known in the art (see, for example: Viel at al. Inhibitors of Factor VIII in Black Patients with Hemophilia. N Engl J Med 2009; 360(16): 1618-1627; WO 2006/063031, both incorporated herein by reference). In one embodiment, the reference locus is a result of a nsSNP difference at amino acid 113 in the FVIIIrp. In one embodiment, the difference at amino acid 113 in the FVIIIrp is a glutamic acid. In one embodiment, the reference locus is a result of a nsSNP difference at amino acid 334 in the FVIIIrp. In one embodiment, the difference at amino acid 334 in the FVIIIrp is a glutamine. In one embodiment, the reference locus is a result of a nsSNP difference at amino acid 387 in the FVIIIrp. In one embodiment, the difference at amino acid 387 in the FVIIIrp is a alanine. In one embodiment, the reference locus is a result of a nsSNP difference at amino acid 484 in the FVIIIrp. In one embodiment, the difference at amino acid 484 in the FVIIIrp is an arginine. In one embodiment, the reference locus is a result of a nsSNP difference at amino acid 776 in the FVIIIrp. In one embodiment, the difference at amino acid 776 in the FVIIIrp is an arginine. In one embodiment, the reference locus is a result of a nsSNP difference at amino acid 1107 in the FVIIIrp. In one embodiment, the difference at amino acid 1107 in the FVIIIrp is an arginine. In one embodiment, the reference locus is a result of a nsSNP difference at amino acid 1241 in the FVIIIrp. In one embodiment, the difference at amino acid 1241 in the FVIIIrp is an aspartic acid. In one embodiment, the difference at amino acid 1241 is a glutamic acid. In one embodiment, the reference locus is a result of a nsSNP difference at amino acid 1260 in the FVIIIrp. In one embodiment, the difference at amino acid 1260 in the FVIIIrp is an arginine. In one embodiment, the reference locus is a result of a nsSNP difference at amino acid 1462 in the FVIIIrp. In one embodiment, the difference at amino acid 1462 in the FVIIIrp is a lysine. In one embodiment, the reference locus is a result of a nsSNP difference at amino acid 1668 in the FVIIIrp. In one embodiment, the difference at amino acid 1668 in the FVIIIrp is an isoleucine. In one embodiment, the reference locus is a result of a nsSNP difference at amino acid 2004 in the FVIIIrp. In one embodiment, the difference at amino acid 2004 in the FVIIIrp is a glutamic acid. In one embodiment, the reference locus is a result of a nsSNP difference at amino acid 2223 in the FVIIIrp. In one embodiment, the difference at amino acid 2223 in the FVIIIrp is a valine. In one embodiment, the reference locus is a result of a nsSNP difference at amino acid 2238 in the FVIIIrp. In one embodiment, the difference at amino acid 2238 in the FVIIIrp is a methionine. In one embodiment, the reference locus is a result of a nsSNP difference at amino acid 2292 in the FVIIIrp. In one embodiment, the difference at amino acid 2292 in the FVIIIrp is a proline. Tables 88-101 identifies a number of known nsSNPs and their corresponding amino acid substitutions in differing haplotypes Tables 88-101 also identifies TIPs directed to a number of known nsSNPs, wherein the reference locus correlating with each nsSNP is bolded and underlined.

Molecular genetic studies have shown that development of inhibitors to factor VIII replacement products occurs most frequently in patients with severe hemophilia due to major gene lesions including inversions. In one embodiment, the reference locus of the TIPs describe herein positionally correlates with a differing amino acid sequence within the sFVIII caused by an inversion of intron 1 or intron 22. In one embodiment, the inversion is an inversion of intron 1. In one embodiment, the inversion is an inversion of intron 22. The identification of inversions is well known in the art (see, for example, Viel at al. Inhibitors of Factor VIII in Black Patients with Hemophilia. N Engl J Med 2009; 360(16): 1618-1627).

The reference locus of a TIP can positionally correlate with a differing amino acid sequence within the sFVIII caused by an inversion of intron 22. Generally, subjects with intron 22 inversion express the entire FVIII intracellularly, albeit on two separate polypeptides. Importantly, another gene, F8B, is also generally expressed in both normal and HA subjects. The expression product of the F8B gene, FVIIIB, has sequence identity with a portion of the C1 domain and the entire C2 domain of FVIII. The presence of this FVIIIB polypeptide is important from a tolerance standpoint as it serves as a source for any T cells epitope or B cell epitopes needed to support processes that occur in the thymus (T cell clonal deletion) and spleen (B cell anergy) to achieve central tolerance. The expression product of F8I22I starts at residue 1 and ends at residue 2124. The polypeptide expressed by the F8B begins at residue 2125 and ends at residue 2332. Accordingly subjects having the F8I22I have the requisite FVIII material to yield one or more FVIII peptides ending at or before residue 2124, the last amino acid encoded by exon 22, or beginning at or after residue 2125, the first amino acid encoded by exon 23. Any potential T cell epitope within such a peptide would be expected to be recognized as a self-antigen and not be immunogenic in the subject. Peptides spanning the junction between residues 2124 and 2125, if proteolyzed from a rFVIIIrp and presented by MHC class II molecules, however, would be “foreign” and potentially immunogenic T cell epitopes in an F8I22I subject. Because of this, all subjects having F8I22I have similar reference loci across residues 2124Val and 2125Met with respect to all currently available FVIIIrp. Table 102 identifies TIPs directed to this FVIIIrp MV reference locus (bolded and underlined).

The reference locus of a TIP can positionally correlate with a differing amino acid sequence within the sFVIII caused by the removal of the B-domain from a BDD-rFVIIIrp. In certain BDD-rFVIIIrp, a deletion of 894 internal codons and splicing codons 762 and 1657 creates a FVIII product containing 1438 amino acids. The BDD-rFVIIIrp contains a synthetic junctional 14-peptide sequence SFS-QNPPVLKRHQR formed by covalent attachment of the three N-terminal most residues of the B-domain, S⁷⁴¹F⁷⁴²S⁷⁴³, to the 11 C-terminal-most residues Q¹⁶³⁸N¹⁶³⁹P¹⁶⁴⁰P¹⁶⁴¹V¹⁶⁴²L¹⁶⁴³K¹⁶⁴⁴R¹⁶⁴⁵H¹⁶⁴⁶Q¹⁶⁴⁷R¹⁶⁴⁸. This synthetic linker creates 11 unique peptides across a 15 amino acid sequence within the BDD-rFVIIIrp, which have potential immunogenicity. Table 103 identifies TIPs directed to this BDD-rFVIIIrp synthetic linker wherein the rFVIIIrp reference locus is bolded and underlined.

Creation of Tolerance Inducing Peptide Sets

The present invention includes the identification of TIP sets directed to at least one reference locus, and compositions and methods of use of such TIP sets. Once the subject's endogenous FVIII amino acid sequence and rFVIIIrp amino acid sequence are compared and specific reference loci identified, sets of TIPs encompassing at least one reference locus are identified. Each peptide within a set contains a reference locus. The peptides within a TIP set are identical to a contiguous portion of the FVIIIrp, and, in certain embodiments, similar to the sFVIII except generally for the reference locus.

In general, each peptide of a TIP set will overlap a contiguous portion of the FVIIIrp across 2X−1 amino acids, where X is the length of the peptides contained in the set. Furthermore, the contiguous FVIIIrp amino acid sequence overlapped by the peptides will include X−1 amino acid residues upstream and X−1 amino acid residues downstream from the reference locus position within the FVIIIrp, wherein X is the length of the peptides contained in the set.

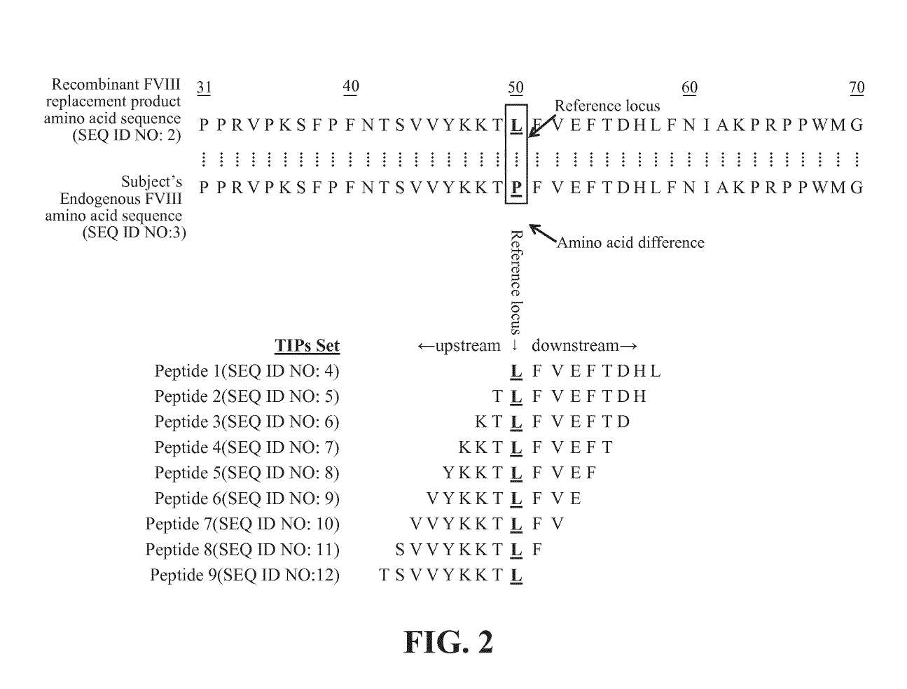

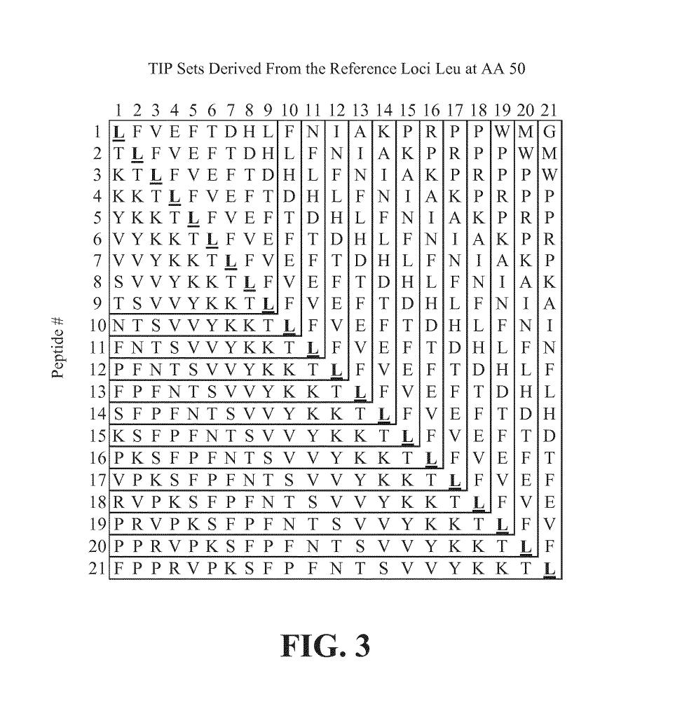

A further understanding of the identification of TIP sets contemplated herein may be gained by reference to, for illustrative purposes, FIGS. 2 and 3. For example, a subject may have a single missense mutation within their F8 gene resulting in a single amino acid substitution at a specific position within the endogenous FVIII protein that renders such protein defective. For example, the subject, due to a missense mutation, may have an amino acid substitution from Leu (the wild-type amino acid) to Pro (the missense substituted amino acid) at amino acid 50 within his endogenous FVIII protein. Comparatively, the FVIIIrp will not have that same substituted amino acid at this position, instead having the wild-type amino acid Leu at that position. Thus, comparing the sFVIII protein amino acid sequence (SEQ ID NO: 3) to the FVIIIrp (SEQ ID NO: 2) in this stance will identify Leu at amino acid 50 within the FVIIIrp as the reference locus.

Referring to FIG. 2, once the Leu at amino acid 50 is identified as reference locus, a set of 9 to 21 peptides ranging from 9 to 21 amino acids in length are identified, wherein each peptide in the set will contain the reference locus. Generally, the number of peptides identified in a TIP set is directly proportional to the selected peptide length. For example, if the TIP set is 9 amino acids in length, the set will contain 9 peptides, if the TIP set is 10 amino acids in length, the set will contain 10 peptides, and so forth. For illustrative purposes, a set of 9 peptides each of 9 amino acids in length are described in FIG. 2. Each peptide is identical to an amino acid portion of the FVIIIrp and, in the illustrative example, nearly identical to the homologous portion of the subject's endogenous FVIII protein, except at the reference locus. The first peptide of the set will contain the reference locus Leu in place of the subject's substituted amino acid Pro in its first position. In the example illustrated in FIG. 2, the first peptide in the set will have the sequence LFVEFTDHL (SEQ ID NO:4) and each successive peptide of the set will have the reference locus in a single upstream frame-shift position, so that that reference locus will be in position 2 of peptide 2 (TLFVRFTDH, SEQ ID NO:5), position 3 of peptide 3 (KTLFVEFTD, SEQ ID NO:6), and so, with the last peptide of the set having the reference locus in its last position (TSVVTKKTL, SEQ ID NO:12).

The peptides within a TIP set are identical to a contiguous portion of the FVIIIrp, and largely similar to the sFVIII, except generally for the reference locus. Each peptide will overlap a contiguous portion of the FVIIIrp across 2X−1 amino acids, where X is the length of the peptides contained in the set. For example, in the example illustrated in FIG. 2, each peptide illustrated is identical to a 9 amino acid portion of the FVIIIrp. Furthermore, the contiguous FVIIIrp amino acid sequence overlapped by the set of reference locus containing peptides is 2x−1 amino in length or 2(9)−(1)=17 amino acids. In addition, the contiguous FVIIIrp amino acid sequence overlapped will include X−1 amino acid residues upstream and X−1 amino acid residues downstream from the reference locus position within the FVIIIrp, wherein X is the length of the peptides contained in the set. In the example illustrated in FIG. 2, the amino acid sequence overlapped includes (9)−1=8 amino acids upstream of the reference locus Leu and (9)−(1) amino acids downstream of the reference locus Leu, so that the contiguous FVIIIrp amino acid sequence overlapped includes the 17 amino acid sequence TSVVYKKTLFVEFTDHL (SEQ ID NO: 13) corresponding to amino acids 42 to 58 of the FVIIIrp.

As previously described, the peptides identified in a TIP set are from about 9 amino acids in length to about 21 amino acids in length. The length of each peptide within each TIP set is generally the same, that is, all peptides within the TIP set will be the same amino acid length. In one embodiment, the length of the peptides within a particular TIP set is between about 9 amino acids and 21 amino acids. In one embodiment, the length of the peptides within a particular TIP set is at least 9 amino acids, 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids. In one embodiment, the length of the peptides within a particular TIP set is 9 amino acids. In one embodiment, the length of the peptides within a particular TIP set is 15 amino acids. In one embodiment, the length of the peptides within a particular TIP set is 17 amino acids. In one embodiment, the length of the peptides within a particular TIP set is 21 amino acids.

In some embodiments, the length of the peptides in the TIP set are sufficient to facilitate binding to a subject's class II human-leukocyte antigens comprising the subject's individual MHC-class II repertoire. The peptide length compares with that of naturally processed class II restricted epitopes (9 to 14 residues). Extra residues at either end of a CD4+ epitope sequence do not affect its attachment to the class II molecule binding cleft, which is open at both ends. Utilizing overlapping TIP sets of sizes greater than the MHC-II processing length, for example 15 amino acids, 16 amino acids, 17, amino acids, 18 amino acids, 19 amino acids, 20 amino acids, or 21 amino acids, reduces the risk of missing epitopes broken between peptides. In some embodiments, TIP sets of amino acids of length 15, 16, 17, 18, 19, 20, or 21 amino acids are contemplated herein.

For illustrative purposes, referring back to FIG. 2, the TIP set depicted is 9 peptides of 9 amino acids in length. As previously described, the TIP sets generally contemplated herein are from about 9 peptides of 9 amino acids in length to about 21 peptides of 21 amino acids in length. FIG. 3 is an illustrative example of a group of differing size TIP sets directed to the reference locus Leu at position 50 of the rFVIIIrp as depicted in FIG. 2. As illustrated in FIG. 3, using the reference locus, TIP sets of various peptide numbers and amino acid lengths are created through the frame-shifting process described previously. For example, FIG. 3 discloses a TIP set of 9 peptides of 9 amino acids in length. A TIP set are created comprising 10 peptides of 10 amino acids in length by using the frame-shifting process described above, resulting in an additional upstream and downstream amino acid residue from the rFVIIIrp being overlapped. The same process are used to create TIP sets of 11, 12, 13, 14, 15, 16, 17, 18, 19, 20, and 21 peptides of corresponding amino acid lengths.

The length of peptides between different TIP sets are the same length, or, in an alternative embodiment, different in length. For example, TIP sets for a subject with, for example, more than one amino acid differences between his FVIII protein and the FVIIIrp, are derived directed to each reference locus, wherein a first TIP set is directed to a first reference loci wherein the TIPs in the set are the same or a different amino acid length than the TIPs in a second TIP set directed to a second reference loci.

A TIP set can comprise one or more T cell epitopes. T cell epitopes are short antigenic peptides presented by major histocompatibility complex (MHC) receptors on the surfaces of antigen-presenting cells (APCs), such as dendritic cells, macrophages, and B cells. MHC surface receptors display both self-antigens and non-self (foreign) antigens, which are recognized by T cell receptors (TCRs) on the surfaces of T cells. Without being bound by a particular theory, it is believed that syngeneic apoptotic cells are phagocytosed by a population of tolerogenic DCs which present apoptotic cell-associated antigens in association with MHC II surface molecules under conditions that induce immunological tolerance to the antigen and suppress specific immunity. Methods of identifying T-cell epitopes for specific HLA phenotypes are generally known in the art: see, e.g., Nielsen et al. MHC class II epitope predictive algorithms. Immunology 2010; 130: 319-328; Wang et al. A systematic assessment of MHC class II peptide binding predictions and evaluation of a consensus approach. PLoS Comput Biol 2008; 4: e1000048; Mallios R R. Predicting class II MHC/peptide multi-level binding with an iterative stepwise discriminant analysis meta-algorithm. Bioinformatics 2001; 17: 942-948; Nielsen et al. Quantitative predictions of peptide binding to any HLA-DR molecule of known sequence: NetMHCIIpan. PLoS Comput Biol 2008; 4: e1000107.

In one aspect of the present invention, compositions comprising unique TIPs and TIP sets are provided for use in an immunogen tolerizing strategy. Compositions comprising a single TIP or set directed to a single reference locus, or multiple TIPs and TIP sets directed to one or more reference loci, are contemplated herein. In certain aspects, the TIPs and TIP sets described herein are associated with a carrier as described further below.

In one aspect of the present invention, compositions comprising one or more TIPs or TIP sets, wherein the reference loci of the TIP or TIP set is derived from a difference between a sFVIII and a rFVIIIrp as a result of missense mutations in the subject's F8 gene, nonsynonymous single-nucleotide polymorphisms (nsSNPs) or haplotypic variations between the sFVIII and rFVIIIrp, deletions, inversions, for example intron 1 or 22 inversions, administration of rFVIIIrp with synthetic linker sequences, for example BDD-rFVIIIrp, and the like, or combinations thereof, are contemplated herein. In one embodiment, the compositions comprise one or more TIPs or TIP sets, wherein the reference loci of the TIP or TIP set is derived from a difference between a sFVIII and a rFVIIIrp as a result of one or more missense mutations in the subject's F8 gene. In one embodiment, the compositions comprise one or more TIPs or TIP sets, wherein the reference loci of the TIP or TIP set is derived from a difference between a sFVIII and a rFVIIIrp as a result of one or more nonsynonymous single-nucleotide polymorphisms (nsSNPs) or haplotypic variations between the sFVIII and rFVIIIrp. In one embodiment, the compositions comprise one or more TIPs or TIP sets, wherein the reference loci of the TIP or TIP set is derived from a difference between a sFVIII and a rFVIIIrp as a result of one or more deletions within the subject's F8 gene. In one embodiment, the compositions comprise one or more TIPs or TIP sets, wherein the reference loci of the TIP or TIP set is derived from a difference between a sFVIII and a rFVIIIrp as a result of one or more inversions, for example intron 1 or 22 inversions. In one embodiment, the compositions comprise one or more TIPs or TIP sets, wherein the reference loci of the TIP or TIP set is derived from a difference between a sFVIII and a rFVIIIrp as a result of the use of rFVIIIrp with synthetic linker sequences, for example BDD-rFVIIIrp. In one embodiment, the compositions comprise one or more TIPs or TIP sets, wherein the reference loci of the TIP or TIP set is derived from a difference between a sFVIII and a rFVIIIrp as a result of a combination of any of the preceding.

In certain aspects of the present invention, compositions directed to specific TIPs and TIP sets described in Tables 2-87, and methods using the compositions thereof, are provided herein. In one embodiment, at least one or more TIPs comprising at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, or at least 21 amino acids, including at least one reference locus based on a sFVIII missense mutation, identified in Tables 2-87 are provided. In one embodiment, a TIP set comprising at least 9 peptides, at least 10 peptides, at least 11 peptides, at least 12 peptides, at least 13 peptides, at 14 peptides, 15 peptides, at least 16 peptides, at least 17 peptides, at least 18 peptides, at least 19 peptides, at least 20 peptides or at least 21 peptides, wherein the first peptide of the set comprises a reference locus at its first amino acid position, the second peptide of the set comprises a reference locus at its second amino acid position, and each successive peptide in the set comprises a reference locus at an amino acid position frame-shifted one position downstream from the reference locus position of the preceding peptide, and wherein the last peptide of the set has a reference locus in its last amino acid position, wherein the TIP sets are generated from the TIPs identified in Tables 2-87 (reference locus bolded and underlined), are provided herein. Tables 2-87 are provided below.

In particular embodiments, TIPs and TIP sets comprising reference locus based on missense mutations selected from the group consisting of Arg593Cys (Table 31), Tyr2105Cys (Table 67), Arg2150His (Table 69), Pro2300Leu (Table 84), Trp2229Cys (Table 79), Arg1997Pro (Table 57), or Asn2286Lys (Table 83) are provided herein. In certain aspects of the present invention, compositions directed to specific TIPs and TIP sets described in Tables 31, 57, 67, 69, 79, 83, or 84, and methods using the compositions thereof, are provided herein. In one embodiment, at least one or more TIPs comprising at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, or at least 21 amino acids, including at least one reference locus based on a sFVIII missense mutation, identified in Tables 31, 57, 67, 69, 79, 83, or 84 are provided. In one embodiment, a TIP set comprising at least 9 peptides, at least 10 peptides, at least 11 peptides, at least 12 peptides, at least 13 peptides, at 14 peptides, 15 peptides, at least 16 peptides, at least 17 peptides, at least 18 peptides, at least 19 peptides, at least 20 peptides or at least 21 peptides, wherein the first peptide of the set comprises a reference locus at its first amino acid position, the second peptide of the set comprises a reference locus at its second amino acid position, and each successive peptide in the set comprises a reference locus at an amino acid position frame-shifted one position downstream from the reference locus position of the preceding peptide, and wherein the last peptide of the set has a reference locus in its last amino acid position, wherein the TIP sets are generated from the TIPs identified in Tables 31, 57, 67, 69, 79, 83, or 84, are provided herein (reference locus bolded and underlined).

TABLE 2

| Reference | Missense | | | |
|---|---|---|---|---|
| locus position | nucleotide | FVIIIrp/sFVIII | | |
| within | change | amino acid | SEQ ID | |
| FVIIIrp | (wt/sFVIII) | difference | NO: | TIP Set |
| 50 | CTG/CCG | Leu/Pro | 14 | LFVEFTDHLFNIAKPRPPWMG |
| | | | 15 | TLFVEFTDHLFNIAKPRPPWM |
| | | | 16 | KTLFVEFTDHLFNIAKPRPPW |
| | | | 17 | KKTLFVEFTDHLFNIAKPRPP |
| | | | 18 | YKKTLFVEFTDHLFNIAKPRP |
| | | | 19 | VYKKTLFVEFTDHLFNIAKPR |
| | | | 20 | VVYKKTLFVEFTDHLFNIAKP |
| | | | 21 | SVVYKKTLFVEFTDHLFNIAK |
| | | | 22 | TSVVYKKTLFVEFTDHLFNIA |
| | | | 23 | NTSVVYKKTLFVEFTDHLFNI |
| | | | 24 | FNTSVVYKKTLFVEFTDHLFN |
| | | | 25 | PFNTSVVYKKTLFVEFTDHLF |
| | | | 26 | FPFNTSVVYKKTLFVEFTDHL |
| | | | 27 | SFPFNTSVVYKKTLFVEFTDH |
| | | | 28 | KSFPFNTSVVYKKTLFVEFTD |
| | | | 29 | PKSFPFNTSVVYKKTLFVEFT |
| | | | 30 | VPKSFPFNTSVVYKKTLFVEF |
| | | | 31 | RVPKSFPFNTSVVYKKTLFVE |
| | | | 32 | PRVPKSFPFNTSVVYKKTLFV |
| | | | 33 | PPRVPKSFPFNTSVVYKKTLF |
| | | | 34 | FPPRVPKSFPFNTSVVYKKTL |

TABLE 3

| Reference | Missense | | | |
|---|---|---|---|---|
| locus position | nucleotide | FVIIIrp/sFVIII | SEQ | |
| within | change | amino acid | ID | |
| FVIIIrp | (wt/sFVIII) | difference | NO: | TIP Set |
| 78 | GCT/CCT | Ala/Pro | 35 | AEVYDTVVITLKNMASHPVSL |
| | | | 36 | QAEVYDTVVITLKNMASHPVS |
| | | | 37 | IQAEVYDTVVITLKNMASHPV |
| | | | 38 | TIQAEVYDTVVITLKNMASHP |
| | | | 39 | PTIQAEVYDTVVITLKNMASH |
| | | | 40 | GPTIQAEVYDTVVITLKNMAS |
| | | | 41 | LGPTIQAEVYDTVVITLKNMA |
| | | | 42 | LLGPTIQAEVYDTVVITLKNM |
| | | | 43 | GLLGPTIQAEVYDTVVITLKN |
| | | | 44 | MGLLGPTIQAEVYDTVVITLK |
| | | | 45 | WMGLLGPTIQAEVYDTVVITL |
| | | | 46 | PWMGLLGPTIQAEVYDTVVIT |
| | | | 47 | PPWMGLLGPTIQAEVYDTVVI |
| | | | 48 | RPPWMGLLGPTIQAEVYDTVV |
| | | | 49 | PRPPWMGLLGPTIQAEVYDTV |
| | | | 50 | KPRPPWMGLLGPTIQAEVYDT |
| | | | 51 | AKPRPPWMGLLGPTIQAEVYD |
| | | | 52 | IAKPRPPWMGLLGPTIQAEVY |
| | | | 53 | NIAKPRPPWMGLLGPTIQAEV |
| | | | 54 | FNIAKPRPPWMGLLGPTIQAE |
| | | | 55 | LFNIAKPRPPWMGLLGPTIQA |

TABLE 4

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 102 | GGT/GCT | Gly/Ala | 56 | GVSYWKASEGAEYDDQTSQRE |
| | | | 57 | VGVSYWKASEGAEYDDQTSQR |
| | | | 58 | AVGVSYWKASEGAEYDDQTSQ |
| | | | 59 | HAVGVSYWKASEGAEYDDQTS |
| | | | 60 | LHAVGVSYWKASEGAEYDDQT |
| | | | 61 | SLHAVGVSYWKASEGAEYDDQ |
| | | | 62 | VSLHAVGVSYWKASEGAEYDD |
| | | | 63 | PVSLHAVGVSYWKASEGAEYD |
| | | | 64 | HPVSLHAVGVSYWKASEGAEY |
| | | | 65 | SHPVSLHAVGVSYWKASEGAE |
| | | | 66 | ASHPVSLHAVGVSYWKASEGA |
| | | | 67 | MASHPVSLHAVGVSYWKASEG |
| | | | 68 | NMASHPVSLHAVGVSYWKASE |
| | | | 69 | KNMASHPVSLHAVGVSYWKAS |
| | | | 70 | LKNMASHPVSLHAVGVSYWKA |
| | | | 71 | TLKNMASHPVSLHAVGVSYWK |
| | | | 72 | ITLKNMASHPVSLHAVGVSYW |
| | | | 73 | VITLKNMASHPVSLHAVGVSY |
| | | | 74 | VVITLKNMASHPVSLHAVGVS |
| | | | 75 | TVVITLKNMASHPVSLHAVGV |
| | | | 76 | DTVVITLKNMASHPVSLHAVG |

TABLE 5

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 113 | GAA/GAC | Glu/Asp | 77 | EYDDQTSQREKEDDKVFPGGS |
| | | | 78 | AEYDDQTSQREKEDDKVFPGG |
| | | | 79 | GAEYDDQTSQREKEDDKVFPG |
| | | | 80 | EGAEYDDQTSQREKEDDKVFP |
| | | | 81 | SEGAEYDDQTSQREKEDDKVF |
| | | | 82 | ASEGAEYDDQTSQREKEDDKV |
| | | | 83 | KASEGAEYDDQTSQREKEDDK |
| | | | 84 | WKASEGAEYDDQTSQREKEDD |
| | | | 85 | YWKASEGAEYDDQTSQREKED |
| | | | 86 | SYWKASEGAEYDDQTSQREKE |
| | | | 87 | VSYWKASEGAEYDDQTSQREK |
| | | | 88 | GVSYWKASEGAEYDDQTSQRE |
| | | | 89 | VGVSYWKASEGAEYDDQTSQR |
| | | | 90 | AVGVSYWKASEGAEYDDQTSQ |
| | | | 91 | HAVGVSYWKASEGAEYDDQTS |
| | | | 92 | LHAVGVSYWKASEGAEYDDQT |
| | | | 93 | SLHAVGVSYWKASEGAEYDDQ |
| | | | 94 | VSLHAVGVSYWKASEGAEYDD |
| | | | 95 | PVSLHAVGVSYWKASEGAEYD |
| | | | 96 | HPVSLHAVGVSYWKASEGAEY |
| | | | 97 | SHPVSLHAVGVSYWKASEGAE |

TABLE 6

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 154 | CTT/TTT | Leu/Phe | 98 | LTYSYLSHVDLVKDLNSGLIG |
| | | | 99 | CLTYSYLSHVDLVKDLNSGLI |
| | | | 100 | LCLTYSYLSHVDLVKDLNSGL |
| | | | 101 | PLCLTYSYLSHVDLVKDLNSG |
| | | | 102 | DPLCLTYSYLSHVDLVKDLNS |
| | | | 103 | SDPLCLTYSYLSHVDLVKDLN |
| | | | 104 | ASDPLCLTYSYLSHVDLVKDL |
| | | | 105 | MASDPLCLTYSYLSHVDLVKD |
| | | | 106 | PMASDPLCLTYSYLSHVDLVK |
| | | | 107 | GPMASDPLCLTYSYLSHVDLV |
| | | | 108 | NGPMASDPLCLTYSYLSHVDL |
| | | | 109 | ENGPMASDPLCLTYSYLSHVD |
| | | | 110 | KENGPMASDPLCLTYSYLSHV |
| | | | 111 | LKENGPMASDPLCLTYSYLSH |
| | | | 112 | VLKENGPMASDPLCLTYSYLS |
| | | | 113 | QVLKENGPMASDPLCLTYSYL |
| | | | 114 | WQVLKENGPMASDPLCLTYSY |
| | | | 115 | VWQVLKENGPMASDPLCLTYS |
| | | | 116 | YVWQVLKENGPMASDPLCLTY |
| | | | 117 | TYVWQVLKENGPMASDPLCLT |
| | | | 118 | HTYVWQVLKENGPMASDPLCL |

TABLE 7

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 163 | GAC/GTC | Asp/Val | 119 | DLVKDLNSGLIGALLVCREGS |
| | | | 120 | VDLVKDLNSGLIGALLVCREG |
| | | | 121 | HVDLVKDLNSGLIGALLVCRE |
| | | | 122 | SHVDLVKDLNSGLIGALLVCR |
| | | | 123 | LSHVDLVKDLNSGLIGALLVC |
| | | | 124 | YLSHVDLVKDLNSGLIGALLV |
| | | | 125 | SYLSHVDLVKDLNSGLIGALL |
| | | | 126 | YSYLSHVDLVKDLNSGLIGAL |
| | | | 127 | TYSYLSHVDLVKDLNSGLIGA |
| | | | 128 | LTYSYLSHVDLVKDLNSGLIG |
| | | | 129 | CLTYSYLSHVDLVKDLNSGLI |
| | | | 130 | LCLTYSYLSHVDLVKDLNSGL |
| | | | 131 | PLCLTYSYLSHVDLVKDLNSG |
| | | | 132 | DPLCLTYSYLSHVDLVKDLNS |
| | | | 133 | SDPLCLTYSYLSHVDLVKDLN |
| | | | 134 | ASDPLCLTYSYLSHVDLVKDL |
| | | | 135 | MASDPLCLTYSYLSHVDLVKD |
| | | | 136 | PMASDPLCLTYSYLSHVDLVK |
| | | | 137 | GPMASDPLCLTYSYLSHVDLV |
| | | | 138 | NGPMASDPLCLTYSYLSHVDL |
| | | | 139 | ENGPMASDPLCLTYSYLSHVD |

TABLE 8

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 198 | CTT/CAT | Leu/His | 140 | LFAVFDEGKSWHSETKNSLMQ |
| | | | 141 | LLFAVFDEGKSWHSETKNSLM |
| | | | 142 | ILLFAVFDEGKSWHSETKNSL |
| | | | 143 | FILLFAVFDEGKSWHSETKNS |
| | | | 144 | KFILLFAVFDEGKSWHSETKN |
| | | | 145 | HKFILLFAVFDEGKSWHSETK |
| | | | 146 | LHKFILLFAVFDEGKSWHSET |
| | | | 147 | TLHKFILLFAVFDEGKSWHSE |
| | | | 148 | QTLHKFILLFAVFDEGKSWHS |
| | | | 149 | TQTLHKFILLFAVFDEGKSWH |
| | | | 150 | KTQTLHKFILLFAVFDEGKSW |
| | | | 151 | EKTQTLHKFILLFAVFDEGKS |
| | | | 152 | KEKTQTLHKFILLFAVFDEGK |
| | | | 153 | AKEKTQTLHKFILLFAVFDEG |
| | | | 154 | LAKEKTQTLHKFILLFAVFDE |
| | | | 155 | SLAKEKTQTLHKFILLFAVFD |
| | | | 156 | GSLAKEKTQTLHKFILLFAVF |
| | | | 157 | EGSLAKEKTQTLHKFILLFAV |
| | | | 158 | REGSLAKEKTQTLHKFILLFA |
| | | | 159 | CREGSLAKEKTQTLHKFILLF |
| | | | 160 | VCREGSLAKEKTQTLHKFILL |

TABLE 9

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 204 | GAA/AAA | Glu/Lys | 161 | EGKSWHSETKNSLMQDRDAAS |
| | | | 162 | DEGKSWHSETKNSLMQDRDAA |
| | | | 163 | FDEGKSWHSETKNSLMQDRDA |
| | | | 164 | VFDEGKSWHSETKNSLMQDRD |
| | | | 165 | AVFDEGKSWHSETKNSLMQDR |
| | | | 166 | FAVFDEGKSWHSETKNSLMQD |
| | | | 167 | LFAVFDEGKSWHSETKNSLMQ |
| | | | 168 | LLFAVFDEGKSWHSETKNSLM |
| | | | 169 | ILLFAVFDEGKSWHSETKNSL |
| | | | 170 | FILLFAVFDEGKSWHSETKNS |
| | | | 171 | KFILLFAVFDEGKSWHSETKN |
| | | | 172 | HKFILLFAVFDEGKSWHSETK |
| | | | 173 | LHKFILLFAVFDEGKSWHSET |
| | | | 174 | TLHKFILLFAVFDEGKSWHSE |
| | | | 175 | QTLHKFILLFAVFDEGKSWHS |
| | | | 176 | TQTLHKFILLFAVFDEGKSWH |
| | | | 177 | KTQTLHKFILLFAVFDEGKSW |
| | | | 178 | EKTQTLHKFILLFAVFDEGKS |
| | | | 179 | KEKTQTLHKFILLFAVFDEGK |
| | | | 180 | AKEKTQTLHKFILLFAVFDEG |
| | | | 181 | LAKEKTQTLHKFILLFAVFDE |

TABLE 10

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 267 | CAC/CCC | His/Pro | 182 | HSIFLEGHTFLVRNHRQASLE |
| | | | 183 | VHSIFLEGHTFLVRNHRQASL |
| | | | 184 | EVHSIFLEGHTFLVRNHRQAS |
| | | | 185 | PEVHSIFLEGHTFLVRNHRQA |
| | | | 186 | TPEVHSIFLEGHTFLVRNHRQ |
| | | | 187 | TTPEVHSIFLEGHTFLVRNHR |
| | | | 188 | GTTPEVHSIFLEGHTFLVRNH |
| | | | 189 | MGTTPEVHSIFLEGHTFLVRN |
| | | | 190 | GMGTTPEVHSIFLEGHTFLVR |
| | | | 191 | IGMGTTPEVHSIFLEGHTFLV |
| | | | 192 | VIGMGTTPEVHSIFLEGHTFL |
| | | | 193 | HVIGMGTTPEVHSIFLEGHTF |
| | | | 194 | WHVIGMGTTPEVHSIFLEGHT |
| | | | 195 | YWHVIGMGTTPEVHSIFLEGH |
| | | | 196 | VYWHVIGMGTTPEVHSIFLEG |
| | | | 197 | SVYWHVIGMGTTPEVHSIFLE |
| | | | 198 | KSVYWHVIGMGTTPEVHSIFL |
| | | | 199 | RKSVYWHVIGMGTTPEVHSIF |
| | | | 200 | HRKSVYWHVIGMGTTPEVHSI |
| | | | 201 | CHRKSVYWHVIGMGTTPEVHS |
| | | | 202 | GCHRKSVYWHVIGMGTTPEVH |

TABLE 11

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 276 | TTT/CTT | Phe/Leu | 203 | FLVRNHRQASLEISPITFLTA |
| | | | 204 | TFLVRNHRQASLEISPITFLT |
| | | | 205 | HTFLVRNHRQASLEISPITFL |
| | | | 206 | GHTFLVRNHRQASLEISPITF |
| | | | 207 | EGHTFLVRNHRQASLEISPIT |
| | | | 208 | LEGHTFLVRNHRQASLEISPI |
| | | | 209 | FLEGHTFLVRNHRQASLEISP |
| | | | 210 | IFLEGHTFLVRNHRQASLEIS |
| | | | 211 | SIFLEGHTFLVRNHRQASLEI |
| | | | 212 | HSIFLEGHTFLVRNHRQASLE |
| | | | 213 | VHSIFLEGHTFLVRNHRQASL |
| | | | 214 | EVHSIFLEGHTFLVRNHRQAS |
| | | | 215 | PEVHSIFLEGHTFLVRNHRQA |
| | | | 216 | TPEVHSIFLEGHTFLVRNHRQ |
| | | | 217 | TTPEVHSIFLEGHTFLVRNHR |
| | | | 218 | GTTPEVHSIFLEGHTFLVRNH |
| | | | 219 | MGTTPEVHSIFLEGHTFLVRN |
| | | | 220 | GMGTTPEVHSIFLEGHTFLVR |
| | | | 221 | IGMGTTPEVHSIFLEGHTFLV |
| | | | 222 | VIGMGTTPEVHSIFLEGHTFL |
| | | | 223 | HVIGMGTTPEVHSIFLEGHTF |

TABLE 12

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 277 | CTT/TTT | Leu/Phe | 224 | LVRNHRQASLEISPITFLTAQ |
| | | | 225 | FLVRNHRQASLEISPITFLTA |
| | | | 226 | TFLVRNHRQASLEISPITFLT |
| | | | 227 | HTFLVRNHRQASLEISPITFL |
| | | | 228 | GHTFLVRNHRQASLEISPITF |
| | | | 229 | EGHTFLVRNHRQASLEISPIT |
| | | | 230 | LEGHTFLVRNHRQASLEISPI |
| | | | 231 | FLEGHTFLVRNHRQASLEISP |
| | | | 232 | IFLEGHTFLVRNHRQASLEIS |
| | | | 233 | SIFLEGHTFLVRNHRQASLEI |
| | | | 234 | HSIFLEGHTFLVRNHRQASLE |
| | | | 235 | VHSIFLEGHTFLVRNHRQASL |
| | | | 236 | EVHSIFLEGHTFLVRNHRQAS |
| | | | 237 | PEVHSIFLEGHTFLVRNHRQA |
| | | | 238 | TPEVHSIFLEGHTFLVRNHRQ |
| | | | 239 | TTPEVHSIFLEGHTFLVRNHR |
| | | | 240 | GTTPEVHSIFLEGHTFLVRNH |
| | | | 241 | MGTTPEVHSIFLEGHTFLVRN |
| | | | 242 | GMGTTPEVHSIFLEGHTFLVR |
| | | | 243 | IGMGTTPEVHSIFLEGHTFLV |
| | | | 244 | VIGMGTTPEVHSIFLEGHTFL |

TABLE 13

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 310 | TGT/TAT | Cys/Tyr | 245 | CHISSHQHDGMEAYVKVDSCP |
| | | | 246 | FCHISSHQHDGMEAYVKVDSC |
| | | | 247 | LFCHISSHQHDGMEAYVKVDS |
| | | | 248 | LLFCHISSHQHDGMEAYVKVD |
| | | | 249 | FLLFCHISSHQHDGMEAYVKV |
| | | | 250 | QFLLFCHISSHQHDGMEAYVK |
| | | | 251 | GQFLLFCHISSHQHDGMEAYV |
| | | | 252 | LGQFLLFCHISSHQHDGMEAY |
| | | | 253 | DLGQFLLFCHISSHQHDGMEA |
| | | | 254 | MDLGQFLLFCHISSHQHDGME |
| | | | 255 | LMDLGQFLLFCHISSHQHDGM |
| | | | 256 | LLMDLGQFLLFCHISSHQHDG |
| | | | 257 | TLLMDLGQFLLFCHISSHQHD |
| | | | 258 | QTLLMDLGQFLLFCHISSHQH |
| | | | 259 | AQTLLMDLGQFLLFCHISSHQ |
| | | | 260 | TAQTLLMDLGQFLLFCHISSH |
| | | | 261 | LTAQTLLMDLGQFLLFCHISS |
| | | | 262 | FLTAQTLLMDLGQFLLFCHIS |
| | | | 263 | TFLTAQTLLMDLGQFLLFCHI |
| | | | 264 | ITFLTAQTLLMDLGQFLLFCH |
| | | | 265 | PITFLTAQTLLMDLGQFLLFC |

TABLE 14

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 377 | AAG/ATG | Lys/Met | 266 | KHPKTWVHYIAAEEEDWDYAP |
| | | | 267 | KKHPKTWVHYIAAEEEDWDYA |
| | | | 268 | AKKHPKTWVHYIAAEEEDWDY |
| | | | 269 | VAKKHPKTWVHYIAAEEEDWD |
| | | | 270 | SVAKKHPKTWVHYIAAEEEDW |
| | | | 271 | RSVAKKHPKTWVHYIAAEEED |
| | | | 272 | IRSVAKKHPKTWVHYIAAEEE |
| | | | 273 | QIRSVAKKHPKTWVHYIAAEE |
| | | | 274 | IQIRSVAKKHPKTWVHYIAAE |
| | | | 275 | FIQIRSVAKKHPKTWVHYIAA |
| | | | 276 | SFIQIRSVAKKHPKTWVHYIA |
| | | | 277 | PSFIQIRSVAKKHPKTWVHYI |
| | | | 278 | SPSFIQIRSVAKKHPKTWVHY |
| | | | 279 | NSPSFIQIRSVAKKHPKTWVH |
| | | | 280 | DNSPSFIQIRSVAKKHPKTWV |
| | | | 281 | DDNSPSFIQIRSVAKKHPKTW |
| | | | 282 | DDDNSPSFIQIRSVAKKHPKT |
| | | | 283 | FDDDNSPSFIQIRSVAKKHPK |
| | | | 284 | RFDDDNSPSFIQIRSVAKKHP |
| | | | 285 | VRFDDDNSPSFIQIRSVAKKH |
| | | | 286 | VVRFDDDNSPSFIQIRSVAKK |

TABLE 15

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 384 | CAT/GAT | His/Asp | 287 | HYIAAEEEDWDYAPLVLAPDD |
| | | | 288 | VHYIAAEEEDWDYAPLVLAPD |
| | | | 289 | WVHYIAAEEEDWDYAPLVLAP |
| | | | 290 | TWVHYIAAEEEDWDYAPLVLA |
| | | | 291 | KTWVHYIAAEEEDWDYAPLVL |
| | | | 292 | PKTWVHYIAAEEEDWDYAPLV |
| | | | 293 | HPKTWVHYIAAEEEDWDYAPL |
| | | | 294 | KHPKTWVHYIAAEEEDWDYAP |
| | | | 295 | KKHPKTWVHYIAAEEEDWDYA |
| | | | 296 | AKKHPKTWVHYIAAEEEDWDY |
| | | | 297 | VAKKHPKTWVHYIAAEEEDWD |
| | | | 298 | SVAKKHPKTWVHYIAAEEEDW |
| | | | 299 | RSVAKKHPKTWVHYIAAEEED |
| | | | 300 | IRSVAKKHPKTWVHYIAAEEE |
| | | | 301 | QIRSVAKKHPKTWVHYIAAEE |
| | | | 302 | IQIRSVAKKHPKTWVHYIAAE |
| | | | 303 | FIQIRSVAKKHPKTWVHYIAA |
| | | | 304 | SFIQIRSVAKKHPKTWVHYIA |
| | | | 305 | PSFIQIRSVAKKHPKTWVHYI |
| | | | 306 | SPSFIQIRSVAKKHPKTWVHY |
| | | | 307 | NSPSFIQIRSVAKKHPKTWVH |

TABLE 16

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 393 | TGG/CGG | Trp/Arg | 308 | WDYAPLVLAPDDRSYKSQYLN |
| | | | 309 | DWDYAPLVLAPDDRSYKSQYL |
| | | | 310 | EDWDYAPLVLAPDDRSYKSQY |
| | | | 311 | EEDWDYAPLVLAPDDRSYKSQ |
| | | | 312 | EEEDWDYAPLVLAPDDRSYKS |
| | | | 313 | AEEEDWDYAPLVLAPDDRSYK |
| | | | 314 | AAEEEDWDYAPLVLAPDDRSY |
| | | | 315 | IAAEEEDWDYAPLVLAPDDRS |
| | | | 316 | YIAAEEEDWDYAPLVLAPDDR |
| | | | 317 | HYIAAEEEDWDYAPLVLAPDD |
| | | | 318 | VHYIAAEEEDWDYAPLVLAPD |
| | | | 319 | WVHYIAAEEEDWDYAPLVLAP |
| | | | 320 | TWVHYIAAEEEDWDYAPLVLA |
| | | | 321 | KTWVHYIAAEEEDWDYAPLVL |
| | | | 322 | PKTWVHYIAAEEEDWDYAPLV |
| | | | 323 | HPKTWVHYIAAEEEDWDYAPL |
| | | | 324 | KHPKTWVHYIAAEEEDWDYAP |
| | | | 325 | KKHPKTWVHYIAAEEEDWDYA |
| | | | 326 | AKKHPKTWVHYIAAEEEDWDY |
| | | | 327 | VAKKHPKTWVHYIAAEEEDWD |
| | | | 328 | SVAKKHPKTWVHYIAAEEEDW |

TABLE 17

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 396 | GCT/GTT | Ala/Val | 329 | APLVLAPDDRSYKSQYLNNGP |
| | | | 330 | YAPLVLAPDDRSYKSQYLNNG |
| | | | 331 | DYAPLVLAPDDRSYKSQYLNN |
| | | | 332 | WDYAPLVLAPDDRSYKSQYLN |
| | | | 333 | DWDYAPLVLAPDDRSYKSQYL |
| | | | 334 | EDWDYAPLVLAPDDRSYKSQY |
| | | | 335 | EEDWDYAPLVLAPDDRSYKSQ |
| | | | 336 | EEEDWDYAPLVLAPDDRSYKS |
| | | | 337 | AEEEDWDYAPLVLAPDDRSYK |
| | | | 338 | AAEEEDWDYAPLVLAPDDRSY |
| | | | 339 | IAAEEEDWDYAPLVLAPDDRS |
| | | | 340 | YIAAEEEDWDYAPLVLAPDDR |
| | | | 341 | HYIAAEEEDWDYAPLVLAPDD |
| | | | 342 | VHYIAAEEEDWDYAPLVLAPD |
| | | | 343 | WVHYIAAEEEDWDYAPLVLAP |
| | | | 344 | TWVHYIAAEEEDWDYAPLVLA |
| | | | 345 | KTWVHYIAAEEEDWDYAPLVL |
| | | | 346 | PKTWVHYIAAEEEDWDYAPLV |
| | | | 347 | HPKTWVHYIAAEEEDWDYAPL |
| | | | 348 | KHPKTWVHYIAAEEEDWDYAP |
| | | | 349 | KKHPKTWVHYIAAEEEDWDYA |

TABLE 18

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 405 | AGA/AGC | Arg/Ser | 350 | RSYKSQYLNNGPQRIGRKYKK |
| | | | 351 | DRSYKSQYLNNGPQRIGRKYK |
| | | | 352 | DDRSYKSQYLNNGPQRIGRKY |
| | | | 353 | PDDRSYKSQYLNNGPQRIGRK |
| | | | 354 | APDDRSYKSQYLNNGPQRIGR |
| | | | 355 | LAPDDRSYKSQYLNNGPQRIG |
| | | | 356 | VLAPDDRSYKSQYLNNGPQRI |
| | | | 357 | LVLAPDDRSYKSQYLNNGPQR |
| | | | 358 | PLVLAPDDRSYKSQYLNNGPQ |
| | | | 359 | APLVLAPDDRSYKSQYLNNGP |
| | | | 360 | YAPLVLAPDDRSYKSQYLNNG |
| | | | 361 | DYAPLVLAPDDRSYKSQYLNN |
| | | | 362 | WDYAPLVLAPDDRSYKSQYLN |
| | | | 363 | DWDYAPLVLAPDDRSYKSQYL |
| | | | 364 | EDWDYAPLVLAPDDRSYKSQY |
| | | | 365 | EEDWDYAPLVLAPDDRSYKSQ |
| | | | 366 | EEEDWDYAPLVLAPDDRSYKS |
| | | | 367 | AEEEDWDYAPLVLAPDDRSYK |
| | | | 368 | AAEEEDWDYAPLVLAPDDRSY |
| | | | 369 | IAAEEEDWDYAPLVLAPDDRS |
| | | | 370 | YIAAEEEDWDYAPLVLAPDDR |

TABLE 19

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 420 | GGT/GTT | Gly/Val | 371 | GRKYKKVRFMAYTDETFKTRE |
| | | | 372 | IGRKYKKVRFMAYTDETFKTR |
| | | | 373 | RIGRKYKKVRFMAYTDETFKT |
| | | | 374 | QRIGRKYKKVRFMAYTDETFK |
| | | | 375 | PQRIGRKYKKVRFMAYTDETF |
| | | | 376 | GPQRIGRKYKKVRFMAYTDET |
| | | | 377 | NGPQRIGRKYKKVRFMAYTDE |
| | | | 378 | NNGPQRIGRKYKKVRFMAYTD |
| | | | 379 | LNNGPQRIGRKYKKVRFMAYT |
| | | | 380 | YLNNGPQRIGRKYKKVRFMAY |
| | | | 381 | QYLNNGPQRIGRKYKKVRFMA |
| | | | 382 | SQYLNNGPQRIGRKYKKVRFM |
| | | | 383 | KSQYLNNGPQRIGRKYKKVRF |
| | | | 384 | YKSQYLNNGPQRIGRKYKKVR |
| | | | 385 | SYKSQYLNNGPQRIGRKYKKV |
| | | | 386 | RSYKSQYLNNGPQRIGRKYKK |
| | | | 387 | DRSYKSQYLNNGPQRIGRKYK |
| | | | 388 | DDRSYKSQYLNNGPQRIGRKY |
| | | | 389 | PDDRSYKSQYLNNGPQRIGRK |
| | | | 390 | APDDRSYKSQYLNNGPQRIGR |
| | | | 391 | LAPDDRSYKSQYLNNGPQRIG |

TABLE 20

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 439 | CGT/TGT | Arg/Cys | 392 | REAIQHESGILGPLLYGEVGD |
| | | | 393 | TREAIQHESGILGPLLYGEVG |
| | | | 394 | KTREAIQHESGILGPLLYGEV |
| | | | 395 | FKTREAIQHESGILGPLLYGE |
| | | | 396 | TFKTREAIQHESGILGPLLYG |
| | | | 397 | ETFKTREAIQHESGILGPLLY |
| | | | 398 | DETFKTREAIQHESGILGPLL |
| | | | 399 | TDETFKTREAIQHESGILGPL |
| | | | 400 | YTDETFKTREAIQHESGILGP |
| | | | 401 | AYTDETFKTREAIQHESGILG |
| | | | 402 | MAYTDETFKTREAIQHESGIL |
| | | | 403 | FMAYTDETFKTREAIQHESGI |
| | | | 404 | RFMAYTDETFKTREAIQHESG |
| | | | 405 | VRFMAYTDETFKTREAIQHES |
| | | | 406 | KVRFMAYTDETFKTREAIQHE |
| | | | 407 | KKVRFMAYTDETFKTREAIQH |
| | | | 408 | YKKVRFMAYTDETFKTREAIQ |
| | | | 409 | KYKKVRFMAYTDETFKTREAI |
| | | | 410 | RKYKKVRFMAYTDETFKTREA |
| | | | 411 | GRKYKKVRFMAYTDETFKTRE |
| | | | 412 | IGRKYKKVRFMAYTDETFKTR |

TABLE 21

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 451 | CCT/CGT | Pro/Arg | 413 | PLLYGEVGDTLLIIFKNQASR |
| | | | 414 | GPLLYGEVGDTLLIIFKNQAS |
| | | | 415 | LGPLLYGEVGDTLLIIFKNQA |
| | | | 416 | ILGPLLYGEVGDTLLIIFKNQ |
| | | | 417 | GILGPLLYGEVGDTLLIIFKN |
| | | | 418 | SGILGPLLYGEVGDTLLIIFK |
| | | | 419 | ESGILGPLLYGEVGDTLLIIF |
| | | | 420 | HESGILGPLLYGEVGDTLLII |
| | | | 421 | QHESGILGPLLYGEVGDTLLI |
| | | | 422 | IQHESGILGPLLYGEVGDTLL |
| | | | 423 | AIQHESGILGPLLYGEVGDTL |
| | | | 424 | EAIQHESGILGPLLYGEVGDT |
| | | | 425 | REAIQHESGILGPLLYGEVGD |
| | | | 426 | TREAIQHESGILGPLLYGEVG |
| | | | 427 | KTREAIQHESGILGPLLYGEV |
| | | | 428 | FKTREAIQHESGILGPLLYGE |
| | | | 429 | TFKTREAIQHESGILGPLLYG |
| | | | 430 | ETFKTREAIQHESGILGPLLY |
| | | | 431 | DETFKTREAIQHESGILGPLL |
| | | | 432 | TDETFKTREAIQHESGILGPL |
| | | | 433 | YTDETFKTREAIQHESGILGP |

TABLE 22

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 455 | GGG/GAG | Gly/Glu | 434 | GEVGDTLLIIFKNQASRPYNI |
| | | | 435 | YGEVGDTLLIIFKNQASRPYN |
| | | | 436 | LYGEVGDTLLIIFKNQASRPY |
| | | | 437 | LLYGEVGDTLLIIFKNQASRP |
| | | | 438 | PLLYGEVGDTLLIIFKNQASR |
| | | | 439 | GPLLYGEVGDTLLIIFKNQAS |
| | | | 440 | LGPLLYGEVGDTLLIIFKNQA |
| | | | 441 | ILGPLLYGEVGDTLLIIFKNQ |
| | | | 442 | GILGPLLYGEVGDTLLIIFKN |
| | | | 443 | SGILGPLLYGEVGDTLLIIFK |
| | | | 444 | ESGILGPLLYGEVGDTLLIIF |
| | | | 445 | HESGILGPLLYGEVGDTLLII |
| | | | 446 | QHESGILGPLLYGEVGDTLLI |
| | | | 447 | IQHESGILGPLLYGEVGDTLL |
| | | | 448 | AIQHESGILGPLLYGEVGDTL |
| | | | 449 | EAIQHESGILGPLLYGEVGDT |
| | | | 450 | REAIQHESGILGPLLYGEVGD |
| | | | 451 | TREAIQHESGILGPLLYGEVG |
| | | | 452 | KTREAIQHESGILGPLLYGEV |
| | | | 453 | FKTREAIQHESGILGPLLYGE |
| | | | 454 | TFKTREAIQHESGILGPLLYG |

TABLE 23

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 479 | GGA/AGA | Gly/Arg | 455 | GITDVRPLYSRRLPKGVKHLK |
| | | | 456 | HGITDVRPLYSRRLPKGVKHL |
| | | | 457 | PHGITDVRPLYSRRLPKGVKH |
| | | | 458 | YPHGITDVRPLYSRRLPKGVK |
| | | | 459 | IYPHGITDVRPLYSRRLPKGV |
| | | | 460 | NIYPHGITDVRPLYSRRLPKG |
| | | | 461 | YNIYPHGITDVRPLYSRRLPK |
| | | | 462 | PYNIYPHGITDVRPLYSRRLP |
| | | | 463 | RPYNIYPHGITDVRPLYSRRL |
| | | | 464 | SRPYNIYPHGITDVRPLYSRR |
| | | | 465 | ASRPYNIYPHGITDVRPLYSR |
| | | | 466 | QASRPYNIYPHGITDVRPLYS |
| | | | 467 | NQASRPYNIYPHGITDVRPLY |
| | | | 468 | KNQASRPYNIYPHGITDVRPL |
| | | | 469 | FKNQASRPYNIYPHGITDVRP |
| | | | 470 | IFKNQASRPYNIYPHGITDVR |
| | | | 471 | IIFKNQASRPYNIYPHGITDV |
| | | | 472 | LIIFKNQASRPYNIYPHGITD |
| | | | 473 | LLIIFKNQASRPYNIYPHGIT |
| | | | 474 | TLLIIFKNQASRPYNIYPHGI |
| | | | 475 | DTLLIIFKNQASRPYNIYPHG |

TABLE 24

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 494 | GGT/AGT | Gly/Ser | 476 | GVKHLKDFPILPGEIFKYKWT |
| | | | 477 | KGVKHLKDFPILPGEIFKYKW |
| | | | 478 | PKGVKHLKDFPILPGEIFKYK |
| | | | 479 | LPKGVKHLKDFPILPGEIFKY |
| | | | 480 | RLPKGVKHLKDFPILPGEIFK |
| | | | 481 | RRLPKGVKHLKDFPILPGEIF |
| | | | 482 | SRRLPKGVKHLKDFPILPGEI |
| | | | 483 | YSRRLPKGVKHLKDFPILPGE |
| | | | 484 | LYSRRLPKGVKHLKDFPILPG |
| | | | 485 | PLYSRRLPKGVKHLKDFPILP |
| | | | 486 | RPLYSRRLPKGVKHLKDFPIL |
| | | | 487 | VRPLYSRRLPKGVKHLKDFPI |
| | | | 488 | DVRPLYSRRLPKGVKHLKDFP |
| | | | 489 | TDVRPLYSRRLPKGVKHLKDF |
| | | | 490 | ITDVRPLYSRRLPKGVKHLKD |
| | | | 491 | GITDVRPLYSRRLPKGVKHLK |
| | | | 492 | HGITDVRPLYSRRLPKGVKHL |
| | | | 493 | PHGITDVRPLYSRRLPKGVKH |
| | | | 494 | YPHGITDVRPLYSRRLPKGVK |
| | | | 495 | IYPHGITDVRPLYSRRLPKGV |
| | | | 496 | NIYPHGITDVRPLYSRRLPKG |

TABLE 25

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 531 | CGC/TGC | Arg/Cys | 497 | RYYSSFVNMERDLASGLIGPL |
| | | | 498 | TRYYSSFVNMERDLASGLIGP |
| | | | 499 | LTRYYSSFVNMERDLASGLIG |
| | | | 500 | CLTRYYSSFVNMERDLASGLI |
| | | | 501 | RCLTRYYSSFVNMERDLASGL |
| | | | 502 | PRCLTRYYSSFVNMERDLASG |
| | | | 503 | DPRCLTRYYSSFVNMERDLAS |
| | | | 504 | SDPRCLTRYYSSFVNMERDLA |
| | | | 505 | KSDPRCLTRYYSSFVNMERDL |
| | | | 506 | TKSDPRCLTRYYSSFVNMERD |
| | | | 507 | PTKSDPRCLTRYYSSFVNMER |
| | | | 508 | GPTKSDPRCLTRYYSSFVNME |
| | | | 509 | DGPTKSDPRCLTRYYSSFVNM |
| | | | 510 | EDGPTKSDPRCLTRYYSSFVN |
| | | | 511 | VEDGPTKSDPRCLTRYYSSFV |
| | | | 512 | TVEDGPTKSDPRCLTRYYSSF |
| | | | 513 | VTVEDGPTKSDPRCLTRYYSS |
| | | | 514 | TVTVEDGPTKSDPRCLTRYYS |
| | | | 515 | WTVTVEDGPTKSDPRCLTRYY |
| | | | 516 | KWTVTVEDGPTKSDPRCLTRY |
| | | | 517 | YKWTVTVEDGPTKSDPRCLTR |

TABLE 26

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 531 | CGC/CAC | Arg/His | 518 | RYYSSFVNMERDLASGLIGPL |
| | | | 519 | TRYYSSFVNMERDLASGLIGP |
| | | | 520 | LTRYYSSFVNMERDLASGLIG |
| | | | 521 | CLTRYYSSFVNMERDLASGLI |
| | | | 522 | RCLTRYYSSFVNMERDLASGL |
| | | | 523 | PRCLTRYYSSFVNMERDLASG |
| | | | 524 | DPRCLTRYYSSFVNMERDLAS |
| | | | 525 | SDPRCLTRYYSSFVNMERDLA |
| | | | 526 | KSDPRCLTRYYSSFVNMERDL |
| | | | 527 | TKSDPRCLTRYYSSFVNMERD |
| | | | 528 | PTKSDPRCLTRYYSSFVNMER |
| | | | 529 | GPTKSDPRCLTRYYSSFVNME |
| | | | 530 | DGPTKSDPRCLTRYYSSFVNM |
| | | | 531 | EDGPTKSDPRCLTRYYSSFVN |
| | | | 532 | VEDGPTKSDPRCLTRYYSSFV |
| | | | 533 | TVEDGPTKSDPRCLTRYYSSF |
| | | | 534 | VTVEDGPTKSDPRCLTRYYSS |
| | | | 535 | TVTVEDGPTKSDPRCLTRYYS |
| | | | 536 | WTVTVEDGPTKSDPRCLTRYY |
| | | | 537 | KWTVTVEDGPTKSDPRCLTRY |
| | | | 538 | YKWTVTVEDGPTKSDPRCLTR |

TABLE 27

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 534 | TCT/CCT | Ser/Pro | 539 | SSFVNMERDLASGLIGPLLIC |
| | | | 540 | YSSFVNMERDLASGLIGPLLI |
| | | | 541 | YYSSFVNMERDLASGLIGPLL |
| | | | 542 | RYYSSFVNMERDLASGLIGPL |
| | | | 543 | TRYYSSFVNMERDLASGLIGP |
| | | | 544 | LTRYYSSFVNMERDLASGLIG |
| | | | 545 | CLTRYYSSFVNMERDLASGLI |
| | | | 546 | RCLTRYYSSFVNMERDLASGL |
| | | | 547 | PRCLTRYYSSFVNMERDLASG |
| | | | 548 | DPRCLTRYYSSFVNMERDLAS |
| | | | 549 | SDPRCLTRYYSSFVNMERDLA |
| | | | 550 | KSDPRCLTRYYSSFVNMERDL |
| | | | 551 | TKSDPRCLTRYYSSFVNMERD |
| | | | 552 | PTKSDPRCLTRYYSSFVNMER |
| | | | 553 | GPTKSDPRCLTRYYSSFVNME |
| | | | 554 | DGPTKSDPRCLTRYYSSFVNM |
| | | | 555 | EDGPTKSDPRCLTRYYSSFVN |
| | | | 556 | VEDGPTKSDPRCLTRYYSSFV |
| | | | 557 | TVEDGPTKSDPRCLTRYYSSF |
| | | | 558 | VTVEDGPTKSDPRCLTRYYSS |
| | | | 559 | TVTVEDGPTKSDPRCLTRYYS |

TABLE 28

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 534 | TCT/CCT | Ser/Pro | 560 | SSFVNMERDLASGLIGPLLIC |
| | | | 561 | YSSFVNMERDLASGLIGPLLI |
| | | | 562 | YYSSFVNMERDLASGLIGPLL |
| | | | 563 | RYYSSFVNMERDLASGLIGPL |
| | | | 564 | TRYYSSFVNMERDLASGLIGP |
| | | | 565 | LTRYYSSFVNMERDLASGLIG |
| | | | 566 | CLTRYYSSFVNMERDLASGLI |
| | | | 567 | RCLTRYYSSFVNMERDLASGL |
| | | | 568 | PRCLTRYYSSFVNMERDLASG |
| | | | 569 | DPRCLTRYYSSFVNMERDLAS |
| | | | 570 | SDPRCLTRYYSSFVNMERDLA |
| | | | 571 | KSDPRCLTRYYSSFVNMERDL |
| | | | 572 | TKSDPRCLTRYYSSFVNMERD |
| | | | 573 | PTKSDPRCLTRYYSSFVNMER |
| | | | 574 | GPTKSDPRCLTRYYSSFVNME |
| | | | 575 | DGPTKSDPRCLTRYYSSFVNM |
| | | | 576 | EDGPTKSDPRCLTRYYSSFVN |
| | | | 577 | VEDGPTKSDPRCLTRYYSSFV |
| | | | 578 | TVEDGPTKSDPRCLTRYYSSF |
| | | | 579 | VTVEDGPTKSDPRCLTRYYSS |
| | | | 580 | TVTVEDGPTKSDPRCLTRYYS |

TABLE 29

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 535 | AGT/GGT | Ser/Gly | 581 | SFVNMERDLASGLIGPLLICY |
| | | | 582 | SSFVNMERDLASGLIGPLLIC |
| | | | 583 | YSSFVNMERDLASGLIGPLLI |
| | | | 584 | YYSSFVNMERDLASGLIGPLL |
| | | | 585 | RYYSSFVNMERDLASGLIGPL |
| | | | 586 | TRYYSSFVNMERDLASGLIGP |
| | | | 587 | LTRYYSSFVNMERDLASGLIG |
| | | | 588 | CLTRYYSSFVNMERDLASGLI |
| | | | 589 | RCLTRYYSSFVNMERDLASGL |
| | | | 590 | PRCLTRYYSSFVNMERDLASG |
| | | | 591 | DPRCLTRYYSSFVNMERDLAS |
| | | | 592 | SDPRCLTRYYSSFVNMERDLA |
| | | | 593 | KSDPRCLTRYYSSFVNMERDL |
| | | | 594 | TKSDPRCLTRYYSSFVNMERD |
| | | | 595 | PTKSDPRCLTRYYSSFVNMER |
| | | | 596 | GPTKSDPRCLTRYYSSFVNME |
| | | | 597 | DGPTKSDPRCLTRYYSSFVNM |
| | | | 598 | EDGPTKSDPRCLTRYYSSFVN |
| | | | 599 | VEDGPTKSDPRCLTRYYSSFV |
| | | | 600 | TVEDGPTKSDPRCLTRYYSSF |
| | | | 601 | VTVEDGPTKSDPRCLTRYYSS |

TABLE 30

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 566 | ATA/ACA | Ile/Thr | 602 | IMSDKRNVILFSVFDENRSWY |
| | | | 603 | QIMSDKRNVILFSVFDENRSW |
| | | | 604 | NQIMSDKRNVILFSVFDENRS |
| | | | 605 | GNQIMSDKRNVILFSVFDENR |
| | | | 606 | RGNQIMSDKRNVILFSVFDEN |
| | | | 607 | QRGNQIMSDKRNVILFSVFDE |
| | | | 608 | DQRGNQIMSDKRNVILFSVFD |
| | | | 609 | VDQRGNQIMSDKRNVILFSVF |
| | | | 610 | SVDQRGNQIMSDKRNVILFSV |
| | | | 611 | ESVDQRGNQIMSDKRNVILFS |
| | | | 612 | KESVDQRGNQIMSDKRNVILF |
| | | | 613 | YKESVDQRGNQIMSDKRNVIL |
| | | | 614 | CYKESVDQRGNQIMSDKRNVI |
| | | | 615 | ICYKESVDQRGNQIMSDKRNV |
| | | | 616 | LICYKESVDQRGNQIMSDKRN |
| | | | 617 | LLICYKESVDQRGNQIMSDKR |
| | | | 618 | PLLICYKESVDQRGNQIMSDK |
| | | | 619 | GPLLICYKESVDQRGNQIMSD |
| | | | 620 | IGPLLICYKESVDQRGNQIMS |
| | | | 621 | LIGPLLICYKESVDQRGNQIM |
| | | | 622 | GLIGPLLICYKESVDQRGNQI |

TABLE 31

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 593 | CGC/TGC | Arg/Cys | 623 | RFLPNPAGVQLEDPEFQASNI |
| | | | 624 | QRFLPNPAGVQLEDPEFQASN |
| | | | 625 | IQRFLPNPAGVQLEDPEFQAS |
| | | | 626 | NIQRFLPNPAGVQLEDPEFQA |
| | | | 627 | ENIQRFLPNPAGVQLEDPEFQ |
| | | | 628 | TENIQRFLPNPAGVQLEDPEF |
| | | | 629 | LTENIQRFLPNPAGVQLEDPE |
| | | | 630 | YLTENIQRFLPNPAGVQLEDP |
| | | | 631 | WYLTENIQRFLPNPAGVQLED |
| | | | 632 | SWYLTENIQRFLPNPAGVQLE |
| | | | 633 | RSWYLTENIQRFLPNPAGVQL |
| | | | 634 | NRSWYLTENIQRFLPNPAGVQ |
| | | | 635 | ENRSWYLTENIQRFLPNPAGV |
| | | | 636 | DENRSWYLTENIQRFLPNPAG |
| | | | 637 | FDENRSWYLTENIQRFLPNPA |
| | | | 638 | VFDENRSWYLTENIQRFLPNP |
| | | | 639 | SVFDENRSWYLTENIQRFLPN |
| | | | 640 | FSVFDENRSWYLTENIQRFLP |
| | | | 641 | LFSVFDENRSWYLTENIQRFL |
| | | | 642 | ILFSVFDENRSWYLTENIQRF |
| | | | 643 | VILFSVFDENRSWYLTENIQR |

TABLE 32

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 612 | AAC/AGC | Asn/Ser | 644 | NIMHSINGYVFDSLQLSVCLH |
| | | | 645 | SNIMHSINGYVFDSLQLSVCL |
| | | | 646 | ASNIMHSINGYVFDSLQLSVC |
| | | | 647 | QASNIMHSINGYVFDSLQLSV |
| | | | 648 | FQASNIMHSINGYVFDSLQLS |
| | | | 649 | EFQASNIMHSINGYVFDSLQL |
| | | | 650 | PEFQASNIMHSINGYVFDSLQ |
| | | | 651 | DPEFQASNIMHSINGYVFDSL |
| | | | 652 | EDPEFQASNIMHSINGYVFDS |
| | | | 653 | LEDPEFQASNIMHSINGYVFD |
| | | | 654 | QLEDPEFQASNIMHSINGYVF |
| | | | 655 | VQLEDPEFQASNIMHSINGYV |
| | | | 656 | GVQLEDPEFQASNIMHSINGY |
| | | | 657 | AGVQLEDPEFQASNIMHSING |
| | | | 658 | PAGVQLEDPEFQASNIMHSIN |
| | | | 659 | NPAGVQLEDPEFQASNIMHSI |
| | | | 660 | PNPAGVQLEDPEFQASNIMHS |
| | | | 661 | LPNPAGVQLEDPEFQASNIMH |
| | | | 662 | FLPNPAGVQLEDPEFQASNIM |
| | | | 663 | RFLPNPAGVQLEDPEFQASNI |
| | | | 664 | QRFLPNPAGVQLEDPEFQASN |

TABLE 33

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 614 | ATG/ATT | Met/Ile | 665 | MHSINGYVFDSLQLSVCLHEV |
| | | | 666 | IMHSINGYVFDSLQLSVCLHE |
| | | | 667 | NIMHSINGYVFDSLQLSVCLH |
| | | | 668 | SNIMHSINGYVFDSLQLSVCL |
| | | | 669 | ASNIMHSINGYVFDSLQLSVC |
| | | | 670 | QASNIMHSINGYVFDSLQLSV |
| | | | 671 | FQASNIMHSINGYVFDSLQLS |
| | | | 672 | EFQASNIMHSINGYVFDSLQL |
| | | | 673 | PEFQASNIMHSINGYVFDSLQ |
| | | | 674 | DPEFQASNIMHSINGYVFDSL |
| | | | 675 | EDPEFQASNIMHSINGYVFDS |
| | | | 676 | LEDPEFQASNIMHSINGYVFD |
| | | | 677 | QLEDPEFQASNIMHSINGYVF |
| | | | 678 | VQLEDPEFQASNIMHSINGYV |
| | | | 679 | GVQLEDPEFQASNIMHSINGY |
| | | | 680 | AGVQLEDPEFQASNIMHSING |
| | | | 681 | PAGVQLEDPEFQASNIMHSIN |
| | | | 682 | NPAGVQLEDPEFQASNIMHSI |
| | | | 683 | PNPAGVQLEDPEFQASNIMHS |
| | | | 684 | LPNPAGVQLEDPEFQASNIMH |
| | | | 685 | FLPNPAGVQLEDPEFQASNIM |

TABLE 34

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 618 | AAT/AGT | Asn/Ser | 686 | NGYVFDSLQLSVCLHEVAYWY |
| | | | 687 | INGYVFDSLQLSVCLHEVAYW |
| | | | 688 | SINGYVFDSLQLSVCLHEVAY |
| | | | 689 | HSINGYVFDSLQLSVCLHEVA |
| | | | 690 | MHSINGYVFDSLQLSVCLHEV |
| | | | 691 | IMHSINGYVFDSLQLSVCLHE |
| | | | 692 | NIMHSINGYVFDSLQLSVCLH |
| | | | 693 | SNIMHSINGYVFDSLQLSVCL |
| | | | 694 | ASNIMHSINGYVFDSLQLSVC |
| | | | 695 | QASNIMHSINGYVFDSLQLSV |
| | | | 696 | FQASNIMHSINGYVFDSLQLS |
| | | | 697 | EFQASNIMHSINGYVFDSLQL |
| | | | 698 | PEFQASNIMHSINGYVFDSLQ |
| | | | 699 | DPEFQASNIMHSINGYVFDSL |
| | | | 700 | EDPEFQASNIMHSINGYVFDS |
| | | | 701 | LEDPEFQASNIMHSINGYVFD |
| | | | 702 | QLEDPEFQASNIMHSINGYVF |
| | | | 703 | VQLEDPEFQASNIMHSINGYV |
| | | | 704 | GVQLEDPEFQASNIMHSINGY |
| | | | 705 | AGVQLEDPEFQASNIMHSING |
| | | | 706 | PAGVQLEDPEFQASNIMHSIN |

TABLE 35

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 663 | GTC/TTC | Val/Phe | 707 | VYEDTLTLFPFSGETVFMSME |
| | | | 708 | MVYEDTLTLFPFSGETVFMSM |
| | | | 709 | KMVYEDTLTLFPFSGETVFMS |
| | | | 710 | HKMVYEDTLTLFPFSGETVFM |
| | | | 711 | KHKMVYEDTLTLFPFSGETVF |
| | | | 712 | FKHKMVYEDTLTLFPFSGETV |
| | | | 713 | TFKHKMVYEDTLTLFPFSGET |
| | | | 714 | YTFKHKMVYEDTLTLFPFSGE |
| | | | 715 | GYTFKHKMVYEDTLTLFPFSG |
| | | | 716 | SGYTFKHKMVYEDTLTLFPFS |
| | | | 717 | FSGYTFKHKMVYEDTLTLFPF |
| | | | 718 | FFSGYTFKHKMVYEDTLTLFP |
| | | | 719 | VFFSGYTFKHKMVYEDTLTLF |
| | | | 720 | SVFFSGYTFKHKMVYEDTLTL |
| | | | 721 | LSVFFSGYTFKHKMVYEDTLT |
| | | | 722 | FLSVFFSGYTFKHKMVYEDTL |
| | | | 723 | DFLSVFFSGYTFKHKMVYEDT |
| | | | 724 | TDFLSVFFSGYTFKHKMVYED |
| | | | 725 | QTDFLSVFFSGYTFKHKMVYE |
| | | | 726 | AQTDFLSVFFSGYTFKHKMVY |
| | | | 727 | GAQTDFLSVFFSGYTFKHKMV |

TABLE 36

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 684 | AAC/GAC | Asn/Asp | 728 | NPGLWILGCHNSDFRNRGMTA |
| | | | 729 | ENPGLWILGCHNSDFRNRGMT |
| | | | 730 | MENPGLWILGCHNSDFRNRGM |
| | | | 731 | SMENPGLWILGCHNSDFRNRG |
| | | | 732 | MSMENPGLWILGCHNSDFRNR |
| | | | 733 | FMSMENPGLWILGCHNSDFRN |
| | | | 734 | VFMSMENPGLWILGCHNSDFR |
| | | | 735 | TVFMSMENPGLWILGCHNSDF |
| | | | 736 | ETVFMSMENPGLWILGCHNSD |
| | | | 737 | GETVFMSMENPGLWILGCHNS |
| | | | 738 | SGETVFMSMENPGLWILGCHN |
| | | | 739 | FSGETVFMSMENPGLWILGCH |
| | | | 740 | PFSGETVFMSMENPGLWILGC |
| | | | 741 | FPFSGETVFMSMENPGLWILG |
| | | | 742 | LFPFSGETVFMSMENPGLWIL |
| | | | 743 | TLFPFSGETVFMSMENPGLWI |
| | | | 744 | LTLFPFSGETVFMSMENPGLW |
| | | | 745 | TLTLFPFSGETVFMSMENPGL |
| | | | 746 | DTLTLFPFSGETVFMSMENPG |
| | | | 747 | EDTLTLFPFSGETVFMSMENP |
| | | | 748 | YEDTLTLFPFSGETVFMSMEN |

TABLE 37

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 686 | GGT/CGT | Gly/Arg | 749 | GLWILGCHNSDFRNRGMTALL |
| | | | 750 | PGLWILGCHNSDFRNRGMTAL |
| | | | 751 | NPGLWILGCHNSDFRNRGMTA |
| | | | 752 | ENPGLWILGCHNSDFRNRGMT |
| | | | 753 | MENPGLWILGCHNSDFRNRGM |
| | | | 754 | SMENPGLWILGCHNSDFRNRG |
| | | | 755 | MSMENPGLWILGCHNSDFRNR |
| | | | 756 | FMSMENPGLWILGCHNSDFRN |
| | | | 757 | VFMSMENPGLWILGCHNSDFR |
| | | | 758 | TVFMSMENPGLWILGCHNSDF |
| | | | 759 | ETVFMSMENPGLWILGCHNSD |
| | | | 760 | GETVFMSMENPGLWILGCHNS |
| | | | 761 | SGETVFMSMENPGLWILGCHN |
| | | | 762 | FSGETVFMSMENPGLWILGCH |
| | | | 763 | PFSGETVFMSMENPGLWILGC |
| | | | 764 | FPFSGETVFMSMENPGLWILG |
| | | | 765 | LFPFSGETVFMSMENPGLWIL |
| | | | 766 | TLFPFSGETVFMSMENPGLWI |
| | | | 767 | LTLFPFSGETVFMSMENPGLW |
| | | | 768 | TLTLFPFSGETVFMSMENPGL |
| | | | 769 | DTLTLFPFSGETVFMSMENPG |

TABLE 38

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 701 | GGC/GAC | Gly/Asp | 770 | GMTALLKVSSCDKNTGDYYED |
| | | | 771 | RGMTALLKVSSCDKNTGDYYE |
| | | | 772 | NRGMTALLKVSSCDKNTGDYY |
| | | | 773 | RNRGMTALLKVSSCDKNTGDY |
| | | | 774 | FRNRGMTALLKVSSCDKNTGD |
| | | | 775 | DFRNRGMTALLKVSSCDKNTG |
| | | | 776 | SDFRNRGMTALLKVSSCDKNT |
| | | | 777 | NSDFRNRGMTALLKVSSCDKN |
| | | | 778 | HNSDFRNRGMTALLKVSSCDK |
| | | | 779 | CHNSDFRNRGMTALLKVSSCD |
| | | | 780 | GCHNSDFRNRGMTALLKVSSC |
| | | | 781 | LGCHNSDFRNRGMTALLKVSS |
| | | | 782 | ILGCHNSDFRNRGMTALLKVS |
| | | | 783 | WILGCHNSDFRNRGMTALLKV |
| | | | 784 | LWILGCHNSDFRNRGMTALLK |
| | | | 785 | GLWILGCHNSDFRNRGMTALL |
| | | | 786 | PGLWILGCHNSDFRNRGMTAL |
| | | | 787 | NPGLWILGCHNSDFRNRGMTA |
| | | | 788 | ENPGLWILGCHNSDFRNRGMT |
| | | | 789 | MENPGLWILGCHNSDFRNRGM |
| | | | 790 | SMENPGLWILGCHNSDFRNRG |

TABLE 39

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 708 | GTT/TTT | Val/Phe | 791 | VSSCDKNTGDYYEDSYEDISA |
| | | | 792 | KVSSCDKNTGDYYEDSYEDIS |
| | | | 793 | LKVSSCDKNTGDYYEDSYEDI |
| | | | 794 | LLKVSSCDKNTGDYYEDSYED |
| | | | 795 | ALLKVSSCDKNTGDYYEDSYE |
| | | | 796 | TALLKVSSCDKNTGDYYEDSY |
| | | | 797 | MTALLKVSSCDKNTGDYYEDS |
| | | | 798 | GMTALLKVSSCDKNTGDYYED |
| | | | 799 | RGMTALLKVSSCDKNTGDYYE |
| | | | 800 | NRGMTALLKVSSCDKNTGDYY |
| | | | 801 | RNRGMTALLKVSSCDKNTGDY |
| | | | 802 | FRNRGMTALLKVSSCDKNTGD |
| | | | 803 | DFRNRGMTALLKVSSCDKNTG |
| | | | 804 | SDFRNRGMTALLKVSSCDKNT |
| | | | 805 | NSDFRNRGMTALLKVSSCDKN |
| | | | 806 | HNSDFRNRGMTALLKVSSCDK |
| | | | 807 | CHNSDFRNRGMTALLKVSSCD |
| | | | 808 | GCHNSDFRNRGMTALLKVSSC |
| | | | 809 | LGCHNSDFRNRGMTALLKVSS |
| | | | 810 | ILGCHNSDFRNRGMTALLKVS |
| | | | 811 | WILGCHNSDFRNRGMTALLKV |

TABLE 40

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 731 | CTG/GTG | Leu/Val | 812 | LSKNNAIEPRSFSQNSRHPST |
| | | | 813 | LLSKNNAIEPRSFSQNSRHPS |
| | | | 814 | YLLSKNNAIEPRSFSQNSRHP |
| | | | 815 | AYLLSKNNAIEPRSFSQNSRH |
| | | | 816 | SAYLLSKNNAIEPRSFSQNSR |
| | | | 817 | ISAYLLSKNNAIEPRSFSQNS |
| | | | 818 | DISAYLLSKNNAIEPRSFSQN |
| | | | 819 | EDISAYLLSKNNAIEPRSFSQ |
| | | | 820 | YEDISAYLLSKNNAIEPRSFS |
| | | | 821 | SYEDISAYLLSKNNAIEPRSF |
| | | | 822 | DSYEDISAYLLSKNNAIEPRS |
| | | | 823 | EDSYEDISAYLLSKNNAIEPR |
| | | | 824 | YEDSYEDISAYLLSKNNAIEP |
| | | | 825 | YYEDSYEDISAYLLSKNNAIE |
| | | | 826 | DYYEDSYEDISAYLLSKNNAI |
| | | | 827 | GDYYEDSYEDISAYLLSKNNA |
| | | | 828 | TGDYYEDSYEDISAYLLSKNN |
| | | | 829 | NTGDYYEDSYEDISAYLLSKN |
| | | | 830 | KNTGDYYEDSYEDISAYLLSK |
| | | | 831 | DKNTGDYYEDSYEDISAYLLS |
| | | | 832 | CDKNTGDYYEDSYEDISAYLL |

TABLE 41

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1047 | CAT/TAT | His/Tyr | 833 | HDRMLMDKNATALRLNHMSNK |
| | | | 834 | IHDRMLMDKNATALRLNHMSN |
| | | | 835 | LIHDRMLMDKNATALRLNHMS |
| | | | 836 | PLIHDRMLMDKNATALRLNHM |
| | | | 837 | TPLIHDRMLMDKNATALRLNH |
| | | | 838 | VTPLIHDRMLMDKNATALRLN |
| | | | 839 | KVTPLIHDRMLMDKNATALRL |
| | | | 840 | KKVTPLIHDRMLMDKNATALR |
| | | | 841 | FKKVTPLIHDRMLMDKNATAL |
| | | | 842 | EFKKVTPLIHDRMLMDKNATA |
| | | | 843 | TEFKKVTPLIHDRMLMDKNAT |
| | | | 844 | DTEFKKVTPLIHDRMLMDKNA |
| | | | 845 | SDTEFKKVTPLIHDRMLMDKN |
| | | | 846 | ESDTEFKKVTPLIHDRMLMDK |
| | | | 847 | LESDTEFKKVTPLIHDRMLMD |
| | | | 848 | ILESDTEFKKVTPLIHDRMLM |
| | | | 849 | NILESDTEFKKVTPLIHDRML |
| | | | 850 | QNILESDTEFKKVTPLIHDRM |
| | | | 851 | WQNILESDTEFKKVTPLIHDR |
| | | | 852 | VWQNILESDTEFKKVTPLIHD |
| | | | 853 | SVWQNILESDTEFKKVTPLIH |

TABLE 42

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1732 | AAA/GAA | Lys/Glu | 854 | KVVFQEFTDGSFTQPLYRGEL |
| | | | 855 | KKVVFQEFTDGSFTQPLYRGE |
| | | | 856 | FKKVVFQEFTDGSFTQPLYRG |
| | | | 857 | QFKKVVFQEFTDGSFTQPLYR |
| | | | 858 | PQFKKVVFQEFTDGSFTQPLY |
| | | | 859 | VPQFKKVVFQEFTDGSFTQPL |
| | | | 860 | SVPQFKKVVFQEFTDGSFTQP |
| | | | 861 | GSVPQFKKVVFQEFTDGSFTQ |
| | | | 862 | SGSVPQFKKVVFQEFTDGSFT |
| | | | 863 | QSGSVPQFKKVVFQEFTDGSF |
| | | | 864 | AQSGSVPQFKKVVFQEFTDGS |
| | | | 865 | RAQSGSVPQFKKVVFQEFTDG |
| | | | 866 | NRAQSGSVPQFKKVVFQEFTD |
| | | | 867 | RNRAQSGSVPQFKKVVFQEFT |
| | | | 868 | LRNRAQSGSVPQFKKVVFQEF |
| | | | 869 | VLRNRAQSGSVPQFKKVVFQE |
| | | | 870 | HVLRNRAQSGSVPQFKKVVFQ |
| | | | 871 | PHVLRNRAQSGSVPQFKKVVF |
| | | | 872 | SPHVLRNRAQSGSVPQFKKVV |
| | | | 873 | SSPHVLRNRAQSGSVPQFKKV |
| | | | 874 | SSSPHVLRNRAQSGSVPQFKK |

TABLE 43

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1760 | GGG/GAG | Gly/Glu | 875 | GPYIRAEVEDNIMVTFRNQAS |
| | | | 876 | LGPYIRAEVEDNIMVTFRNQA |
| | | | 877 | LLGPYIRAEVEDNIMVTFRNQ |
| | | | 878 | GLLGPYIRAEVEDNIMVTFRN |
| | | | 879 | LGLLGPYIRAEVEDNIMVTFR |
| | | | 880 | HLGLLGPYIRAEVEDNIMVTF |
| | | | 881 | EHLGLLGPYIRAEVEDNIMVT |
| | | | 882 | NEHLGLLGPYIRAEVEDNIMV |
| | | | 883 | LNEHLGLLGPYIRAEVEDNIM |
| | | | 884 | ELNEHLGLLGPYIRAEVEDNI |
| | | | 885 | GELNEHLGLLGPYIRAEVEDN |
| | | | 886 | RGELNEHLGLLGPYIRAEVED |
| | | | 887 | YRGELNEHLGLLGPYIRAEVE |
| | | | 888 | LYRGELNEHLGLLGPYIRAEV |
| | | | 889 | PLYRGELNEHLGLLGPYIRAE |
| | | | 890 | QPLYRGELNEHLGLLGPYIRA |
| | | | 891 | TQPLYRGELNEHLGLLGPYIR |
| | | | 892 | FTQPLYRGELNEHLGLLGPYI |
| | | | 893 | SFTQPLYRGELNEHLGLLGPY |
| | | | 894 | GSFTQPLYRGELNEHLGLLGP |
| | | | 895 | DGSFTQPLYRGELNEHLGLLG |

TABLE 44

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1761 | CCA/CAA | Pro/Gln | 896 | PYIRAEVEDNIMVTFRNQASR |
| | | | 897 | GPYIRAEVEDNIMVTFRNQAS |
| | | | 898 | LGPYIRAEVEDNIMVTFRNQA |
| | | | 899 | LLGPYIRAEVEDNIMVTFRNQ |
| | | | 900 | GLLGPYIRAEVEDNIMVTFRN |
| | | | 901 | LGLLGPYIRAEVEDNIMVTFR |
| | | | 902 | HLGLLGPYIRAEVEDNIMVTF |
| | | | 903 | EHLGLLGPYIRAEVEDNIMVT |
| | | | 904 | NEHLGLLGPYIRAEVEDNIMV |
| | | | 905 | LNEHLGLLGPYIRAEVEDNIM |
| | | | 906 | ELNEHLGLLGPYIRAEVEDNI |
| | | | 907 | GELNEHLGLLGPYIRAEVEDN |
| | | | 908 | RGELNEHLGLLGPYIRAEVED |
| | | | 909 | YRGELNEHLGLLGPYIRAEVE |
| | | | 910 | LYRGELNEHLGLLGPYIRAEV |
| | | | 911 | PLYRGELNEHLGLLGPYIRAE |
| | | | 912 | QPLYRGELNEHLGLLGPYIRA |
| | | | 913 | TQPLYRGELNEHLGLLGPYIR |
| | | | 914 | FTQPLYRGELNEHLGLLGPYI |
| | | | 915 | SFTQPLYRGELNEHLGLLGPY |
| | | | 916 | GSFTQPLYRGELNEHLGLLGP |

TABLE 45

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1779 | GCC/CCC | Ala/Pro | 917 | ASRPYSFYSSLISYEEDQRQG |
| | | | 918 | QASRPYSFYSSLISYEEDQRQ |
| | | | 919 | NQASRPYSFYSSLISYEEDQR |
| | | | 920 | RNQASRPYSFYSSLISYEEDQ |
| | | | 921 | FRNQASRPYSFYSSLISYEED |
| | | | 922 | TFRNQASRPYSFYSSLISYEE |
| | | | 923 | VTFRNQASRPYSFYSSLISYE |
| | | | 924 | MVTFRNQASRPYSFYSSLISY |
| | | | 925 | IMVTFRNQASRPYSFYSSLIS |
| | | | 926 | NIMVTFRNQASRPYSFYSSLI |
| | | | 927 | DNIMVTFRNQASRPYSFYSSL |
| | | | 928 | EDNIMVTFRNQASRPYSFYSS |
| | | | 929 | VEDNIMVTFRNQASRPYSFYS |
| | | | 930 | EVEDNIMVTFRNQASRPYSFY |
| | | | 931 | AEVEDNIMVTFRNQASRPYSF |
| | | | 932 | RAEVEDNIMVTFRNQASRPYS |
| | | | 933 | IRAEVEDNIMVTFRNQASRPY |
| | | | 934 | YIRAEVEDNIMVTFRNQASRP |
| | | | 935 | PYIRAEVEDNIMVTFRNQASR |
| | | | 936 | GPYIRAEVEDNIMVTFRNQAS |
| | | | 937 | LGPYIRAEVEDNIMVTFRNQA |

TABLE 46

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1781 | CGT/CAT | Arg/His | 938 | RPYSFYSSLISYEEDQRQGAE |
| | | | 939 | SRPYSFYSSLISYEEDQRQGA |
| | | | 940 | ASRPYSFYSSLISYEEDQRQG |
| | | | 941 | QASRPYSFYSSLISYEEDQRQ |
| | | | 942 | NQASRPYSFYSSLISYEEDQR |
| | | | 943 | RNQASRPYSFYSSLISYEEDQ |
| | | | 944 | FRNQASRPYSFYSSLISYEED |
| | | | 945 | TFRNQASRPYSFYSSLISYEE |
| | | | 946 | VTFRNQASRPYSFYSSLISYE |
| | | | 947 | MVTFRNQASRPYSFYSSLISY |
| | | | 948 | IMVTFRNQASRPYSFYSSLIS |
| | | | 949 | NIMVTFRNQASRPYSFYSSLI |
| | | | 950 | DNIMVTFRNQASRPYSFYSSL |
| | | | 951 | EDNIMVTFRNQASRPYSFYSS |
| | | | 952 | VEDNIMVTFRNQASRPYSFYS |
| | | | 953 | EVEDNIMVTFRNQASRPYSFY |
| | | | 954 | AEVEDNIMVTFRNQASRPYSF |
| | | | 955 | RAEVEDNIMVTFRNQASRPYS |
| | | | 956 | IRAEVEDNIMVTFRNQASRPY |
| | | | 957 | YIRAEVEDNIMVTFRNQASRP |
| | | | 958 | PYIRAEVEDNIMVTFRNQASR |

TABLE 47

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1786 | TAT/TCT | Tyr/Ser | 959 | YSSLISYEEDQRQGAEPRKNF |
| | | | 960 | FYSSLISYEEDQRQGAEPRKN |
| | | | 961 | SFYSSLISYEEDQRQGAEPRK |
| | | | 962 | YSFYSSLISYEEDQRQGAEPR |
| | | | 963 | PYSFYSSLISYEEDQRQGAEP |
| | | | 964 | RPYSFYSSLISYEEDQRQGAE |
| | | | 965 | SRPYSFYSSLISYEEDQRQGA |
| | | | 966 | ASRPYSFYSSLISYEEDQRQG |
| | | | 967 | QASRPYSFYSSLISYEEDQRQ |
| | | | 968 | NQASRPYSFYSSLISYEEDQR |
| | | | 969 | RNQASRPYSFYSSLISYEEDQ |
| | | | 970 | FRNQASRPYSFYSSLISYEED |
| | | | 971 | TFRNQASRPYSFYSSLISYEE |
| | | | 972 | VTFRNQASRPYSFYSSLISYE |
| | | | 973 | MVTFRNQASRPYSFYSSLISY |
| | | | 974 | IMVTFRNQASRPYSFYSSLIS |
| | | | 975 | NIMVTFRNQASRPYSFYSSLI |
| | | | 976 | DNIMVTFRNQASRPYSFYSSL |
| | | | 977 | EDNIMVTFRNQASRPYSFYSS |
| | | | 978 | VEDNIMVTFRNQASRPYSFYS |
| | | | 979 | EVEDNIMVTFRNQASRPYSFY |

TABLE 48

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1828 | GAT/GGT | Asp/Gly | 980 | DEFDCKAWAYFSDVDLEKDVH |
| | | | 981 | KDEFDCKAWAYFSDVDLEKDV |
| | | | 982 | TKDEFDCKAWAYFSDVDLEKD |
| | | | 983 | PTKDEFDCKAWAYFSDVDLEK |
| | | | 984 | APTKDEFDCKAWAYFSDVDLE |
| | | | 985 | MAPTKDEFDCKAWAYFSDVDL |
| | | | 986 | HMAPTKDEFDCKAWAYFSDVD |
| | | | 987 | HHMAPTKDEFDCKAWAYFSDV |
| | | | 988 | QHHMAPTKDEFDCKAWAYFSD |
| | | | 989 | VQHHMAPTKDEFDCKAWAYFS |
| | | | 990 | KVQHHMAPTKDEFDCKAWAYF |
| | | | 991 | WKVQHHMAPTKDEFDCKAWAY |
| | | | 992 | FWKVQHHMAPTKDEFDCKAWA |
| | | | 993 | YFWKVQHHMAPTKDEFDCKAW |
| | | | 994 | TYFWKVQHHMAPTKDEFDCKA |
| | | | 995 | KTYFWKVQHHMAPTKDEFDCK |
| | | | 996 | TKTYFWKVQHHMAPTKDEFDC |
| | | | 997 | ETKTYFWKVQHHMAPTKDEFD |
| | | | 998 | NETKTYFWKVQHHMAPTKDEF |
| | | | 999 | PNETKTYFWKVQHHMAPTKDE |
| | | | 1000 | KPNETKTYFWKVQHHMAPTKD |

TABLE 49

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1854 | CCC/CTC | Pro/Leu | 1001 | PLLVCHTNTLNPAHGRQVTVQ |
| | | | 1002 | GPLLVCHTNTLNPAHGRQVTV |
| | | | 1003 | IGPLLVCHTNTLNPAHGRQVT |
| | | | 1004 | LIGPLLVCHTNTLNPAHGRQV |
| | | | 1005 | GLIGPLLVCHTNTLNPAHGRQ |
| | | | 1006 | SGLIGPLLVCHTNTLNPAHGR |
| | | | 1007 | HSGLIGPLLVCHTNTLNPAHG |
| | | | 1008 | VHSGLIGPLLVCHTNTLNPAH |
| | | | 1009 | DVHSGLIGPLLVCHTNTLNPA |
| | | | 1010 | KDVHSGLIGPLLVCHTNTLNP |
| | | | 1011 | EKDVHSGLIGPLLVCHTNTLN |
| | | | 1012 | LEKDVHSGLIGPLLVCHTNTL |
| | | | 1013 | DLEKDVHSGLIGPLLVCHTNT |
| | | | 1014 | VDLEKDVHSGLIGPLLVCHTN |
| | | | 1015 | DVDLEKDVHSGLIGPLLVCHT |
| | | | 1016 | SDVDLEKDVHSGLIGPLLVCH |
| | | | 1017 | FSDVDLEKDVHSGLIGPLLVC |
| | | | 1018 | YFSDVDLEKDVHSGLIGPLLV |
| | | | 1019 | AYFSDVDLEKDVHSGLIGPLL |
| | | | 1020 | WAYFSDVDLEKDVHSGLIGPL |
| | | | 1021 | AWAYFSDVDLEKDVHSGLIGP |

TABLE 50

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1890 | TAC/TGC | Tyr/Cys | 1022 | YFTENMERNCRAPCNIQMEDP |
| | | | 1023 | WYFTENMERNCRAPCNIQMED |
| | | | 1024 | SWYFTENMERNCRAPCNIQME |
| | | | 1025 | KSWYFTENMERNCRAPCNIQM |
| | | | 1026 | TKSWYFTENMERNCRAPCNIQ |
| | | | 1027 | ETKSWYFTENMERNCRAPCNI |
| | | | 1028 | DETKSWYFTENMERNCRAPCN |
| | | | 1029 | FDETKSWYFTENMERNCRAPC |
| | | | 1030 | IFDETKSWYFTENMERNCRAP |
| | | | 1031 | TIFDETKSWYFTENMERNCRA |
| | | | 1032 | FTIFDETKSWYFTENMERNCR |
| | | | 1033 | FFTIFDETKSWYFTENMERNC |
| | | | 1034 | LFFTIFDETKSWYFTENMERN |
| | | | 1035 | ALFFTIFDETKSWYFTENMER |
| | | | 1036 | FALFFTIFDETKSWYFTENME |
| | | | 1037 | EFALFFTIFDETKSWYFTENM |
| | | | 1038 | QEFALFFTIFDETKSWYFTEN |
| | | | 1039 | VQEFALFFTIFDETKSWYFTE |
| | | | 1040 | TVQEFALFFTIFDETKSWYFT |
| | | | 1041 | VTVQEFALFFTIFDETKSWYF |
| | | | 1042 | QVTVQEFALFFTIFDETKSWY |

TABLE 51

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1920 | GCA/GAA | Ala/Glu | 1043 | AINGYIMDTLPGLVMAQDQRI |
| | | | 1044 | HAINGYIMDTLPGLVMAQDQR |
| | | | 1045 | FHAINGYIMDTLPGLVMAQDQ |
| | | | 1046 | RFHAINGYIMDTLPGLVMAQD |
| | | | 1047 | YRFHAINGYIMDTLPGLVMAQ |
| | | | 1048 | NYRFHAINGYIMDTLPGLVMA |
| | | | 1049 | ENYRFHAINGYIMDTLPGLVM |
| | | | 1050 | KENYRFHAINGYIMDTLPGLV |
| | | | 1051 | FKENYRFHAINGYIMDTLPGL |
| | | | 1052 | TFKENYRFHAINGYIMDTLPG |
| | | | 1053 | PTFKENYRFHAINGYIMDTLP |
| | | | 1054 | DPTFKENYRFHAINGYIMDTL |
| | | | 1055 | EDPTFKENYRFHAINGYIMDT |
| | | | 1056 | MEDPTFKENYRFHAINGYIMD |
| | | | 1057 | QMEDPTFKENYRFHAINGYIM |
| | | | 1058 | IQMEDPTFKENYRFHAINGYI |
| | | | 1059 | NIQMEDPTFKENYRFHAINGY |
| | | | 1060 | CNIQMEDPTFKENYRFHAING |
| | | | 1061 | PCNIQMEDPTFKENYRFHAIN |
| | | | 1062 | APCNIQMEDPTFKENYRFHAI |
| | | | 1063 | RAPCNIQMEDPTFKENYRFHA |

TABLE 52

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1920 | GCA/GTA | Ala/Val | 1064 | AINGYIMDTLPGLVMAQDQRI |
| | | | 1065 | HAINGYIMDTLPGLVMAQDQR |
| | | | 1066 | FHAINGYIMDTLPGLVMAQDQ |
| | | | 1067 | RFHAINGYIMDTLPGLVMAQD |
| | | | 1068 | YRFHAINGYIMDTLPGLVMAQ |
| | | | 1069 | NYRFHAINGYIMDTLPGLVMA |
| | | | 1070 | ENYRFHAINGYIMDTLPGLVM |
| | | | 1071 | KENYRFHAINGYIMDTLPGLV |
| | | | 1072 | FKENYRFHAINGYIMDTLPGL |
| | | | 1073 | TFKENYRFHAINGYIMDTLPG |
| | | | 1074 | PTFKENYRFHAINGYIMDTLP |
| | | | 1075 | DPTFKENYRFHAINGYIMDTL |
| | | | 1076 | EDPTFKENYRFHAINGYIMDT |
| | | | 1077 | MEDPTFKENYRFHAINGYIMD |
| | | | 1078 | QMEDPTFKENYRFHAINGYIM |
| | | | 1079 | IQMEDPTFKENYRFHAINGYI |
| | | | 1080 | NIQMEDPTFKENYRFHAINGY |
| | | | 1081 | CNIQMEDPTFKENYRFHAING |
| | | | 1082 | PCNIQMEDPTFKENYRFHAIN |
| | | | 1083 | APCNIQMEDPTFKENYRFHAI |
| | | | 1084 | RAPCNIQMEDPTFKENYRFHA |

TABLE 53

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1922 | AAT/GAT | Asn/Asp | 1085 | NGYIMDTLPGLVMAQDQRIRW |
| | | | 1086 | INGYIMDTLPGLVMAQDQRIR |
| | | | 1087 | AINGYIMDTLPGLVMAQDQRI |
| | | | 1088 | HAINGYIMDTLPGLVMAQDQR |
| | | | 1089 | FHAINGYIMDTLPGLVMAQDQ |
| | | | 1090 | RFHAINGYIMDTLPGLVMAQD |
| | | | 1091 | YRFHAINGYIMDTLPGLVMAQ |
| | | | 1092 | NYRFHAINGYIMDTLPGLVMA |
| | | | 1093 | ENYRFHAINGYIMDTLPGLVM |
| | | | 1094 | KENYRFHAINGYIMDTLPGLV |
| | | | 1095 | FKENYRFHAINGYIMDTLPGL |
| | | | 1096 | TFKENYRFHAINGYIMDTLPG |
| | | | 1097 | PTFKENYRFHAINGYIMDTLP |
| | | | 1098 | DPTFKENYRFHAINGYIMDTL |
| | | | 1099 | EDPTFKENYRFHAINGYIMDT |
| | | | 1100 | MEDPTFKENYRFHAINGYIMD |
| | | | 1101 | QMEDPTFKENYRFHAINGYIM |
| | | | 1102 | IQMEDPTFKENYRFHAINGYI |
| | | | 1103 | NIQMEDPTFKENYRFHAINGY |
| | | | 1104 | CNIQMEDPTFKENYRFHAING |
| | | | 1105 | PCNIQMEDPTFKENYRFHAIN |

TABLE 54

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1923 | GGC/GAC | Gly/Asp | 1106 | GYIMDTLPGLVMAQDQRIRWY |
| | | | 1107 | NGYIMDTLPGLVMAQDQRIRW |
| | | | 1108 | INGYIMDTLPGLVMAQDQRIR |
| | | | 1109 | AINGYIMDTLPGLVMAQDQRI |
| | | | 1110 | HAINGYIMDTLPGLVMAQDQR |
| | | | 1111 | FHAINGYIMDTLPGLVMAQDQ |
| | | | 1112 | RFHAINGYIMDTLPGLVMAQD |
| | | | 1113 | YRFHAINGYIMDTLPGLVMAQ |
| | | | 1114 | NYRFHAINGYIMDTLPGLVMA |
| | | | 1115 | ENYRFHAINGYIMDTLPGLVM |
| | | | 1116 | KENYRFHAINGYIMDTLPGLV |
| | | | 1117 | FKENYRFHAINGYIMDTLPGL |
| | | | 1118 | TFKENYRFHAINGYIMDTLPG |
| | | | 1119 | PTFKENYRFHAINGYIMDTLP |
| | | | 1120 | DPTFKENYRFHAINGYIMDTL |
| | | | 1121 | EDPTFKENYRFHAINGYIMDT |
| | | | 1122 | MEDPTFKENYRFHAINGYIMD |
| | | | 1123 | QMEDPTFKENYRFHAINGYIM |
| | | | 1124 | IQMEDPTFKENYRFHAINGYI |
| | | | 1125 | NIQMEDPTFKENYRFHAINGY |
| | | | 1126 | CNIQMEDPTFKENYRFHAING |

TABLE 55

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1952 | AAC/ACC | Asn/Thr | 1127 | NIHSIHFSGHVFTVRKKEEYK |
| | | | 1128 | ENIHSIHFSGHVFTVRKKEEY |
| | | | 1129 | NENIHSIHFSGHVFTVRKKEE |
| | | | 1130 | ENENIHSIHFSGHVFTVRKKE |
| | | | 1131 | GSNENIHSIHFSGHVFTVRKK |
| | | | 1132 | MGSNENIHSIHFSGHVFTVRK |
| | | | 1133 | SMGSNENIHSIHFSGHVFTVR |
| | | | 1134 | LSMGSNENIHSIHFSGHVFTV |
| | | | 1135 | LLSMGSNENIHSIHFSGHVFT |
| | | | 1136 | YLLSMGSNENIHSIHFSGHVF |
| | | | 1137 | WYLLSMGSNENIHSIHFSGHV |
| | | | 1138 | RWYLLSMGSNENIHSIHFSGH |
| | | | 1139 | IRWYLLSMGSNENIHSIHFSG |
| | | | 1140 | RIRWYLLSMGSNENIHSIHFS |
| | | | 1141 | QRIRWYLLSMGSNENIHSIHF |
| | | | 1142 | DQRIRWYLLSMGSNENIHSIH |
| | | | 1143 | QDQRIRWYLLSMGSNENIHSI |
| | | | 1144 | AQDQRIRWYLLSMGSNENIHS |
| | | | 1145 | MAQDQRIRWYLLSMGSNENIH |
| | | | 1146 | VMAQDQRIRWYLLSMGSNENI |
| | | | 1147 | LVMAQDQRIRWYLLSMGSNEN |

TABLE 56

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1981 | GGT/GCT | Gly/Ala | 1148 | GVFETVEMLPSKAGIWRVECL |
| | | | 1149 | PGVFETVEMLPSKAGIWRVEC |
| | | | 1150 | YPGVFETVEMLPSKAGIWRVE |
| | | | 1151 | LYPGVFETVEMLPSKAGIWRV |
| | | | 1152 | NLYPGVFETVEMLPSKAGIWR |
| | | | 1153 | YNLYPGVFETVEMLPSKAGIW |
| | | | 1154 | LYNLYPGVFETVEMLPSKAGI |
| | | | 1155 | ALYNLYPGVFETVEMLPSKAG |
| | | | 1156 | MALYNLYPGVFETVEMLPSKA |
| | | | 1157 | KMALYNLYPGVFETVEMLPSK |
| | | | 1158 | YKMALYNLYPGVFETVEMLPS |
| | | | 1159 | EYKMALYNLYPGVFETVEMLP |
| | | | 1160 | EEYKMALYNLYPGVFETVEML |
| | | | 1161 | KEEYKMALYNLYPGVFETVEM |
| | | | 1162 | KKEEYKMALYNLYPGVFETVE |
| | | | 1163 | RKKEEYKMALYNLYPGVFETV |
| | | | 1164 | VRKKEEYKMALYNLYPGVFET |
| | | | 1165 | TVRKKEEYKMALYNLYPGVFE |
| | | | 1166 | FTVRKKEEYKMALYNLYPGVF |
| | | | 1167 | VFTVRKKEEYKMALYNLYPGV |
| | | | 1168 | HVFTVRKKEEYKMALYNLYPG |

TABLE 57

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1997 | CGG/CCG | Arg/Pro | 1169 | RVECLIGEHLHAGMSTLFLVY |
| | | | 1170 | WRVECLIGEHLHAGMSTLFLV |
| | | | 1171 | IWRVECLIGEHLHAGMSTLFL |
| | | | 1172 | GIWRVECLIGEHLHAGMSTLF |
| | | | 1173 | AGIWRVECLIGEHLHAGMSTL |
| | | | 1174 | KAGIWRVECLIGEHLHAGMST |
| | | | 1175 | SKAGIWRVECLIGEHLHAGMS |
| | | | 1176 | PSKAGIWRVECLIGEHLHAGM |
| | | | 1177 | LPSKAGIWRVECLIGEHLHAG |
| | | | 1178 | MLPSKAGIWRVECLIGEHLHA |
| | | | 1179 | EMLPSKAGIWRVECLIGEHLH |
| | | | 1180 | VEMLPSKAGIWRVECLIGEHL |
| | | | 1181 | TVEMLPSKAGIWRVECLIGEH |
| | | | 1182 | ETVEMLPSKAGIWRVECLIGE |
| | | | 1183 | FETVEMLPSKAGIWRVECLIG |
| | | | 1184 | VFETVEMLPSKAGIWRVECLI |
| | | | 1185 | GVFETVEMLPSKAGIWRVECL |
| | | | 1186 | PGVFETVEMLPSKAGIWRVEC |
| | | | 1187 | YPGVFETVEMLPSKAGIWRVE |
| | | | 1188 | LYPGVFETVEMLPSKAGIWRV |
| | | | 1189 | NLYPGVFETVEMLPSKAGIWR |

TABLE 58

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1997 | CGG/TGG | Arg/Trp | 1190 | RVECLIGEHLHAGMSTLFLVY |
| | | | 1191 | WRVECLIGEHLHAGMSTLFLV |
| | | | 1192 | IWRVECLIGEHLHAGMSTLFL |
| | | | 1193 | GIWRVECLIGEHLHAGMSTLF |
| | | | 1194 | AGIWRVECLIGEHLHAGMSTL |
| | | | 1195 | KAGIWRVECLIGEHLHAGMST |
| | | | 1196 | SKAGIWRVECLIGEHLHAGMS |
| | | | 1197 | PSKAGIWRVECLIGEHLHAGM |
| | | | 1198 | LPSKAGIWRVECLIGEHLHAG |
| | | | 1199 | MLPSKAGIWRVECLIGEHLHA |
| | | | 1200 | EMLPSKAGIWRVECLIGEHLH |
| | | | 1201 | VEMLPSKAGIWRVECLIGEHL |
| | | | 1202 | TVEMLPSKAGIWRVECLIGEH |
| | | | 1203 | ETVEMLPSKAGIWRVECLIGE |
| | | | 1204 | FETVEMLPSKAGIWRVECLIG |
| | | | 1205 | VFETVEMLPSKAGIWRVECLI |
| | | | 1206 | GVFETVEMLPSKAGIWRVECL |
| | | | 1207 | PGVFETVEMLPSKAGIWRVEC |
| | | | 1208 | YPGVFETVEMLPSKAGIWRVE |
| | | | 1209 | LYPGVFETVEMLPSKAGIWRV |
| | | | 1210 | NLYPGVFETVEMLPSKAGIWR |

TABLE 59

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 1999 | GAA/GGA | Glu/Gly | 1211 | ECLIGEHLHAGMSTLFLVYSN |
| | | | 1212 | VECLIGEHLHAGMSTLFLVYS |
| | | | 1213 | RVECLIGEHLHAGMSTLFLVY |
| | | | 1214 | WRVECLIGEHLHAGMSTLFLV |
| | | | 1215 | IWRVECLIGEHLHAGMSTLFL |
| | | | 1216 | GIWRVECLIGEHLHAGMSTLF |
| | | | 1217 | AGIWRVECLIGEHLHAGMSTL |
| | | | 1218 | KAGIWRVECLIGEHLHAGMST |
| | | | 1219 | SKAGIWRVECLIGEHLHAGMS |
| | | | 1220 | PSKAGIWRVECLIGEHLHAGM |
| | | | 1221 | LPSKAGIWRVECLIGEHLHAG |
| | | | 1222 | MLPSKAGIWRVECLIGEHLHA |
| | | | 1223 | EMLPSKAGIWRVECLIGEHLH |
| | | | 1224 | VEMLPSKAGIWRVECLIGEHL |
| | | | 1225 | TVEMLPSKAGIWRVECLIGEH |
| | | | 1226 | ETVEMLPSKAGIWRVECLIGE |
| | | | 1227 | FETVEMLPSKAGIWRVECLIG |
| | | | 1228 | VFETVEMLPSKAGIWRVECLI |
| | | | 1229 | GVFETVEMLPSKAGIWRVECL |
| | | | 1230 | PGVFETVEMLPSKAGIWRVEC |
| | | | 1231 | YPGVFETVEMLPSKAGIWRVE |

TABLE 60

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2004 | GAG/AAG | Glu/Lys | 1232 | EHLHAGMSTLFLVYSNKCQTP |
| | | | 1233 | GEHLHAGMSTLFLVYSNKCQT |
| | | | 1234 | IGEHLHAGMSTLFLVYSNKCQ |
| | | | 1235 | LIGEHLHAGMSTLFLVYSNKC |
| | | | 1236 | CLIGEHLHAGMSTLFLVYSNK |
| | | | 1237 | ECLIGEHLHAGMSTLFLVYSN |
| | | | 1238 | VECLIGEHLHAGMSTLFLVYS |
| | | | 1239 | RVECLIGEHLHAGMSTLFLVY |
| | | | 1240 | WRVECLIGEHLHAGMSTLFLV |
| | | | 1241 | IWRVECLIGEHLHAGMSTLFL |
| | | | 1242 | GIWRVECLIGEHLHAGMSTLF |
| | | | 1243 | AGIWRVECLIGEHLHAGMSTL |
| | | | 1244 | KAGIWRVECLIGEHLHAGMST |
| | | | 1245 | SKAGIWRVECLIGEHLHAGMS |
| | | | 1246 | PSKAGIWRVECLIGEHLHAGM |
| | | | 1247 | LPSKAGIWRVECLIGEHLHAG |
| | | | 1248 | MLPSKAGIWRVECLIGEHLHA |
| | | | 1249 | EMLPSKAGIWRVECLIGEHLH |
| | | | 1250 | VEMLPSKAGIWRVECLIGEHL |
| | | | 1251 | TVEMLPSKAGIWRVECLIGEH |
| | | | 1252 | ETVEMLPSKAGIWRVECLIGE |

TABLE 61

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2009 | GGG/AGG | Gly/Arg | 1253 | GMSTLFLVYSNKCQTPLGMAS |
| | | | 1254 | AGMSTLFLVYSNKCQTPLGMA |
| | | | 1255 | HAGMSTLFLVYSNKCQTPLGM |
| | | | 1256 | LHAGMSTLFLVYSNKCQTPLG |
| | | | 1257 | HLHAGMSTLFLVYSNKCQTPL |
| | | | 1258 | EHLHAGMSTLFLVYSNKCQTP |
| | | | 1259 | GEHLHAGMSTLFLVYSNKCQT |
| | | | 1260 | IGEHLHAGMSTLFLVYSNKCQ |
| | | | 1261 | LIGEHLHAGMSTLFLVYSNKC |
| | | | 1262 | CLIGEHLHAGMSTLFLVYSNK |
| | | | 1263 | ECLIGEHLHAGMSTLFLVYSN |
| | | | 1264 | VECLIGEHLHAGMSTLFLVYS |
| | | | 1265 | RVECLIGEHLHAGMSTLFLVY |
| | | | 1266 | WRVECLIGEHLHAGMSTLFLV |
| | | | 1267 | IWRVECLIGEHLHAGMSTLFL |
| | | | 1268 | GIWRVECLIGEHLHAGMSTLF |
| | | | 1269 | AGIWRVECLIGEHLHAGMSTL |
| | | | 1270 | KAGIWRVECLIGEHLHAGMST |
| | | | 1271 | SKAGIWRVECLIGEHLHAGMS |
| | | | 1272 | PSKAGIWRVECLIGEHLHAGM |
| | | | 1273 | LPSKAGIWRVECLIGEHLHAG |

TABLE 62

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2016 | GTG/GCG | Val/Ala | 1274 | VYSNKCQTPLGMASGHIRDFQ |
| | | | 1275 | LVYSNKCQTPLGMASGHIRDF |
| | | | 1276 | FLVYSNKCQTPLGMASGHIRD |
| | | | 1277 | LFLVYSNKCQTPLGMASGHIR |
| | | | 1278 | TLFLVYSNKCQTPLGMASGHI |
| | | | 1279 | STLFLVYSNKCQTPLGMASGH |
| | | | 1280 | MSTLFLVYSNKCQTPLGMASG |
| | | | 1281 | GMSTLFLVYSNKCQTPLGMAS |
| | | | 1282 | AGMSTLFLVYSNKCQTPLGMA |
| | | | 1283 | HAGMSTLFLVYSNKCQTPLGM |
| | | | 1284 | LHAGMSTLFLVYSNKCQTPLG |
| | | | 1285 | HLHAGMSTLFLVYSNKCQTPL |
| | | | 1286 | EHLHAGMSTLFLVYSNKCQTP |
| | | | 1287 | GEHLHAGMSTLFLVYSNKCQT |
| | | | 1288 | IGEHLHAGMSTLFLVYSNKCQ |
| | | | 1289 | LIGEHLHAGMSTLFLVYSNKC |
| | | | 1290 | CLIGEHLHAGMSTLFLVYSNK |
| | | | 1291 | ECLIGEHLHAGMSTLFLVYSN |
| | | | 1292 | VECLIGEHLHAGMSTLFLVYS |
| | | | 1293 | RVECLIGEHLHAGMSTLFLVY |
| | | | 1294 | WRVECLIGEHLHAGMSTLFLV |

TABLE 63

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2039 | GCT/CCT | Ala/Pro | 1295 | ASGQYGQWAPKLARLHYSGSI |
| | | | 1296 | TASGQYGQWAPKLARLHYSGS |
| | | | 1297 | ITASGQYGQWAPKLARLHYSG |
| | | | 1298 | QITASGQYGQWAPKLARLHYS |
| | | | 1299 | FQITASGQYGQWAPKLARLHY |
| | | | 1300 | DFQITASGQYGQWAPKLARLH |
| | | | 1301 | RDFQITASGQYGQWAPKLARL |
| | | | 1302 | IRDFQITASGQYGQWAPKLAR |
| | | | 1303 | HIRDFQITASGQYGQWAPKLA |
| | | | 1304 | GHIRDFQITASGQYGQWAPKL |
| | | | 1305 | SGHIRDFQITASGQYGQWAPK |
| | | | 1306 | ASGHIRDFQITASGQYGQWAP |
| | | | 1307 | MASGHIRDFQITASGQYGQWA |
| | | | 1308 | GMASGHIRDFQITASGQYGQW |
| | | | 1309 | LGMASGHIRDFQITASGQYGQ |
| | | | 1310 | PLGMASGHIRDFQITASGQYG |
| | | | 1311 | TPLGMASGHIRDFQITASGQY |
| | | | 1312 | QTPLGMASGHIRDFQITASGQ |
| | | | 1313 | CQTPLGMASGHIRDFQITASG |
| | | | 1314 | KCQTPLGMASGHIRDFQITAS |
| | | | 1315 | NKCQTPLGMASGHIRDFQITA |

TABLE 64

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2062 | TGG/TGC | Trp/Cys | 1316 | WSTKEPFSWIKVDLLAPMIIH |
| | | | 1317 | AWSTKEPFSWIKVDLLAPMII |
| | | | 1318 | NAWSTKEPFSWIKVDLLAPMI |
| | | | 1319 | INAWSTKEPFSWIKVDLLAPM |
| | | | 1320 | SINAWSTKEPFSWIKVDLLAP |
| | | | 1321 | GSINAWSTKEPFSWIKVDLLA |
| | | | 1322 | SGSINAWSTKEPFSWIKVDLL |
| | | | 1323 | YSGSINAWSTKEPFSWIKVDL |
| | | | 1324 | HYSGSINAWSTKEPFSWIKVD |
| | | | 1325 | LHYSGSINAWSTKEPFSWIKV |
| | | | 1326 | RLHYSGSINAWSTKEPFSWIK |
| | | | 1327 | ARLHYSGSINAWSTKEPFSWI |
| | | | 1328 | LARLHYSGSINAWSTKEPFSW |
| | | | 1329 | KLARLHYSGSINAWSTKEPFS |
| | | | 1330 | PKLARLHYSGSINAWSTKEPF |
| | | | 1331 | APKLARLHYSGSINAWSTKEP |
| | | | 1332 | WAPKLARLHYSGSINAWSTKE |
| | | | 1333 | QWAPKLARLHYSGSINAWSTK |
| | | | 1334 | GQWAPKLARLHYSGSINAWST |
| | | | 1335 | YGQWAPKLARLHYSGSINAWS |
| | | | 1336 | QYGQWAPKLARLHYSGSINAW |

TABLE 65

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2074 | GAT/GGT | Asp/Gly | 1337 | DLLAPMIIHGIKTQGARQKFS |
| | | | 1338 | VDLLAPMIIHGIKTQGARQKF |
| | | | 1339 | KVDLLAPMIIHGIKTQGARQK |
| | | | 1340 | IKVDLLAPMIIHGIKTQGARQ |
| | | | 1341 | WIKVDLLAPMIIHGIKTQGAR |
| | | | 1342 | SWIKVDLLAPMIIHGIKTQGA |
| | | | 1343 | FSWIKVDLLAPMIIHGIKTQG |
| | | | 1344 | PFSWIKVDLLAPMIIHGIKTQ |
| | | | 1345 | EPFSWIKVDLLAPMIIHGIKT |
| | | | 1346 | KEPFSWIKVDLLAPMIIHGIK |
| | | | 1347 | TKEPFSWIKVDLLAPMIIHGI |
| | | | 1348 | STKEPFSWIKVDLLAPMIIHG |
| | | | 1349 | WSTKEPFSWIKVDLLAPMIIH |
| | | | 1350 | AWSTKEPFSWIKVDLLAPMII |
| | | | 1351 | NAWSTKEPFSWIKVDLLAPMI |
| | | | 1352 | INAWSTKEPFSWIKVDLLAPM |
| | | | 1353 | SINAWSTKEPFSWIKVDLLAP |
| | | | 1354 | GSINAWSTKEPFSWIKVDLLA |
| | | | 1355 | SGSINAWSTKEPFSWIKVDLL |
| | | | 1356 | YSGSINAWSTKEPFSWIKVDL |
| | | | 1357 | HYSGSINAWSTKEPFSWIKVD |

TABLE 66

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2083 | GGC/GAC | Gly/Asp | 1358 | GIKTQGARQKFSSLYISQFII |
| | | | 1359 | HGIKTQGARQKFSSLYISQFI |
| | | | 1360 | IHGIKTQGARQKFSSLYISQF |
| | | | 1361 | IIHGIKTQGARQKFSSLYISQ |
| | | | 1362 | MIIHGIKTQGARQKFSSLYIS |
| | | | 1363 | PMIIHGIKTQGARQKFSSLYI |
| | | | 1364 | APMIIHGIKTQGARQKFSSLY |
| | | | 1365 | LAPMIIHGIKTQGARQKFSSL |
| | | | 1366 | LLAPMIIHGIKTQGARQKFSS |
| | | | 1367 | DLLAPMIIHGIKTQGARQKFS |
| | | | 1368 | VDLLAPMIIHGIKTQGARQKF |
| | | | 1369 | KVDLLAPMIIHGIKTQGARQK |
| | | | 1370 | IKVDLLAPMIIHGIKTQGARQ |
| | | | 1371 | WIKVDLLAPMIIHGIKTQGAR |
| | | | 1372 | SWIKVDLLAPMIIHGIKTQGA |
| | | | 1373 | FSWIKVDLLAPMIIHGIKTQG |
| | | | 1374 | PFSWIKVDLLAPMIIHGIKTQ |
| | | | 1375 | EPFSWIKVDLLAPMIIHGIKT |
| | | | 1376 | KEPFSWIKVDLLAPMIIHGIK |
| | | | 1377 | TKEPFSWIKVDLLAPMIIHGI |
| | | | 1378 | STKEPFSWIKVDLLAPMIIHG |

TABLE 67

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2086 | ACC/AAC | Thr/Asn | 1379 | TQGARQKFSSLYISQFIIMYS |
| | | | 1380 | KTQGARQKFSSLYISQFIIMY |
| | | | 1381 | IKTQGARQKFSSLYISQFIIM |
| | | | 1382 | GIKTQGARQKFSSLYISQFII |
| | | | 1383 | HGIKTQGARQKFSSLYISQFI |
| | | | 1384 | IHGIKTQGARQKFSSLYISQF |
| | | | 1385 | IIHGIKTQGARQKFSSLYISQ |
| | | | 1386 | MIIHGIKTQGARQKFSSLYIS |
| | | | 1387 | PMIIHGIKTQGARQKFSSLYI |
| | | | 1388 | APMIIHGIKTQGARQKFSSLY |
| | | | 1389 | LAPMIIHGIKTQGARQKFSSL |
| | | | 1390 | LLAPMIIHGIKTQGARQKFSS |
| | | | 1391 | DLLAPMIIHGIKTQGARQKFS |
| | | | 1392 | VDLLAPMIIHGIKTQGARQKF |
| | | | 1393 | KVDLLAPMIIHGIKTQGARQK |
| | | | 1394 | IKVDLLAPMIIHGIKTQGARQ |
| | | | 1395 | WIKVDLLAPMIIHGIKTQGAR |
| | | | 1396 | SWIKVDLLAPMIIHGIKTQGA |
| | | | 1397 | FSWIKVDLLAPMIIHGIKTQG |
| | | | 1398 | PFSWIKVDLLAPMIIHGIKTQ |
| | | | 1399 | EPFSWIKVDLLAPMIIHGIKT |

TABLE 68

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2105 | TAT/TGT | Tyr/Cys | 1400 | YSLDGKKWQTYRGNSTGTLMV |
| | | | 1401 | MYSLDGKKWQTYRGNSTGTLM |
| | | | 1402 | IMYSLDGKKWQTYRGNSTGTL |
| | | | 1403 | IIMYSLDGKKWQTYRGNSTGT |
| | | | 1404 | FIIMYSLDGKKWQTYRGNSTG |
| | | | 1405 | QFIIMYSLDGKKWQTYRGNST |
| | | | 1406 | SQFIIMYSLDGKKWQTYRGNS |
| | | | 1407 | ISQFIIMYSLDGKKWQTYRGN |
| | | | 1408 | YISQFIIMYSLDGKKWQTYRG |
| | | | 1409 | LYISQFIIMYSLDGKKWQTYR |
| | | | 1410 | SLYISQFIIMYSLDGKKWQTY |
| | | | 1411 | SSLYISQFIIMYSLDGKKWQT |
| | | | 1412 | FSSLYISQFIIMYSLDGKKWQ |
| | | | 1413 | KFSSLYISQFIIMYSLDGKKW |
| | | | 1414 | QKFSSLYISQFIIMYSLDGKK |
| | | | 1415 | RQKFSSLYISQFIIMYSLDGK |
| | | | 1416 | ARQKFSSLYISQFIIMYSLDG |
| | | | 1417 | GARQKFSSLYISQFIIMYSLD |
| | | | 1418 | QGARQKFSSLYISQFIIMYSL |
| | | | 1419 | TQGARQKFSSLYISQFIIMYS |
| | | | 1420 | KTQGARQKFSSLYISQFIIMY |

TABLE 69

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2129 | AAT/AGT | Asn/Ser | 1421 | NVDSSGIKHNIFNPPIIARYI |
| | | | 1422 | GNVDSSGIKHNIFNPPIIARY |
| | | | 1423 | FGNVDSSGIKHNIFNPPIIAR |
| | | | 1424 | FFGNVDSSGIKHNIFNPPIIA |
| | | | 1425 | VFFGNVDSSGIKHNIFNPPII |
| | | | 1426 | MVFFGNVDSSGIKHNIFNPPI |
| | | | 1427 | LMVFFGNVDSSGIKHNIFNPP |
| | | | 1428 | TLMVFFGNVDSSGIKHNIFNP |
| | | | 1429 | GTLMVFFGNVDSSGIKHNIFN |
| | | | 1430 | TGTLMVFFGNVDSSGIKHNIF |
| | | | 1431 | STGTLMVFFGNVDSSGIKHNI |
| | | | 1432 | NSTGTLMVFFGNVDSSGIKHN |
| | | | 1433 | GNSTGTLMVFFGNVDSSGIKH |
| | | | 1434 | RGNSTGTLMVFFGNVDSSGIK |
| | | | 1435 | YRGNSTGTLMVFFGNVDSSGI |
| | | | 1436 | TYRGNSTGTLMVFFGNVDSSG |
| | | | 1437 | QTYRGNSTGTLMVFFGNVDSS |
| | | | 1438 | WQTYRGNSTGTLMVFFGNVDS |
| | | | 1439 | KWQTYRGNSTGTLMVFFGNVD |
| | | | 1440 | KKWQTYRGNSTGTLMVFFGNV |
| | | | 1441 | GKKWQTYRGNSTGTLMVFFGN |

TABLE 70

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2150 | CGT/CAT | Arg/His | 1442 | RLHPTHYSIRSTLRMELMGCD |
| | | | 1443 | IRLHPTHYSIRSTLRMELMGC |
| | | | 1444 | YIRLHPTHYSIRSTLRMELMG |
| | | | 1445 | RYIRLHPTHYSIRSTLRMELM |
| | | | 1446 | ARYIRLHPTHYSIRSTLRMEL |
| | | | 1447 | IARYIRLHPTHYSIRSTLRME |
| | | | 1448 | IIARYIRLHPTHYSIRSTLRM |
| | | | 1449 | PIIARYIRLHPTHYSIRSTLR |
| | | | 1450 | PPIIARYIRLHPTHYSIRSTL |
| | | | 1451 | NPPIIARYIRLHPTHYSIRST |
| | | | 1452 | FNPPIIARYIRLHPTHYSIRS |
| | | | 1453 | IFNPPIIARYIRLHPTHYSIR |
| | | | 1454 | NIFNPPIIARYIRLHPTHYSI |
| | | | 1455 | HNIFNPPIIARYIRLHPTHYS |
| | | | 1456 | KHNIFNPPIIARYIRLHPTHY |
| | | | 1457 | IKHNIFNPPIIARYIRLHPTH |
| | | | 1458 | GIKHNIFNPPIIARYIRLHPT |
| | | | 1459 | SGIKHNIFNPPIIARYIRLHP |
| | | | 1460 | SSGIKHNIFNPPIIARYIRLH |
| | | | 1461 | DSSGIKHNIFNPPIIARYIRL |
| | | | 1462 | VDSSGIKHNIFNPPIIARYIR |

TABLE 71

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2159 | CGC/TGC | Arg/Cys | 1463 | RSTLRMELMGCDLNSCSMPLG |
| | | | 1464 | IRSTLRMELMGCDLNSCSMPL |
| | | | 1465 | SIRSTLRMELMGCDLNSCSMP |
| | | | 1466 | YSIRSTLRMELMGCDLNSCSM |
| | | | 1467 | HYSIRSTLRMELMGCDLNSCS |
| | | | 1468 | THYSIRSTLRMELMGCDLNSC |
| | | | 1469 | PTHYSIRSTLRMELMGCDLNS |
| | | | 1470 | HPTHYSIRSTLRMELMGCDLN |
| | | | 1471 | LHPTHYSIRSTLRMELMGCDL |
| | | | 1472 | RLHPTHYSIRSTLRMELMGCD |
| | | | 1473 | IRLHPTHYSIRSTLRMELMGC |
| | | | 1474 | YIRLHPTHYSIRSTLRMELMG |
| | | | 1475 | RYIRLHPTHYSIRSTLRMELM |
| | | | 1476 | ARYIRLHPTHYSIRSTLRMEL |
| | | | 1477 | IARYIRLHPTHYSIRSTLRME |
| | | | 1478 | IIARYIRLHPTHYSIRSTLRM |
| | | | 1479 | PIIARYIRLHPTHYSIRSTLR |
| | | | 1480 | PPIIARYIRLHPTHYSIRSTL |
| | | | 1481 | NPPIIARYIRLHPTHYSIRST |
| | | | 1482 | FNPPIIARYIRLHPTHYSIRS |
| | | | 1483 | IFNPPIIARYIRLHPTHYSIR |

TABLE 72

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2163 | CGC/CAC | Arg/His | 1484 | RMELMGCDLNSCSMPLGMESK |
| | | | 1485 | LRMELMGCDLNSCSMPLGMES |
| | | | 1486 | TLRMELMGCDLNSCSMPLGME |
| | | | 1487 | STLRMELMGCDLNSCSMPLGM |
| | | | 1488 | RSTLRMELMGCDLNSCSMPLG |
| | | | 1489 | IRSTLRMELMGCDLNSCSMPL |
| | | | 1490 | SIRSTLRMELMGCDLNSCSMP |
| | | | 1491 | YSIRSTLRMELMGCDLNSCSM |
| | | | 1492 | HYSIRSTLRMELMGCDLNSCS |
| | | | 1493 | THYSIRSTLRMELMGCDLNSC |
| | | | 1494 | PTHYSIRSTLRMELMGCDLNS |
| | | | 1495 | HPTHYSIRSTLRMELMGCDLN |
| | | | 1496 | LHPTHYSIRSTLRMELMGCDL |
| | | | 1497 | RLHPTHYSIRSTLRMELMGCD |
| | | | 1498 | IRLHPTHYSIRSTLRMELMGC |
| | | | 1499 | YIRLHPTHYSIRSTLRMELMG |
| | | | 1500 | RYIRLHPTHYSIRSTLRMELM |
| | | | 1501 | ARYIRLHPTHYSIRSTLRMEL |
| | | | 1502 | IARYIRLHPTHYSIRSTLRME |
| | | | 1503 | IIARYIRLHPTHYSIRSTLRM |
| | | | 1504 | PIIARYIRLHPTHYSIRSTLR |

TABLE 73

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2181 | GAG/GAT | Glu/Asp | 1505 | ESKAISDAQITASSYFTNMFA |
| | | | 1506 | MESKAISDAQITASSYFTNMF |
| | | | 1507 | GMESKAISDAQITASSYFTNM |
| | | | 1508 | LGMESKAISDAQITASSYFTN |
| | | | 1509 | PLGMESKAISDAQITASSYFT |
| | | | 1510 | MPLGMESKAISDAQITASSYF |
| | | | 1511 | SMPLGMESKAISDAQITASSY |
| | | | 1512 | CSMPLGMESKAISDAQITASS |
| | | | 1513 | SCSMPLGMESKAISDAQITAS |
| | | | 1514 | NSCSMPLGMESKAISDAQITA |
| | | | 1515 | LNSCSMPLGMESKAISDAQIT |
| | | | 1516 | DLNSCSMPLGMESKAISDAQI |
| | | | 1517 | CDLNSCSMPLGMESKAISDAQ |
| | | | 1518 | GCDLNSCSMPLGMESKAISDA |
| | | | 1519 | MGCDLNSCSMPLGMESKAISD |
| | | | 1520 | LMGCDLNSCSMPLGMESKAIS |
| | | | 1521 | ELMGCDLNSCSMPLGMESKAI |
| | | | 1522 | MELMGCDLNSCSMPLGMESKA |
| | | | 1523 | RMELMGCDLNSCSMPLGMESK |
| | | | 1524 | LRMELMGCDLNSCSMPLGMES |
| | | | 1525 | TLRMELMGCDLNSCSMPLGME |

TABLE 74

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2201 | GCC/CCC | Ala/Pro | 1526 | ATWSPSKARLHLQGRSNAWRP |
| | | | 1527 | FATWSPSKARLHLQGRSNAWR |
| | | | 1528 | MFATWSPSKARLHLQGRSNAW |
| | | | 1529 | NMFATWSPSKARLHLQGRSNA |
| | | | 1530 | TNMFATWSPSKARLHLQGRSN |
| | | | 1531 | FTNMFATWSPSKARLHLQGRS |
| | | | 1532 | YFTNMFATWSPSKARLHLQGR |
| | | | 1533 | SYFTNMFATWSPSKARLHLQG |
| | | | 1534 | SSYFTNMFATWSPSKARLHLQ |
| | | | 1535 | ASSYFTNMFATWSPSKARLHL |
| | | | 1536 | TASSYFTNMFATWSPSKARLH |
| | | | 1537 | ITASSYFTNMFATWSPSKARL |
| | | | 1538 | QITASSYFTNMFATWSPSKAR |
| | | | 1539 | AQITASSYFTNMFATWSPSKA |
| | | | 1540 | DAQITASSYFTNMFATWSPSK |
| | | | 1541 | SDAQITASSYFTNMFATWSPS |
| | | | 1542 | ISDAQITASSYFTNMFATWSP |
| | | | 1543 | AISDAQITASSYFTNMFATWS |
| | | | 1544 | KAISDAQITASSYFTNMFATW |
| | | | 1545 | SKAISDAQITASSYFTNMFAT |
| | | | 1546 | ESKAISDAQITASSYFTNMFA |

TABLE 75

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2209 | CGA/CAA | Arg/Gln | 1547 | RLHLQGRSNAWRPQVNNPKEW |
| | | | 1548 | ARLHLQGRSNAWRPQVNNPKE |
| | | | 1549 | KARLHLQGRSNAWRPQVNNPK |
| | | | 1550 | SKARLHLQGRSNAWRPQVNNP |
| | | | 1551 | PSKARLHLQGRSNAWRPQVNN |
| | | | 1552 | SPSKARLHLQGRSNAWRPQVN |
| | | | 1553 | WSPSKARLHLQGRSNAWRPQV |
| | | | 1554 | TWSPSKARLHLQGRSNAWRPQ |
| | | | 1555 | ATWSPSKARLHLQGRSNAWRP |
| | | | 1556 | FATWSPSKARLHLQGRSNAWR |
| | | | 1557 | MFATWSPSKARLHLQGRSNAW |
| | | | 1558 | NMFATWSPSKARLHLQGRSNA |
| | | | 1559 | TNMFATWSPSKARLHLQGRSN |
| | | | 1560 | FTNMFATWSPSKARLHLQGRS |
| | | | 1561 | YFTNMFATWSPSKARLHLQGR |
| | | | 1562 | SYFTNMFATWSPSKARLHLQG |
| | | | 1563 | SSYFTNMFATWSPSKARLHLQ |
| | | | 1564 | ASSYFTNMFATWSPSKARLHL |
| | | | 1565 | TASSYFTNMFATWSPSKARLH |
| | | | 1566 | ITASSYFTNMFATWSPSKARL |
| | | | 1567 | QITASSYFTNMFATWSPSKAR |

TABLE 76

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2218 | GCC/ACC | Ala/Thr | 1568 | AWRPQVNNPKEWLQVDFQKTM |
| | | | 1569 | NAWRPQVNNPKEWLQVDFQKT |
| | | | 1570 | SNAWRPQVNNPKEWLQVDFQK |
| | | | 1571 | RSNAWRPQVNNPKEWLQVDFQ |
| | | | 1572 | GRSNAWRPQVNNPKEWLQVDF |
| | | | 1573 | QGRSNAWRPQVNNPKEWLQVD |
| | | | 1574 | LQGRSNAWRPQVNNPKEWLQV |
| | | | 1575 | HLQGRSNAWRPQVNNPKEWLQ |
| | | | 1576 | LHLQGRSNAWRPQVNNPKEWL |
| | | | 1577 | RLHLQGRSNAWRPQVNNPKEW |
| | | | 1578 | ARLHLQGRSNAWRPQVNNPKE |
| | | | 1579 | KARLHLQGRSNAWRPQVNNPK |
| | | | 1580 | SKARLHLQGRSNAWRPQVNNP |
| | | | 1581 | PSKARLHLQGRSNAWRPQVNN |
| | | | 1582 | SPSKARLHLQGRSNAWRPQVN |
| | | | 1583 | WSPSKARLHLQGRSNAWRPQV |
| | | | 1584 | TWSPSKARLHLQGRSNAWRPQ |
| | | | 1585 | ATWSPSKARLHLQGRSNAWRP |
| | | | 1586 | FATWSPSKARLHLQGRSNAWR |
| | | | 1587 | MFATWSPSKARLHLQGRSNAW |
| | | | 1588 | NMFATWSPSKARLHLQGRSNA |

TABLE 77

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2228 | GAG/GAC | Glu/Asp | 1589 | EWLQVDFQKTMKVTGVTTQGV |
| | | | 1590 | KEWLQVDFQKTMKVTGVTTQG |
| | | | 1591 | PKEWLQVDFQKTMKVTGVTTQ |
| | | | 1592 | NPKEWLQVDFQKTMKVTGVTT |
| | | | 1593 | NNPKEWLQVDFQKTMKVTGVT |
| | | | 1594 | VNNPKEWLQVDFQKTMKVTGV |
| | | | 1595 | QVNNPKEWLQVDFQKTMKVTG |
| | | | 1596 | PQVNNPKEWLQVDFQKTMKVT |
| | | | 1597 | RPQVNNPKEWLQVDFQKTMKV |
| | | | 1598 | WRPQVNNPKEWLQVDFQKTMK |
| | | | 1599 | AWRPQVNNPKEWLQVDFQKTM |
| | | | 1600 | NAWRPQVNNPKEWLQVDFQKT |
| | | | 1601 | SNAWRPQVNNPKEWLQVDFQK |
| | | | 1602 | RSNAWRPQVNNPKEWLQVDFQ |
| | | | 1603 | GRSNAWRPQVNNPKEWLQVDF |
| | | | 1604 | QGRSNAWRPQVNNPKEWLQVD |
| | | | 1605 | LQGRSNAWRPQVNNPKEWLQV |
| | | | 1606 | HLQGRSNAWRPQVNNPKEWLQ |
| | | | 1607 | LHLQGRSNAWRPQVNNPKEWL |
| | | | 1608 | RLHLQGRSNAWRPQVNNPKEW |
| | | | 1609 | ARLHLQGRSNAWRPQVNNPKE |

TABLE 78

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2229 | TGG/TGT | Trp/Cys | 1610 | WLQVDFQKTMKVTGVTTQGVK |
| | | | 1611 | EWLQVDFQKTMKVTGVTTQGV |
| | | | 1612 | KEWLQVDFQKTMKVTGVTTQG |
| | | | 1613 | PKEWLQVDFQKTMKVTGVTTQ |
| | | | 1614 | NPKEWLQVDFQKTMKVTGVTT |
| | | | 1615 | NNPKEWLQVDFQKTMKVTGVT |
| | | | 1616 | VNNPKEWLQVDFQKTMKVTGV |
| | | | 1617 | QVNNPKEWLQVDFQKTMKVTG |
| | | | 1618 | PQVNNPKEWLQVDFQKTMKVT |
| | | | 1619 | RPQVNNPKEWLQVDFQKTMKV |
| | | | 1620 | WRPQVNNPKEWLQVDFQKTMK |
| | | | 1621 | AWRPQVNNPKEWLQVDFQKTM |
| | | | 1622 | NAWRPQVNNPKEWLQVDFQKT |
| | | | 1623 | SNAWRPQVNNPKEWLQVDFQK |
| | | | 1624 | RSNAWRPQVNNPKEWLQVDFQ |
| | | | 1625 | GRSNAWRPQVNNPKEWLQVDF |
| | | | 1626 | QGRSNAWRPQVNNPKEWLQVD |
| | | | 1627 | LQGRSNAWRPQVNNPKEWLQV |
| | | | 1628 | HLQGRSNAWRPQVNNPKEWLQ |
| | | | 1629 | LHLQGRSNAWRPQVNNPKEWL |
| | | | 1630 | RLHLQGRSNAWRPQVNNPKEW |

TABLE 79

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2230 | CTG/CGG | Leu/Arg | 1631 | LQVDFQKTMKVTGVTTQGVKS |
| | | | 1632 | WLQVDFQKTMKVTGVTTQGVK |
| | | | 1633 | EWLQVDFQKTMKVTGVTTQGV |
| | | | 1634 | KEWLQVDFQKTMKVTGVTTQG |
| | | | 1635 | PKEWLQVDFQKTMKVTGVTTQ |
| | | | 1636 | NPKEWLQVDFQKTMKVTGVTT |
| | | | 1637 | NNPKEWLQVDFQKTMKVTGVT |
| | | | 1638 | VNNPKEWLQVDFQKTMKVTGV |
| | | | 1639 | QVNNPKEWLQVDFQKTMKVTG |
| | | | 1640 | PQVNNPKEWLQVDFQKTMKVT |
| | | | 1641 | RPQVNNPKEWLQVDFQKTMKV |
| | | | 1642 | WRPQVNNPKEWLQVDFQKTMK |
| | | | 1643 | AWRPQVNNPKEWLQVDFQKTM |
| | | | 1644 | NAWRPQVNNPKEWLQVDFQKT |
| | | | 1645 | SNAWRPQVNNPKEWLQVDFQK |
| | | | 1646 | RSNAWRPQVNNPKEWLQVDFQ |
| | | | 1647 | GRSNAWRPQVNNPKEWLQVDF |
| | | | 1648 | QGRSNAWRPQVNNPKEWLQVD |
| | | | 1649 | LQGRSNAWRPQVNNPKEWLQV |
| | | | 1650 | HLQGRSNAWRPQVNNPKEWLQ |
| | | | 1651 | LHLQGRSNAWRPQVNNPKEWL |

TABLE 80

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2232 | GTG/GCG | Val/Ala | 1652 | VDFQKTMKVTGVTTQGVKSLL |
| | | | 1653 | QVDFQKTMKVTGVTTQGVKSL |
| | | | 1654 | LQVDFQKTMKVTGVTTQGVKS |
| | | | 1655 | WLQVDFQKTMKVTGVTTQGVK |
| | | | 1656 | EWLQVDFQKTMKVTGVTTQGV |
| | | | 1657 | KEWLQVDFQKTMKVTGVTTQG |
| | | | 1658 | PKEWLQVDFQKTMKVTGVTTQ |
| | | | 1659 | NPKEWLQVDFQKTMKVTGVTT |
| | | | 1660 | NNPKEWLQVDFQKTMKVTGVT |
| | | | 1661 | VNNPKEWLQVDFQKTMKVTGV |
| | | | 1662 | QVNNPKEWLQVDFQKTMKVTG |
| | | | 1663 | PQVNNPKEWLQVDFQKTMKVT |
| | | | 1664 | RPQVNNPKEWLQVDFQKTMKV |
| | | | 1665 | WRPQVNNPKEWLQVDFQKTMK |
| | | | 1666 | AWRPQVNNPKEWLQVDFQKTM |
| | | | 1667 | NAWRPQVNNPKEWLQVDFQKT |
| | | | 1668 | SNAWRPQVNNPKEWLQVDFQK |
| | | | 1669 | RSNAWRPQVNNPKEWLQVDFQ |
| | | | 1670 | GRSNAWRPQVNNPKEWLQVDF |
| | | | 1671 | QGRSNAWRPQVNNPKEWLQVD |
| | | | 1672 | LQGRSNAWRPQVNNPKEWLQV |

TABLE 81

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2246 | CAG/AAG | Gln/Lys | 1673 | QGVKSLLTSMYVKEFLISSSQ |
| | | | 1674 | TQGVKSLLTSMYVKEFLISSS |
| | | | 1675 | TTQGVKSLLTSMYVKEFLISS |
| | | | 1676 | VTTQGVKSLLTSMYVKEFLIS |
| | | | 1677 | GVTTQGVKSLLTSMYVKEFLI |
| | | | 1678 | TGVTTQGVKSLLTSMYVKEFL |
| | | | 1679 | VTGVTTQGVKSLLTSMYVKEF |
| | | | 1680 | KVTGVTTQGVKSLLTSMYVKE |
| | | | 1681 | MKVTGVTTQGVKSLLTSMYVK |
| | | | 1682 | TMKVTGVTTQGVKSLLTSMYV |
| | | | 1683 | KTMKVTGVTTQGVKSLLTSMY |
| | | | 1684 | QKTMKVTGVTTQGVKSLLTSM |
| | | | 1685 | FQKTMKVTGVTTQGVKSLLTS |
| | | | 1686 | DFQKTMKVTGVTTQGVKSLLT |
| | | | 1687 | VDFQKTMKVTGVTTQGVKSLL |
| | | | 1688 | QVDFQKTMKVTGVTTQGVKSL |
| | | | 1689 | LQVDFQKTMKVTGVTTQGVKS |
| | | | 1690 | WLQVDFQKTMKVTGVTTQGVK |
| | | | 1691 | EWLQVDFQKTMKVTGVTTQGV |
| | | | 1692 | KEWLQVDFQKTMKVTGVTTQG |
| | | | 1693 | PKEWLQVDFQKTMKVTGVTTQ |

TABLE 82

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2257 | GTG/GGG | Val/Gly | 1694 | VKEFLISSSQDGHQWTLFFQN |
| | | | 1695 | YVKEFLISSSQDGHQWTLFFQ |
| | | | 1696 | MYVKEFLISSSQDGHQWTLFF |
| | | | 1697 | SMYVKEFLISSSQDGHQWTLF |
| | | | 1698 | TSMYVKEFLISSSQDGHQWTL |
| | | | 1699 | LTSMYVKEFLISSSQDGHQWT |
| | | | 1700 | LLTSMYVKEFLISSSQDGHQW |
| | | | 1701 | SLLTSMYVKEFLISSSQDGHQ |
| | | | 1702 | KSLLTSMYVKEFLISSSQDGH |
| | | | 1703 | VKSLLTSMYVKEFLISSSQDG |
| | | | 1704 | GVKSLLTSMYVKEFLISSSQD |
| | | | 1705 | QGVKSLLTSMYVKEFLISSSQ |
| | | | 1706 | TQGVKSLLTSMYVKEFLISSS |
| | | | 1707 | TTQGVKSLLTSMYVKEFLISS |
| | | | 1708 | VTTQGVKSLLTSMYVKEFLIS |
| | | | 1709 | GVTTQGVKSLLTSMYVKEFLI |
| | | | 1710 | TGVTTQGVKSLLTSMYVKEFL |
| | | | 1711 | VTGVTTQGVKSLLTSMYVKEF |
| | | | 1712 | KVTGVTTQGVKSLLTSMYVKE |
| | | | 1713 | MKVTGVTTQGVKSLLTSMYVK |
| | | | 1714 | TMKVTGVTTQGVKSLLTSMYV |

TABLE 83

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2260 | TTC/TGC | Phe/Cys | 1715 | FLISSSQDGHQWTLFFQNGKV |
| | | | 1716 | EFLISSSQDGHQWTLFFQNGK |
| | | | 1717 | KEFLISSSQDGHQWTLFFQNG |
| | | | 1718 | VKEFLISSSQDGHQWTLFFQN |
| | | | 1719 | YVKEFLISSSQDGHQWTLFFQ |
| | | | 1720 | MYVKEFLISSSQDGHQWTLFF |
| | | | 1721 | SMYVKEFLISSSQDGHQWTLF |
| | | | 1722 | TSMYVKEFLISSSQDGHQWTL |
| | | | 1723 | LTSMYVKEFLISSSQDGHQWT |
| | | | 1724 | LLTSMYVKEFLISSSQDGHQW |
| | | | 1725 | SLLTSMYVKEFLISSSQDGHQ |
| | TTC/ATC | Phe/Ile | 1726 | KSLLTSMYVKEFLISSSQDGH |
| | | | 1727 | VKSLLTSMYVKEFLISSSQDG |
| | | | 1728 | GVKSLLTSMYVKEFLISSSQD |
| | | | 1729 | QGVKSLLTSMYVKEFLISSSQ |
| | | | 1730 | TQGVKSLLTSMYVKEFLISSS |
| | | | 1731 | TTQGVKSLLTSMYVKEFLISS |
| | | | 1732 | VTTQGVKSLLTSMYVKEFLIS |
| | | | 1733 | GVTTQGVKSLLTSMYVKEFLI |
| | | | 1734 | TGVTTQGVKSLLTSMYVKEFL |
| | | | 1735 | VTGVTTQGVKSLLTSMYVKEF |

TABLE 84

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2286 | AAT/AAG | Asn/Lys | 1736 | NQDSFTPVVNSLDPPLLTRYL |
| | | | 1737 | GNQDSFTPVVNSLDPPLLTRY |
| | | | 1738 | QGNQDSFTPVVNSLDPPLLTR |
| | | | 1739 | FQGNQDSFTPVVNSLDPPLLT |
| | | | 1740 | VFQGNQDSFTPVVNSLDPPLL |
| | | | 1741 | KVFQGNQDSFTPVVNSLDPPL |
| | | | 1742 | VKVFQGNQDSFTPVVNSLDPP |
| | | | 1743 | KVKVFQGNQDSFTPVVNSLDP |
| | | | 1744 | GKVKVFQGNQDSFTPVVNSLD |
| | | | 1745 | NGKVKVFQGNQDSFTPVVNSL |
| | | | 1746 | QNGKVKVFQGNQDSFTPVVNS |
| | | | 1747 | FQNGKVKVFQGNQDSFTPVVN |
| | | | 1748 | FFQNGKVKVFQGNQDSFTPVV |
| | | | 1749 | LFFQNGKVKVFQGNQDSFTPV |
| | | | 1750 | TLFFQNGKVKVFQGNQDSFTP |
| | | | 1751 | WTLFFQNGKVKVFQGNQDSFT |
| | | | 1752 | QWTLFFQNGKVKVFQGNQDSF |
| | | | 1753 | HQWTLFFQNGKVKVFQGNQDS |
| | | | 1754 | GHQWTLFFQNGKVKVFQGNQD |
| | | | 1755 | DGHQWTLFFQNGKVKVFQGNQ |
| | | | 1756 | QDGHQWTLFFQNGKVKVFQGN |

TABLE 85

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2300 | CCG/CTG | Pro/Leu | 1757 | PLLTRYLRIHPQSWVHQIALR |
| | | | 1758 | PPLLTRYLRIHPQSWVHQIAL |
| | | | 1759 | DPPLLTRYLRIHPQSWVHQIA |
| | | | 1760 | LDPPLLTRYLRIHPQSWVHQI |
| | | | 1761 | SLDPPLLTRYLRIHPQSWVHQ |
| | | | 1762 | NSLDPPLLTRYLRIHPQSWVH |
| | | | 1763 | VNSLDPPLLTRYLRIHPQSWV |
| | | | 1764 | VVNSLDPPLLTRYLRIHPQSW |
| | | | 1765 | PVVNSLDPPLLTRYLRIHPQS |
| | | | 1766 | TPVVNSLDPPLLTRYLRIHPQ |
| | | | 1767 | FTPVVNSLDPPLLTRYLRIHP |
| | | | 1768 | SFTPVVNSLDPPLLTRYLRIH |
| | | | 1769 | DSFTPVVNSLDPPLLTRYLRI |
| | | | 1770 | QDSFTPVVNSLDPPLLTRYLR |
| | | | 1771 | NQDSFTPVVNSLDPPLLTRYL |
| | | | 1772 | GNQDSFTPVVNSLDPPLLTRY |
| | | | 1773 | QGNQDSFTPVVNSLDPPLLTR |
| | | | 1774 | FQGNQDSFTPVVNSLDPPLLT |
| | | | 1775 | VFQGNQDSFTPVVNSLDPPLL |
| | | | 1776 | KVFQGNQDSFTPVVNSLDPPL |
| | | | 1777 | VKVFQGNQDSFTPVVNSLDPP |

TABLE 86

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2304 | CGC/TGC | Arg/Cys | 1778 | RYLRIHPQSWVHQIALRMEVL |
| | | | 1779 | TRYLRIHPQSWVHQIALRMEV |
| | | | 1780 | LTRYLRIHPQSWVHQIALRME |
| | | | 1781 | LLTRYLRIHPQSWVHQIALRM |
| | | | 1782 | PLLTRYLRIHPQSWVHQIALR |
| | | | 1783 | PPLLTRYLRIHPQSWVHQIAL |
| | | | 1784 | DPPLLTRYLRIHPQSWVHQIA |
| | | | 1785 | LDPPLLTRYLRIHPQSWVHQI |
| | | | 1786 | SLDPPLLTRYLRIHPQSWVHQ |
| | | | 1787 | NSLDPPLLTRYLRIHPQSWVH |
| | | | 1788 | VNSLDPPLLTRYLRIHPQSWV |
| | | | 1789 | VVNSLDPPLLTRYLRIHPQSW |
| | | | 1790 | PVVNSLDPPLLTRYLRIHPQS |
| | | | 1791 | TPVVNSLDPPLLTRYLRIHPQ |
| | | | 1792 | FTPVVNSLDPPLLTRYLRIHP |
| | | | 1793 | SFTPVVNSLDPPLLTRYLRIH |
| | | | 1794 | DSFTPVVNSLDPPLLTRYLRI |
| | | | 1795 | QDSFTPVVNSLDPPLLTRYLR |
| | | | 1796 | NQDSFTPVVNSLDPPLLTRYL |
| | | | 1797 | GNQDSFTPVVNSLDPPLLTRY |
| | | | 1798 | QGNQDSFTPVVNSLDPPLLTR |

TABLE 87

| | Missense | | | |
|---|---|---|---|---|
| Reference Locus | Nucleotide | FVIIIrp/sFVIII | SEQ | |
| Position within | Change | amino acid | ID | |
| FVIIIrp | (wt/sFVIII) | difference | NO: | TIP Set |
| 2307 | CGA/CAA | Arg/Gln | 1799 | RIHPQSWVHQIALRMEVLGCE |
| | | | 1800 | LRIHPQSWVHQIALRMEVLGC |
| | | | 1801 | YLRIHPQSWVHQIALRMEVLG |
| | | | 1802 | RYLRIHPQSWVHQIALRMEVL |
| | | | 1803 | TRYLRIHPQSWVHQIALRMEV |
| | | | 1804 | LTRYLRIHPQSWVHQIALRME |
| | | | 1805 | LLTRYLRIHPQSWVHQIALRM |
| | | | 1806 | PLLTRYLRIHPQSWVHQIALR |
| | | | 1807 | PPLLTRYLRIHPQSWVHQIAL |
| | | | 1808 | DPPLLTRYLRIHPQSWVHQIA |
| | | | 1809 | LDPPLLTRYLRIHPQSWVHQI |
| | | | 1810 | SLDPPLLTRYLRIHPQSWVHQ |
| | | | 1811 | NSLDPPLLTRYLRIHPQSWVH |
| | | | 1812 | VNSLDPPLLTRYLRIHPQSWV |
| | | | 1813 | VVNSLDPPLLTRYLRIHPQSW |
| | | | 1814 | PVVNSLDPPLLTRYLRIHPQS |
| | | | 1815 | TPVVNSLDPPLLTRYLRIHPQ |
| | | | 1816 | FTPVVNSLDPPLLTRYLRIHP |
| | | | 1817 | SFTPVVNSLDPPLLTRYLRIH |
| | | | 1818 | DSFTPVVNSLDPPLLTRYLRI |
| | | | 1819 | QDSFTPVVNSLDPPLLTRYLR |

In certain aspects of the present invention, compositions directed to specific TIPs and TIP sets described in Tables 88-101, and methods using the compositions thereof, are provided herein. In one embodiment, at least one or more TIPs comprising at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, or at least 21 amino acids, including at least one reference locus based on a nsSNP, identified in Tables 88-101 are provided. In one embodiment, at least one TIP set comprising at least 9 peptides, at least 10 peptides, at least 11 peptides, at least 12 peptides, at least 13 peptides, at 14 peptides, 15 peptides, at least 16 peptides, at least 17 peptides, at least 18 peptides, at least 19 peptides, at least 20 peptides, or at least 21 peptides, wherein the first peptide of the set comprises a reference locus at its first amino acid position, the second peptide of the set comprises a reference locus at its second amino acid position, and each successive peptide in the set comprises a reference locus at an amino acid position frame-shifted one position downstream from the reference locus position of the preceding peptide, and wherein the last peptide of the set has a reference locus in its last amino acid position, wherein the TIP sets are generated from the TIPs identified in Tables 88-101 (reference locus underlined and bolded), are provided herein. Tables 88-101 are provided below.

TABLE 88

| | ns-SNP TIPs | | | |
|---|---|---|---|---|
| | Major allele | | Minor allele | |
| | SEQ | | SEQ | |
| | ID | | ID | |
| F8 ns-SNPs | NO: | E113 | NO: | D113 |
| E113D | 1820 | EYDDQTSQREKEDDKVFPGGS | 1841 | DYDDQTSQREKEDDKVFPGGS |
| | 1821 | AEYDDQTSQREKEDDKVFPGG | 1842 | ADYDDQTSQREKEDDKVFPGG |
| | 1822 | GAEYDDQTSQREKEDDKVFPG | 1843 | GADYDDQTSQREKEDDKVFPG |
| | 1823 | EGAEYDDQTSQREKEDDKVFP | 1844 | EGADYDDQTSQREKEDDKVFP |
| | 1824 | SEGAEYDDQTSQREKEDDKVF | 1845 | SEGADYDDQTSQREKEDDKVF |
| | 1825 | ASEGAEYDDQTSQREKEDDKV | 1846 | ASEGADYDDQTSQREKEDDKV |
| | 1826 | KASEGAEYDDQTSQREKEDDK | 1847 | KASEGADYDDQTSQREKEDDK |
| | 1827 | WKASEGAEYDDQTSQREKEDD | 1848 | WKASEGADYDDQTSQREKEDD |
| | 1828 | YWKASEGAEYDDQTSQREKED | 1849 | YWKASEGADYDDQTSQREKED |
| | 1829 | SYWKASEGAEYDDQTSQREKE | 1850 | SYWKASEGADYDDQTSQREKE |
| | 1830 | VSYWKASEGAEYDDQTSQREK | 1851 | VSYWKASEGADYDDQTSQREK |
| | 1831 | GVSYWKASEGAEYDDQTSQRE | 1852 | GVSYWKASEGADYDDQTSQRE |
| | 1832 | VGVSYWKASEGAEYDDQTSQR | 1853 | VGVSYWKASEGADYDDQTSQR |
| | 1833 | AVGVSYWKASEGAEYDDQTSQ | 1854 | AVGVSYWKASEGADYDDQTSQ |
| | 1834 | HAVGVSYWKASEGAEYDDQTS | 1855 | HAVGVSYWKASEGADYDDQTS |
| | 1835 | LHAVGVSYWKASEGAEYDDQT | 1856 | LHAVGVSYWKASEGADYDDQT |
| | 1836 | SLHAVGVSYWKASEGAEYDDQ | 1857 | SLHAVGVSYWKASEGADYDDQ |
| | 1837 | VSLHAVGVSYWKASEGAEYDD | 1858 | VSLHAVGVSYWKASEGADYDD |
| | 1838 | PVSLHAVGVSYWKASEGAEYD | 1859 | PVSLHAVGVSYWKASEGADYD |
| | 1839 | HPVSLHAVGVSYWKASEGAEY | 1860 | HPVSLHAVGVSYWKASEGADY |
| | 1840 | SHPVSLHAVGVSYWKASEGAE | 1861 | SHPVSLHAVGVSYWKASEGAD |

TABLE 89

| | ns-SNP TIPs | | | |
|---|---|---|---|---|
| | Major allele | | Minor allele | |
| | SEQ | | SEQ | |
| | ID | | ID | |
| F8 ns-SNPs | NO: | Q334 | NO: | P334 |
| Q334P | 1862 | QLRMKNNEEAEDYDDDLTDSE | 1883 | PLRMKNNEEAEDYDDDLTDSE |
| | 1863 | PQLRMKNNEEAEDYDDDLTDS | 1884 | PPLRMKNNEEAEDYDDDLTDS |
| | 1864 | EPQLRMKNNEEAEDYDDDLTD | 1885 | EPPLRMKNNEEAEDYDDDLTD |
| | 1865 | EEPQLRMKNNEEAEDYDDDLT | 1886 | EEPPLRMKNNEEAEDYDDDLT |
| | 1866 | PEEPQLRMKNNEEAEDYDDDL | 1887 | PEEPPLRMKNNEEAEDYDDDL |
| | 1867 | CPEEPQLRMKNNEEAEDYDDD | 1888 | CPEEPPLRMKNNEEAEDYDDD |
| | 1868 | SCPEEPQLRMKNNEEAEDYDD | 1889 | SCPEEPPLRMKNNEEAEDYDD |
| | 1869 | DSCPEEPQLRMKNNEEAEDYD | 1890 | DSCPEEPPLRMKNNEEAEDYD |
| | 1870 | VDSCPEEPQLRMKNNEEAEDY | 1891 | VDSCPEEPPLRMKNNEEAEDY |
| | 1871 | KVDSCPEEPQLRMKNNEEAED | 1892 | KVDSCPEEPPLRMKNNEEAED |
| | 1872 | VKVDSCPEEPQLRMKNNEEAE | 1893 | VKVDSCPEEPPLRMKNNEEAE |
| | 1873 | YVKVDSCPEEPQLRMKNNEEA | 1894 | YVKVDSCPEEPPLRMKNNEEA |
| | 1874 | AYVKVDSCPEEPQLRMKNNEE | 1895 | AYVKVDSCPEEPPLRMKNNEE |
| | 1875 | EAYVKVDSCPEEPQLRMKNNE | 1896 | EAYVKVDSCPEEPPLRMKNNE |
| | 1876 | MEAYVKVDSCPEEPQLRMKNN | 1897 | MEAYVKVDSCPEEPPLRMKNN |
| | 1877 | GMEAYVKVDSCPEEPQLRMKN | 1898 | GMEAYVKVDSCPEEPPLRMKN |
| | 1878 | DGMEAYVKVDSCPEEPQLRMK | 1899 | DGMEAYVKVDSCPEEPPLRMK |
| | 1879 | HDGMEAYVKVDSCPEEPQLRM | 1900 | HDGMEAYVKVDSCPEEPPLRM |
| | 1880 | QHDGMEAYVKVDSCPEEPQLR | 1901 | QHDGMEAYVKVDSCPEEPPLR |
| | 1881 | HQHDGMEAYVKVDSCPEEPQL | 1902 | HQHDGMEAYVKVDSCPEEPPL |
| | 1882 | SHQHDGMEAYVKVDSCPEEPQ | 1903 | SHQHDGMEAYVKVDSCPEEPP |

TABLE 90

| | ns-SNP TIPs | | | |
|---|---|---|---|---|
| | Major allele | | Minor allele | |
| | SEQ | | SEQ | |
| | ID | | ID | |
| F8 ns-SNPs | NO: | A387 | NO: | T387 |
| A387T | 1904 | AAEEEDWDYAPLVLAPDDRSY | 1925 | TAEEEDWDYAPLVLAPDDRSY |
| | 1905 | IAAEEEDWDYAPLVLAPDDRS | 1926 | ITAEEEDWDYAPLVLAPDDRS |
| | 1906 | YIAAEEEDWDYAPLVLAPDDR | 1927 | YITAEEEDWDYAPLVLAPDDR |
| | 1907 | HYIAAEEEDWDYAPLVLAPDD | 1928 | HYITAEEEDWDYAPLVLAPDD |
| | 1908 | VHYIAAEEEDWDYAPLVLAPD | 1929 | VHYITAEEEDWDYAPLVLAPD |
| | 1909 | WVHYIAAEEEDWDYAPLVLAP | 1930 | WVHYITAEEEDWDYAPLVLAP |
| | 1910 | TWVHYIAAEEEDWDYAPLVLA | 1931 | TWVHYITAEEEDWDYAPLVLA |
| | 1911 | KTWVHYIAAEEEDWDYAPLVL | 1932 | KTWVHYITAEEEDWDYAPLVL |
| | 1912 | PKTWVHYIAAEEEDWDYAPLV | 1933 | PKTWVHYITAEEEDWDYAPLV |
| | 1913 | HPKTWVHYIAAEEEDWDYAPL | 1934 | HPKTWVHYITAEEEDWDYAPL |
| | 1914 | KHPKTWVHYIAAEEEDWDYAP | 1935 | KHPKTWVHYITAEEEDWDYAP |
| | 1915 | KKHPKTWVHYIAAEEEDWDYA | 1936 | KKHPKTWVHYITAEEEDWDYA |
| | 1916 | AKKHPKTWVHYIAAEEEDWDY | 1937 | AKKHPKTWVHYITAEEEDWDY |
| | 1917 | VAKKHPKTWVHYIAAEEEDWD | 1938 | VAKKHPKTWVHYITAEEEDWD |
| | 1918 | SVAKKHPKTWVHYIAAEEEDW | 1939 | SVAKKHPKTWVHYITAEEEDW |
| | 1919 | RSVAKKHPKTWVHYIAAEEED | 1940 | RSVAKKHPKTWVHYITAEEED |
| | 1920 | IRSVAKKHPKTWVHYIAAEEE | 1941 | IRSVAKKHPKTWVHYITAEEE |
| | 1921 | QIRSVAKKHPKTWVHYIAAEE | 1942 | QIRSVAKKHPKTWVHYITAEE |
| | 1922 | IQIRSVAKKHPKTWVHYIAAE | 1943 | IQIRSVAKKHPKTWVHYITAE |
| | 1923 | FIQIRSVAKKHPKTWVHYIAA | 1944 | FIQIRSVAKKHPKTWVHYITA |
| | 1924 | SFIQIRSVAKKHPKTWVHYIA | 1945 | SFIQIRSVAKKHPKTWVHYIT |

TABLE 91

| | ns-SNP TIPs | | | |
|---|---|---|---|---|
| | Major allele | | Minor allele | |
| | SEQ | | SEQ | |
| | ID | | ID | |
| F8 ns-SNPs | NO: | R484 | NO: | H484 |
| R484H | 1946 | RPLYSRRLPKGVKHLKDFPIL | 1967 | HPLYSRRLPKGVKHLKDFPIL |
| | 1947 | VRPLYSRRLPKGVKHLKDFPI | 1968 | VHPLYSRRLPKGVKHLKDFPI |
| | 1948 | DVRPLYSRRLPKGVKHLKDFP | 1969 | DVHPLYSRRLPKGVKHLKDFP |
| | 1949 | TDVRPLYSRRLPKGVKHLKDF | 1970 | TDVHPLYSRRLPKGVKHLKDF |
| | 1950 | ITDVRPLYSRRLPKGVKHLKD | 1971 | ITDVHPLYSRRLPKGVKHLKD |
| | 1951 | GITDVRPLYSRRLPKGVKHLK | 1972 | GITDVHPLYSRRLPKGVKHLK |
| | 1952 | HGITDVRPLYSRRLPKGVKHL | 1973 | HGITDVHPLYSRRLPKGVKHL |
| | 1953 | PHGITDVRPLYSRRLPKGVKH | 1974 | PHGITDVHPLYSRRLPKGVKH |
| | 1954 | YPHGITDVRPLYSRRLPKGVK | 1975 | YPHGITDVHPLYSRRLPKGVK |
| | 1955 | IYPHGITDVRPLYSRRLPKGV | 1976 | IYPHGITDVHPLYSRRLPKGV |
| | 1956 | NIYPHGITDVRPLYSRRLPKG | 1977 | NIYPHGITDVHPLYSRRLPKG |
| | 1957 | YNIYPHGITDVRPLYSRRLPK | 1978 | YNIYPHGITDVHPLYSRRLPK |
| | 1958 | PYNIYPHGITDVRPLYSRRLP | 1979 | PYNIYPHGITDVHPLYSRRLP |
| | 1959 | RPYNIYPHGITDVRPLYSRRL | 1980 | RPYNIYPHGITDVHPLYSRRL |
| | 1960 | SRPYNIYPHGITDVRPLYSRR | 1981 | SRPYNIYPHGITDVHPLYSRR |
| | 1961 | ASRPYNIYPHGITDVRPLYSR | 1982 | ASRPYNIYPHGITDVHPLYSR |
| | 1962 | QASRPYNIYPHGITDVRPLYS | 1983 | QASRPYNIYPHGITDVHPLYS |
| | 1963 | NQASRPYNIYPHGITDVRPLY | 1984 | NQASRPYNIYPHGITDVHPLY |
| | 1964 | KNQASRPYNIYPHGITDVRPL | 1985 | KNQASRPYNIYPHGITDVHPL |
| | 1965 | FKNQASRPYNIYPHGITDVRP | 1986 | FKNQASRPYNIYPHGITDVHP |
| | 1966 | IFKNQASRPYNIYPHGITDVR | 1987 | IFKNQASRPYNIYPHGITDVH |

TABLE 92

| | ns-SNP TIPs | | | |
|---|---|---|---|---|
| | Major allele | | Minor allele | |
| | SEQ | | SEQ | |
| | ID | | ID | |
| F8 ns-SNPs | NO: | R776G | NO: | R776G |
| R776G | 1988 | RTPMPKIQNVSSSDLLMLLRQ | 2009 | GTPMPKIQNVSSSDLLMLLRQ |
| | 1989 | HRTPMPKIQNVSSSDLLMLLR | 2010 | HGTPMPKIQNVSSSDLLMLLR |
| | 1990 | AHRTPMPKIQNVSSSDLLMLL | 2011 | AHGTPMPKIQNVSSSDLLMLL |
| | 1991 | FAHRTPMPKIQNVSSSDLLML | 2012 | FAHGTPMPKIQNVSSSDLLML |
| | 1992 | WFAHRTPMPKIQNVSSSDLLM | 2013 | WFAHGTPMPKIQNVSSSDLLM |
| | 1993 | PWFAHRTPMPKIQNVSSSDLL | 2014 | PWFAHGTPMPKIQNVSSSDLL |
| | 1994 | DPWFAHRTPMPKIQNVSSSDL | 2015 | DPWFAHGTPMPKIQNVSSSDL |
| | 1995 | TDPWFAHRTPMPKIQNVSSSD | 2016 | TDPWFAHGTPMPKIQNVSSSD |
| | 1996 | KTDPWFAHRTPMPKIQNVSSS | 2017 | KTDPWFAHGTPMPKIQNVSSS |
| | 1997 | EKTDPWFAHRTPMPKIQNVSS | 2018 | EKTDPWFAHGTPMPKIQNVSS |
| | 1998 | IEKTDPWFAHRTPMPKIQNVS | 2019 | IEKTDPWFAHGTPMPKIQNVS |
| | 1999 | DIEKTDPWFAHRTPMPKIQNV | 2020 | DIEKTDPWFAHGTPMPKIQNV |
| | 2000 | NDIEKTDPWFAHRTPMPKIQN | 2021 | NDIEKTDPWFAHGTPMPKIQN |
| | 2001 | ENDIEKTDPWFAHRTPMPKIQ | 2022 | ENDIEKTDPWFAHGTPMPKIQ |
| | 2002 | PENDIEKTDPWFAHRTPMPKI | 2023 | PENDIEKTDPWFAHGTPMPKI |
| | 2003 | IPENDIEKTDPWFAHRTPMPK | 2024 | IPENDIEKTDPWFAHGTPMPK |
| | 2004 | TIPENDIEKTDPWFAHRTPMP | 2025 | TIPENDIEKTDPWFAHGTPMP |
| | 2005 | TTIPENDIEKTDPWFAHRTPM | 2026 | TTIPENDIEKTDPWFAHGTPM |
| | 2006 | ATTIPENDIEKTDPWFAHRTP | 2027 | ATTIPENDIEKTDPWFAHGTP |
| | 2007 | NATTIPENDIEKTDPWFAHRT | 2028 | NATTIPENDIEKTDPWFAHGT |
| | 2008 | FNATTIPENDIEKTDPWFAHR | 2029 | FNATTIPENDIEKTDPWFAHG |

TABLE 93

| | ns-SNP TIPs | | | |
|---|---|---|---|---|
| | Major allele | | Minor allele | |
| | SEQ | | SEQ | |
| | ID | | ID | |
| F8 ns-SNPs | NO: | R1107 | NO: | W1107 |
| R1107W | 2030 | RWIQRTHGKNSLNSGQGPSPK | 2051 | WWIQRTHGKNSLNSGQGPSPK |
| | 2031 | ARWIQRTHGKNSLNSGQGPSP | 2052 | AWWIQRTHGKNSLNSGQGPSP |
| | 2032 | SARWIQRTHGKNSLNSGQGPS | 2053 | SAWWIQRTHGKNSLNSGQGPS |
| | 2033 | ESARWIQRTHGKNSLNSGQGP | 2054 | ESAWWIQRTHGKNSLNSGQGP |
| | 2034 | PESARWIQRTHGKNSLNSGQG | 2055 | PESAWWIQRTHGKNSLNSGQG |
| | 2035 | LPESARWIQRTHGKNSLNSGQ | 2056 | LPESAWWIQRTHGKNSLNSGQ |
| | 2036 | FLPESARWIQRTHGKNSLNSG | 2057 | FLPESAWWIQRTHGKNSLNSG |
| | 2037 | LFLPESARWIQRTHGKNSLNS | 2058 | LFLPESAWWIQRTHGKNSLNS |
| | 2038 | MLFLPESARWIQRTHGKNSLN | 2059 | MLFLPESAWWIQRTHGKNSLN |
| | 2039 | KMLFLPESARWIQRTHGKNSL | 2060 | KMLFLPESAWWIQRTHGKNSL |
| | 2040 | FKMLFLPESARWIQRTHGKNS | 2061 | FKMLFLPESAWWIQRTHGKNS |
| | 2041 | FFKMLFLPESARWIQRTHGKN | 2062 | FFKMLFLPESAWWIQRTHGKN |
| | 2042 | SFFKMLFLPESARWIQRTHGK | 2063 | SFFKMLFLPESAWWIQRTHGK |
| | 2043 | MSFFKMLFLPESARWIQRTHG | 2064 | MSFFKMLFLPESAWWIQRTHG |
| | 2044 | DMSFFKMLFLPESARWIQRTH | 2065 | DMSFFKMLFLPESAWWIQRTH |
| | 2045 | PDMSFFKMLFLPESARWIQRT | 2066 | PDMSFFKMLFLPESAWWIQRT |
| | 2046 | NPDMSFFKMLFLPESARWIQR | 2067 | NPDMSFFKMLFLPESAWWIQR |
| | 2047 | QNPDMSFFKMLFLPESARWIQ | 2068 | QNPDMSFFKMLFLPESAWWIQ |
| | 2048 | AQNPDMSFFKMLFLPESARWI | 2069 | AQNPDMSFFKMLFLPESAWWI |
| | 2049 | DAQNPDMSFFKMLFLPESARW | 2070 | DAQNPDMSFFKMLFLPESAWW |
| | 2050 | PDAQNPDMSFFKMLFLPESAR | 2071 | PDAQNPDMSFFKMLFLPESAW |

TABLE 94

| | ns-SNP TIPs | | | |
|---|---|---|---|---|
| | Major allele | | Minor allele | |
| | SEQ | | SEQ | |
| | ID | | ID | |
| F8 ns-SNPs | NO: | D1241E | NO: | D1241E |
| D1241E | 2072 | DGAYAPVLQDFRSLNDSTNRT | 2093 | EGAYAPVLQDFRSLNDSTNRT |
| | 2073 | YDGAYAPVLQDFRSLNDSTNR | 2094 | YEGAYAPVLQDFRSLNDSTNR |
| | 2074 | SYDGAYAPVLQDFRSLNDSTN | 2095 | SYEGAYAPVLQDFRSLNDSTN |
| | 2075 | GSYDGAYAPVLQDFRSLNDST | 2096 | GSYEGAYAPVLQDFRSLNDST |
| | 2076 | EGSYDGAYAPVLQDFRSLNDS | 2097 | EGSYEGAYAPVLQDFRSLNDS |
| | 2077 | VEGSYDGAYAPVLQDFRSLND | 2098 | VEGSYEGAYAPVLQDFRSLND |
| | 2078 | NVEGSYDGAYAPVLQDFRSLN | 2099 | NVEGSYEGAYAPVLQDFRSLN |
| | 2079 | QNVEGSYDGAYAPVLQDFRSL | 2100 | QNVEGSYEGAYAPVLQDFRSL |
| | 2080 | RQNVEGSYDGAYAPVLQDFRS | 2101 | RQNVEGSYEGAYAPVLQDFRS |
| | 2081 | TRQNVEGSYDGAYAPVLQDFR | 2102 | TRQNVEGSYEGAYAPVLQDFR |
| | 2082 | STRQNVEGSYDGAYAPVLQDF | 2103 | STRQNVEGSYEGAYAPVLQDF |
| | 2083 | LSTRQNVEGSYDGAYAPVLQD | 2104 | LSTRQNVEGSYEGAYAPVLQD |
| | 2084 | LLSTRQNVEGSYDGAYAPVLQ | 2105 | LLSTRQNVEGSYEGAYAPVLQ |
| | 2085 | FLLSTRQNVEGSYDGAYAPVL | 2106 | FLLSTRQNVEGSYEGAYAPVL |
| | 2086 | LFLLSTRQNVEGSYDGAYAPV | 2107 | LFLLSTRQNVEGSYEGAYAPV |
| | 2087 | NLFLLSTRQNVEGSYDGAYAP | 2108 | NLFLLSTRQNVEGSYEGAYAP |
| | 2088 | KNLFLLSTRQNVEGSYDGAYA | 2109 | KNLFLLSTRQNVEGSYEGAYA |
| | 2089 | MKNLFLLSTRQNVEGSYDGAY | 2110 | MKNLFLLSTRQNVEGSYEGAY |
| | 2090 | FMKNLFLLSTRQNVEGSYDGA | 2111 | FMKNLFLLSTRQNVEGSYEGA |
| | 2091 | NFMKNLFLLSTRQNVEGSYDG | 2112 | NFMKNLFLLSTRQNVEGSYEG |
| | 2092 | KNFMKNLFLLSTRQNVEGSYD | 2113 | KNFMKNLFLLSTRQNVEGSYE |

TABLE 95

| | ns-SNP TIPs | | | |
|---|---|---|---|---|
| | Major allele | | Minor allele | |
| | SEQ | | SEQ | |
| | ID | | ID | |
| F8 ns-SNPs | NO: | R1260K | NO: | R1260K |
| R1260K | 2114 | RTKKHTAHFSKKGEEENLEGL | 2135 | KTKKHTAHFSKKGEEENLEGL |
| | 2115 | NRTKKHTAHFSKKGEEENLEG | 2136 | NKTKKHTAHFSKKGEEENLEG |
| | 2116 | TNRTKKHTAHFSKKGEEENLE | 2137 | TNKTKKHTAHFSKKGEEENLE |
| | 2117 | STNRTKKHTAHFSKKGEEENL | 2138 | STNKTKKHTAHFSKKGEEENL |
| | 2118 | DSTNRTKKHTAHFSKKGEEEN | 2139 | DSTNKTKKHTAHFSKKGEEEN |
| | 2119 | NDSTNRTKKHTAHFSKKGEEE | 2140 | NDSTNKTKKHTAHFSKKGEEE |
| | 2120 | LNDSTNRTKKHTAHFSKKGEE | 2141 | LNDSTNKTKKHTAHFSKKGEE |
| | 2121 | SLNDSTNRTKKHTAHFSKKGE | 2142 | SLNDSTNKTKKHTAHFSKKGE |
| | 2122 | RSLNDSTNRTKKHTAHFSKKG | 2143 | RSLNDSTNKTKKHTAHFSKKG |
| | 2123 | FRSLNDSTNRTKKHTAHFSKK | 2144 | FRSLNDSTNKTKKHTAHFSKK |
| | 2124 | DFRSLNDSTNRTKKHTAHFSK | 2145 | DFRSLNDSTNKTKKHTAHFSK |
| | 2125 | QDFRSLNDSTNRTKKHTAHFS | 2146 | QDFRSLNDSTNKTKKHTAHFS |
| | 2126 | LQDFRSLNDSTNRTKKHTAHF | 2147 | LQDFRSLNDSTNKTKKHTAHF |
| | 2127 | VLQDFRSLNDSTNRTKKHTAH | 2148 | VLQDFRSLNDSTNKTKKHTAH |
| | 2128 | PVLQDFRSLNDSTNRTKKHTA | 2149 | PVLQDFRSLNDSTNKTKKHTA |
| | 2129 | APVLQDFRSLNDSTNRTKKHT | 2150 | APVLQDFRSLNDSTNKTKKHT |
| | 2130 | YAPVLQDFRSLNDSTNRTKKH | 2151 | YAPVLQDFRSLNDSTNKTKKH |
| | 2131 | AYAPVLQDFRSLNDSTNRTKK | 2152 | AYAPVLQDFRSLNDSTNKTKK |
| | 2132 | GAYAPVLQDFRSLNDSTNRTK | 2153 | GAYAPVLQDFRSLNDSTNKTK |
| | 2133 | DGAYAPVLQDFRSLNDSTNRT | 2154 | DGAYAPVLQDFRSLNDSTNKT |
| | 2134 | YDGAYAPVLQDFRSLNDSTNR | 2155 | YDGAYAPVLQDFRSLNDSTNK |

TABLE 96

| | ns-SNP TIPs | | | |
|---|---|---|---|---|
| | Major allele | | Minor allele | |
| | SEQ | | SEQ | |
| | ID | | ID | |
| F8 ns-SNPs | NO: | L1462 | NO: | P1462 |
| L1462P | 2156 | LGTSATNSVTYKKVENTVLPK | 2177 | PGTSATNSVTYKKVENTVLPK |
| | 2157 | SLGTSATNSVTYKKVENTVLP | 2178 | SPGTSATNSVTYKKVENTVLP |
| | 2158 | GSLGTSATNSVTYKKVENTVL | 2179 | GSPGTSATNSVTYKKVENTVL |
| | 2159 | VGSLGTSATNSVTYKKVENTV | 2180 | VGSPGTSATNSVTYKKVENTV |
| | 2160 | EVGSLGTSATNSVTYKKVENT | 2181 | EVGSPGTSATNSVTYKKVENT |
| | 2161 | REVGSLGTSATNSVTYKKVEN | 2182 | REVGSPGTSATNSVTYKKVEN |
| | 2162 | QREVGSLGTSATNSVTYKKVE | 2183 | QREVGSPGTSATNSVTYKKVE |
| | 2163 | DQREVGSLGTSATNSVTYKKV | 2184 | DQREVGSPGTSATNSVTYKKV |
| | 2164 | GDQREVGSLGTSATNSVTYKK | 2185 | GDQREVGSPGTSATNSVTYKK |
| | 2165 | TGDQREVGSLGTSATNSVTYK | 2186 | TGDQREVGSPGTSATNSVTYK |
| | 2166 | MTGDQREVGSLGTSATNSVTY | 2187 | MTGDQREVGSPGTSATNSVTY |
| | 2167 | EMTGDQREVGSLGTSATNSVT | 2188 | EMTGDQREVGSPGTSATNSVT |
| | 2168 | LEMTGDQREVGSLGTSATNSV | 2189 | LEMTGDQREVGSPGTSATNSV |
| | 2169 | TLEMTGDQREVGSLGTSATNS | 2190 | TLEMTGDQREVGSPGTSATNS |
| | 2170 | LTLEMTGDQREVGSLGTSATN | 2191 | LTLEMTGDQREVGSPGTSATN |
| | 2171 | ILTLEMTGDQREVGSLGTSAT | 2192 | ILTLEMTGDQREVGSPGTSAT |
| | 2172 | AILTLEMTGDQREVGSLGTSA | 2193 | AILTLEMTGDQREVGSPGTSA |
| | 2173 | LAILTLEMTGDQREVGSLGTS | 2194 | LAILTLEMTGDQREVGSPGTS |
| | 2174 | SLAILTLEMTGDQREVGSLGT | 2195 | SLAILTLEMTGDQREVGSPGT |
| | 2175 | LSLAILTLEMTGDQREVGSLG | 2196 | LSLAILTLEMTGDQREVGSPG |
| | 2176 | NLSLAILTLEMTGDQREVGSL | 2197 | NLSLAILTLEMTGDQREVGSP |

TABLE 97

| | ns-SNP TIPs | | | |
|---|---|---|---|---|
| | Major allele | | Minor allele | |
| | SEQ | | SEQ | |
| | ID | | ID | |
| F8 ns-SNPs | NO: | I1668 | NO: | V1668 |
| I1668V | 2198 | ISVEMKKEDFDIYDEDENQSP | 2219 | VSVEMKKEDFDIYDEDENQSP |
| | 2199 | TISVEMKKEDFDIYDEDENQS | 2220 | TVSVEMKKEDFDIYDEDENQS |
| | 2200 | DTISVEMKKEDFDIYDEDENQ | 2221 | DTVSVEMKKEDFDIYDEDENQ |
| | 2201 | DDTISVEMKKEDFDIYDEDEN | 2222 | DDTVSVEMKKEDFDIYDEDEN |
| | 2202 | YDDTISVEMKKEDFDIYDEDE | 2223 | YDDTVSVEMKKEDFDIYDEDE |
| | 2203 | DYDDTISVEMKKEDFDIYDED | 2224 | DYDDTVSVEMKKEDFDIYDED |
| | 2204 | IDYDDTISVEMKKEDFDIYDE | 2225 | IDYDDTVSVEMKKEDFDIYDE |
| | 2205 | EIDYDDTISVEMKKEDFDIYD | 2226 | EIDYDDTVSVEMKKEDFDIYD |
| | 2206 | EEIDYDDTISVEMKKEDFDIY | 2227 | EEIDYDDTVSVEMKKEDFDIY |
| | 2207 | QEEIDYDDTISVEMKKEDFDI | 2228 | QEEIDYDDTVSVEMKKEDFDI |
| | 2208 | DQEEIDYDDTISVEMKKEDFD | 2229 | DQEEIDYDDTVSVEMKKEDFD |
| | 2209 | SDQEEIDYDDTISVEMKKEDF | 2230 | SDQEEIDYDDTVSVEMKKEDF |
| | 2210 | QSDQEEIDYDDTISVEMKKED | 2231 | QSDQEEIDYDDTVSVEMKKED |
| | 2211 | LQSDQEEIDYDDTISVEMKKE | 2232 | LQSDQEEIDYDDTVSVEMKKE |
| | 2212 | TLQSDQEEIDYDDTISVEMKK | 2233 | TLQSDQEEIDYDDTVSVEMKK |
| | 2213 | TTLQSDQEEIDYDDTISVEMK | 2234 | TTLQSDQEEIDYDDTVSVEMK |
| | 2214 | RTTLQSDQEEIDYDDTISVEM | 2235 | RTTLQSDQEEIDYDDTVSVEM |
| | 2215 | TRTTLQSDQEEIDYDDTISVE | 2236 | TRTTLQSDQEEIDYDDTVSVE |
| | 2216 | ITRTTLQSDQEEIDYDDTISV | 2237 | ITRTTLQSDQEEIDYDDTVSV |
| | 2217 | EITRTTLQSDQEEIDYDDTIS | 2238 | EITRTTLQSDQEEIDYDDTVS |
| | 2218 | REITRTTLQSDQEEIDYDDTI | 2239 | REITRTTLQSDQEEIDYDDTV |

TABLE 98

| | ns-SNP TIPs | | | |
|---|---|---|---|---|
| | Major allele | | Minor allele | |
| | SEQ | | SEQ | |
| | ID | | ID | |
| F8 ns-SNPs | NO: | E2004 | NO: | K2004 |
| E2004K | 2240 | EHLHAGMSTLFLVYSNKCQTP | 2261 | KHLHAGMSTLFLVYSNKCQTP |
| | 2241 | GEHLHAGMSTLFLVYSNKCQT | 2262 | GKHLHAGMSTLFLVYSNKCQT |
| | 2242 | IGEHLHAGMSTLFLVYSNKCQ | 2263 | IGKHLHAGMSTLFLVYSNKCQ |
| | 2243 | LIGEHLHAGMSTLFLVYSNKC | 2264 | LIGKHLHAGMSTLFLVYSNKC |
| | 2244 | CLIGEHLHAGMSTLFLVYSNK | 2265 | CLIGKHLHAGMSTLFLVYSNK |
| | 2245 | ECLIGEHLHAGMSTLFLVYSN | 2266 | ECLIGKHLHAGMSTLFLVYSN |
| | 2246 | VECLIGEHLHAGMSTLFLVYS | 2267 | VECLIGKHLHAGMSTLFLVYS |
| | 2247 | RVECLIGEHLHAGMSTLFLVY | 2268 | RVECLIGKHLHAGMSTLFLVY |
| | 2248 | WRVECLIGEHLHAGMSTLFLV | 2269 | WRVECLIGKHLHAGMSTLFLV |
| | 2249 | IWRVECLIGEHLHAGMSTLFL | 2270 | IWRVECLIGKHLHAGMSTLFL |
| | 2250 | GIWRVECLIGEHLHAGMSTLF | 2271 | GIWRVECLIGKHLHAGMSTLF |
| | 2251 | AGIWRVECLIGEHLHAGMSTL | 2272 | AGIWRVECLIGKHLHAGMSTL |
| | 2252 | KAGIWRVECLIGEHLHAGMST | 2273 | KAGIWRVECLIGKHLHAGMST |
| | 2253 | SKAGIWRVECLIGEHLHAGMS | 2274 | SKAGIWRVECLIGKHLHAGMS |
| | 2254 | PSKAGIWRVECLIGEHLHAGM | 2275 | PSKAGIWRVECLIGKHLHAGM |
| | 2255 | LPSKAGIWRVECLIGEHLHAG | 2276 | LPSKAGIWRVECLIGKHLHAG |
| | 2256 | MLPSKAGIWRVECLIGEHLHA | 2277 | MLPSKAGIWRVECLIGKHLHA |
| | 2257 | EMLPSKAGIWRVECLIGEHLH | 2278 | EMLPSKAGIWRVECLIGKHLH |
| | 2258 | VEMLPSKAGIWRVECLIGEHL | 2279 | VEMLPSKAGIWRVECLIGKHL |
| | 2259 | TVEMLPSKAGIWRVECLIGEH | 2280 | TVEMLPSKAGIWRVECLIGKH |
| | 2260 | ETVEMLPSKAGIWRVECLIGE | 2281 | ETVEMLPSKAGIWRVECLIGK |

TABLE 99

| | ns-SNP TIPs | | | |
|---|---|---|---|---|
| | Major allele | | Minor allele | |
| | SEQ | | SEQ | |
| | ID | | ID | |
| F8 ns-SNPs | NO: | V2223 | NO: | M2223 |
| V2223M | 2282 | VNNPKEWLQVDFQKTMKVTGV | 2303 | MNNPKEWLQVDFQKTMKVTGV |
| | 2283 | QVNNPKEWLQVDFQKTMKVTG | 2304 | QMNNPKEWLQVDFQKTMKVTG |
| | 2284 | PQVNNPKEWLQVDFQKTMKVT | 2305 | PQMNNPKEWLQVDFQKTMKVT |
| | 2285 | RPQVNNPKEWLQVDFQKTMKV | 2306 | RPQMNNPKEWLQVDFQKTMKV |
| | 2286 | WRPQVNNPKEWLQVDFQKTMK | 2307 | WRPQMNNPKEWLQVDFQKTMK |
| | 2287 | AWRPQVNNPKEWLQVDFQKTM | 2308 | AWRPQMNNPKEWLQVDFQKTM |
| | 2288 | NAWRPQVNNPKEWLQVDFQKT | 2309 | NAWRPQMNNPKEWLQVDFQKT |
| | 2289 | SNAWRPQVNNPKEWLQVDFQK | 2310 | SNAWRPQMNNPKEWLQVDFQK |
| | 2290 | RSNAWRPQVNNPKEWLQVDFQ | 2311 | RSNAWRPQMNNPKEWLQVDFQ |
| | 2291 | GRSNAWRPQVNNPKEWLQVDF | 2312 | GRSNAWRPQMNNPKEWLQVDF |
| | 2292 | QGRSNAWRPQVNNPKEWLQVD | 2313 | QGRSNAWRPQMNNPKEWLQVD |
| | 2293 | LQGRSNAWRPQVNNPKEWLQV | 2314 | LQGRSNAWRPQMNNPKEWLQV |
| | 2294 | HLQGRSNAWRPQVNNPKEWLQ | 2315 | HLQGRSNAWRPQMNNPKEWLQ |
| | 2295 | LHLQGRSNAWRPQVNNPKEWL | 2316 | LHLQGRSNAWRPQMNNPKEWL |
| | 2296 | RLHLQGRSNAWRPQVNNPKEW | 2317 | RLHLQGRSNAWRPQMNNPKEW |
| | 2297 | ARLHLQGRSNAWRPQVNNPKE | 2318 | ARLHLQGRSNAWRPQMNNPKE |
| | 2298 | KARLHLQGRSNAWRPQVNNPK | 2319 | KARLHLQGRSNAWRPQMNNPK |
| | 2299 | SKARLHLQGRSNAWRPQVNNP | 2320 | SKARLHLQGRSNAWRPQMNNP |
| | 2300 | PSKARLHLQGRSNAWRPQVNN | 2321 | PSKARLHLQGRSNAWRPQMNN |
| | 2301 | SPSKARLHLQGRSNAWRPQVN | 2322 | SPSKARLHLQGRSNAWRPQMN |
| | 2302 | WSPSKARLHLQGRSNAWRPQV | 2323 | WSPSKARLHLQGRSNAWRPQM |

TABLE 100

| | ns-SNP TIPs | | | |
|---|---|---|---|---|
| | Major allele | | Minor allele | |
| | SEQ | | SEQ | |
| | ID | | ID | |
| F8 ns-SNPs | NO: | M2238V | NO: | M2238V |
| M2238V | 2324 | MKVTGVTTQGVKSLLTSMYVK | 2345 | VKVTGVTTQGVKSLLTSMYVK |
| | 2325 | TMKVTGVTTQGVKSLLTSMYV | 2346 | TVKVTGVTTQGVKSLLTSMYV |
| | 2326 | KTMKVTGVTTQGVKSLLTSMY | 2347 | KTVKVTGVTTQGVKSLLTSMY |
| | 2327 | QKTMKVTGVTTQGVKSLLTSM | 2348 | QKTVKVTGVTTQGVKSLLTSM |
| | 2328 | FQKTMKVTGVTTQGVKSLLTS | 2349 | FQKTVKVTGVTTQGVKSLLTS |
| | 2329 | DFQKTMKVTGVTTQGVKSLLT | 2350 | DFQKTVKVTGVTTQGVKSLLT |
| | 2330 | VDFQKTMKVTGVTTQGVKSLL | 2351 | VDFQKTVKVTGVTTQGVKSLL |
| | 2331 | QVDFQKTMKVTGVTTQGVKSL | 2352 | QVDFQKTVKVTGVTTQGVKSL |
| | 2332 | LQVDFQKTMKVTGVTTQGVKS | 2353 | LQVDFQKTVKVTGVTTQGVKS |
| | 2333 | WLQVDFQKTMKVTGVTTQGVK | 2354 | WLQVDFQKTVKVTGVTTQGVK |
| | 2334 | EWLQVDFQKTMKVTGVTTQGV | 2355 | EWLQVDFQKTVKVTGVTTQGV |
| | 2335 | KEWLQVDFQKTMKVTGVTTQG | 2356 | KEWLQVDFQKTVKVTGVTTQG |
| | 2336 | PKEWLQVDFQKTMKVTGVTTQ | 2357 | PKEWLQVDFQKTVKVTGVTTQ |
| | 2337 | NPKEWLQVDFQKTMKVTGVTT | 2358 | NPKEWLQVDFQKTVKVTGVTT |
| | 2338 | NNPKEWLQVDFQKTMKVTGVT | 2359 | NNPKEWLQVDFQKTVKVTGVT |
| | 2339 | VNNPKEWLQVDFQKTMKVTGV | 2360 | VNNPKEWLQVDFQKTVKVTGV |
| | 2340 | QVNNPKEWLQVDFQKTMKVTG | 2361 | QVNNPKEWLQVDFQKTVKVTG |
| | 2341 | PQVNNPKEWLQVDFQKTMKVT | 2362 | PQVNNPKEWLQVDFQKTVKVT |
| | 2342 | RPQVNNPKEWLQVDFQKTMKV | 2363 | RPQVNNPKEWLQVDFQKTVKV |
| | 2343 | WRPQVNNPKEWLQVDFQKTMK | 2364 | WRPQVNNPKEWLQVDFQKTV |
| | 2344 | AWRPQVNNPKEWLQVDFQKTM | 2365 | AWRPQVNNPKEWLQVDFQKTV |

TABLE 101

| | ns-SNP TIPs | | | |
|---|---|---|---|---|
| | Major allele | | Minor allele | |
| | SEQ | | SEQ | |
| | ID | | ID | |
| F8 ns-SNPs | NO: | P2292 | NO: | S2292 |
| P2292S | 2366 | PVVNSLDPPLLTRYLRIHPQS | 2387 | SVVNSLDPPLLTRYLRIHPQS |
| | 2367 | TPVVNSLDPPLLTRYLRIHPQ | 2388 | TSVVNSLDPPLLTRYLRIHPQ |
| | 2368 | FTPVVNSLDPPLLTRYLRIHP | 2389 | FTSVVNSLDPPLLTRYLRIHP |
| | 2369 | SFTPVVNSLDPPLLTRYLRIH | 2390 | SFTSVVNSLDPPLLTRYLRIH |
| | 2370 | DSFTPVVNSLDPPLLTRYLRI | 2391 | DSFTSVVNSLDPPLLTRYLRI |
| | 2371 | QDSFTPVVNSLDPPLLTRYLR | 2392 | QDSFTSVVNSLDPPLLTRYLR |
| | 2372 | NQDSFTPVVNSLDPPLLTRYL | 2393 | NQDSFTSVVNSLDPPLLTRYL |
| | 2373 | GNQDSFTPVVNSLDPPLLTRY | 2394 | GNQDSFTSVVNSLDPPLLTRY |
| | 2374 | QGNQDSFTPVVNSLDPPLLTR | 2395 | QGNQDSFTSVVNSLDPPLLTR |
| | 2375 | FQGNQDSFTPVVNSLDPPLLT | 2396 | FQGNQDSFTSVVNSLDPPLLT |
| | 2376 | VFQGNQDSFTPVVNSLDPPLL | 2397 | VFQGNQDSFTSVVNSLDPPLL |
| | 2377 | KVFQGNQDSFTPVVNSLDPPL | 2398 | KVFQGNQDSFTSVVNSLDPPL |
| | 2378 | VKVFQGNQDSFTPVVNSLDPP | 2399 | VKVFQGNQDSFTSVVNSLDPP |
| | 2379 | KVKVFQGNQDSFTPVVNSLDP | 2400 | KVKVFQGNQDSFTSVVNSLDP |
| | 2380 | GKVKVFQGNQDSFTPVVNSLD | 2401 | GKVKVFQGNQDSFTSVVNSLD |
| | 2381 | NGKVKVFQGNQDSFTPVVNSL | 2402 | NGKVKVFQGNQDSFTSVVNSL |
| | 2382 | QNGKVKVFQGNQDSFTPVVNS | 2403 | QNGKVKVFQGNQDSFTSVVNS |
| | 2383 | FQNGKVKVFQGNQDSFTPVVN | 2404 | FQNGKVKVFQGNQDSFTSVVN |
| | 2384 | FFQNGKVKVFQGNQDSFTPVV | 2405 | FFQNGKVKVFQGNQDSFTSVV |
| | 2385 | LFFQNGKVKVFQGNQDSFTPV | 2406 | LFFQNGKVKVFQGNQDSFTSV |
| | 2386 | TLFFQNGKVKVFQGNQDSFTP | 2407 | TLFFQNGKVKVFQGNQDSFTS |

In certain aspects of the present invention, compositions directed to specific TIPs and TIP sets described in Table 102, and methods using the compositions thereof, are provided herein. In one embodiment, at least one or more TIPs comprising at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, or at least 20 amino acids, including at the reference locus based on an intron 22 inversion, identified in Table 102 are provided. In one embodiment, at least one TIP set comprising at least 9 peptides, at least 10 peptides, at least 11 peptides, at least 12 peptides, at least 13 peptides, at 14 peptides, 15 peptides, at least 16 peptides, at least 17 peptides, at least 18 peptides, at least 19 peptides, or at least 20 peptides, wherein the first peptide of the set comprises a first reference locus M from the reference locus MV at its first amino acid position, the second peptide of the set comprises the reference locus M at its second amino acid position, and each successive peptide in the set comprises the reference locus M at an amino acid position frame-shifted one position downstream from the reference locus position of the preceding peptide, and wherein the last peptide of the set has the reference locus V in its last amino acid position, wherein the TIP sets are generated from the TIPs identified in Table 102, are provided herein (reference locus underlined and bolded). Table 102 is provided below.

TABLE 102

| FVIIIrp Reference | SEQ ID | |
|---|---|---|
| Locus | NO: | F8_{I22I} TIPs |
| MV | 2408 | MVFFGNVDSSGIKHNIFNPPI |
| | 2409 | LMVFGNVDSSGIKHNIFNPP |
| | 2410 | TLMVFFGNVDSSGIKHNIFNP |
| | 2411 | GTLMVFFGNVDSSGIKHNIFN |
| | 2412 | TGTLMVFFGNVDSSGIKHNIF |
| | 2413 | STGTLMVFFGNVDSSGIKHNI |
| | 2414 | NSTGTLMVFFGNVDSSGIKHN |
| | 2415 | GNSTGTLMVFFGNVDSSGIKH |
| | 2416 | RGNSTGTLMVFFGNVDSSGIK |
| | 2417 | YRGNSTGTLMVFFGNVDSSGI |
| | 2418 | TYRGNSTGTLMVFFGNVDSSG |
| | 2419 | QTYRGNSTGTLMVFFGNVDSS |
| | 2420 | WQTYRGNSTGTLMVFFGNVDS |
| | 2421 | KWQTYRGNSTGTLMVFFGNVD |
| | 2422 | KKWQTYRGNSTGTLMVFFGNV |
| | 2423 | GKKWQTYRGNSTGTLMVFFGN |
| | 2424 | DGKKWQTYRGNSTGTLMVFFG |
| | 2425 | LDGKKWQTYRGNSTGTLMVFF |
| | 2426 | SLDGKKWQTYRGNSTGTLMVF |
| | 2427 | YSLDGKKWQTYRGNSTGTLMV |

In certain aspects of the present invention, compositions directed to specific TIPs and TIP sets described in Table 103, and methods using the compositions thereof, are provided herein. In one embodiment, at least one or more TIPs comprising at least 9 amino acids, at least 10 amino acids, at least 11 amino acids, at least 12 amino acids, at least 13 amino acids, at least 14 amino acids, or at least 15 amino acids, including at the reference locus based on the use of a BDD-rFVIIIrp containing a synthetic linker, identified in Table 103 are provided. In one embodiment, at least one TIP set comprising at least 5 peptides, at least 6 peptides, at least 7 peptides, at least 8 peptides, at least 9 peptides, at least 10 peptides, or at least 11 peptides, wherein the first peptide of the set comprises an amino acid residue located +1 residues upstream from the reference locus at its first amino acid position and the reference locus is positioned as the second amino acid, the second peptide of the set comprises a reference locus at its third amino acid position, and each successive peptide in the set comprises a reference locus at an amino acid position frame-shifted one position downstream from the reference locus position of the preceding peptide, and wherein the last peptide of the set has the reference locus in its fourth from the last amino acid position, wherein the TIP sets are generated from the TIPs identified in Table 103, are provided herein (reference locus bolded and underlined). Tables 103 are provided below.

TABLE 103

| Reference | BDD- | SEQ | |
|---|---|---|---|
| Locus Position | rFVIIIrp | ID | |
| within BDD-rFVIIIrp | Linker | NO: | TIP Set |
| 743 | S-Q-N | 2428 | FSQNPPVLKRHQREI |
| | | 2429 | SFSQNPPVLKRHQRE |
| | | 2430 | RSFSQNPPVLKRHQR |
| | | 2431 | PRSFSQNPPVLKRHQ |
| | | 2432 | EPRSFSQNPPVLKRH |
| | | 2433 | IEPRSFSQNPPVLKR |
| | | 2434 | AIEPRSFSQNPPVLK |
| | | 2435 | NAIEPRSFSQNPPVL |
| | | 2436 | NNAIEPRSFSQNPPV |
| | | 2437 | KNNAIEPRSFSQNPP |
| | | 2438 | SKNNAIEPRSFSQNP |

The TIPs and TIP sets described herein are synthesized using any known peptide synthesizing protocol. For example, peptides of the present invention can be synthesized by a 9-fluorenylmethoxy-carbonyl (Fmoc) method on an automated peptide synthesizer, for example an automated Rainen Symphony/Protein Technologies synthesizer. Peptides can be purified by HPLC to remove impurities.

Association with Carrier

The TIPs described herein can be associated with a carrier. Accordingly, compositions and methods using such compositions thereof are contemplated herein comprising TIPs as described herein in association with a carrier.

Carrier can include for example, natural or synthetic compounds. In some embodiments, a carrier includes cell-based particles, including cells such as antigen presenting cells including dendritic cells such as immature dendritic cells. In certain embodiments, the carrier can be, but are not limited to, a B cells, T cell, a leukocyte such as a splenic leukocytes or isologous leukocyte. The TIP can be bound to the cells, or alternatively, ingested by or pulsed into the cells for processing and subsequent presentation.

In one embodiment the TIPs are coupled to isologous splenocytes using ECDI as described in Getts et al. (Micro-particles bearing encephalitogenic peptides induce T-cell tolerance and ameliorate experimental autoimmune encephalomyelitis. Nature Biotechnology 2012 (http://www.nature.com/doifinder/10.1038/nbt.2434).

In some embodiments, the carrier is a hapten or immunoglobulin including but not limited to a fragmented IgG Fc fragment. In one embodiment, the carrier is a haptenated immunoglobulin.

In one embodiment, the carrier molecule is mannose-6-phosphate.

In some embodiments, the carrier is a micro- or nano-particle, such as a polymeric micro- or nano-particle. Micro- or nano-particles may comprise natural polymers, including but not limited to chitosan, alginate, dextran, gelatin, and albumin, and synthetic polymers such as, but not limited to, poly(lactide-co-glycolide) (PLGA), (3-hydroxybutyrate-co-3-hydroxyvalerate) (PHBV), poly(sebacic anhydride), poly(ε-caprolactone), polystyrene, thermoresponsive (i.e., NIPAAm and CMCTS-g-PDEA) and pH-responsive (i.e., Eudragit L100, Eudragit S and AQOAT AS-MG) polymers.

In one embodiment, the polymeric micro- or nano-particle is between about 0.1 nm to about 10000 nm, between about 1 nm to about 1000 nm, between about 10 nm and 1000 nm, between about 100 nm and 800 nm, between about 400 nm and 600 nm, or about 500 nm. In one embodiment, the micro- or nano-particles are about 0.1 nm, 0.5 nm, 1.0 nm, 5.0 nm, 10 nm, 25 nm, 50 nm, 75 nm, 100 nm, 150 nm, 200 nm, 250 nm, 300 nm, 400 nm, 450 nm, 500 nm, 550 nm, 600 nm, 650 nm, 700 nm, 750 nm, 800 nm, 850 nm, 900 nm, 950 nm, 1000 nm, 1250 nm, 1500 nm, 1750 nm, or 2000 nm. In a particular embodiment, the TIPs are covalently coupled to a polystyrene particle, PLGA particle, PLGA-PEMA particle, PLA particle, or other micro- or nano-particle using an ECDI linker as described in Getts et al. (Microparticles bearing encephalitogenic peptides induce T-cell tolerance and ameliorate experimental autoimmune encephalomyelitis. Nature Biotechnology 2012 (http://www.nature.com/doifinder/10.1038/nbt.2434).

In a more particular embodiment, the carrier is a PLGA, PLGA-PEMA, PLA, or carboxylated polystyrene bead of from about 1 nm to about 5000 nm, from about 10 nm to about 2000 nm, from about 100 nm to about 1000 nm, more particularly from about 400 nm to about 600 nm, and even more particularly about 500 nm. TIPs are coupled to micro- or nano-particles, for example, as follows: 12.5 mg of micro- or nano-particles and 500 ug of peptide in the presence of 10 mg/ml ECDI.

In one embodiment, the carrier is a PLGA particle modified with PEMA (poly[ethylene-co-maleic acid]) as a surfactant to form a PLGA-PEMA particle, in diameter of from 1 nm to about 5000 nm, from about 10 nm to about 2000 nm, from about 100 nm to about 1000 nm, more particularly from about 400 nm to about 600 nm, and even more particularly about 500 nm. Methods for production of PLGA-PEMA and for conjugation of PLGA-PEMA to peptides exist in the art (Hunter, Z. et al. A Biodegradable Nanoparticle Platform for the Induction of Antigen-Specific Immune Tolerance for Treatment of Autoimmune Disease. ACS Nano 140227095031005 (2014). doi:10.1021/nn405033r).

In some embodiments, the carrier can be solid or hollow and can comprise one or more layers. In some embodiments, each layer has a unique composition and unique properties relative to the other layer(s). To give but one example, the carrier may have a core/shell structure, wherein the core is one layer (e.g., a polymeric core) and the shell is a second layer (e.g., a lipid bilayer or monolayer). In some embodiments, the carrier may comprise a plurality of different layers. In some embodiments, the TIPs are incorporated into or surrounded by one or more layers.

In some embodiments, carriers may optionally comprise one or more lipids. In some embodiments, a carrier may comprise a liposome. In some embodiments, a carrier may comprise a lipid bilayer. In some embodiments, a carrier may comprise a lipid monolayer. In some embodiments, a carrier may comprise a micelle. In some embodiments, a carrier may comprise a core comprising a polymeric matrix surrounded by a lipid layer (e.g., lipid bilayer, lipid monolayer, etc.). In some embodiments, a carrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, carriers may comprise metal particles, quantum dots, ceramic particles, etc. In some embodiments, a non-polymeric carrier is an aggregate of non-polymeric components, such as an aggregate of metal atoms (e.g., gold atoms).

In some embodiments, carriers may optionally comprise one or more amphiphilic entities. In some embodiments, an amphiphilic entity can promote the production of carriers with increased stability, improved uniformity, or increased viscosity. In some embodiments, amphiphilic entities are associated with the interior surface of a lipid membrane (e.g., lipid bilayer, lipid monolayer, etc.). Many amphiphilic entities known in the art are suitable for use in making carriers useful in the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of carriers to be used in accordance with the present invention.

In some embodiments, a carrier may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In some embodiments, the carrier does not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, the associated carrier can comprise one or more polymers. In some embodiments, the carrier comprises one or more polymers that are a non-methoxy-terminated, pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the carriers are non-methoxy-terminated, pluronic polymers. In some embodiments, all of the polymers that make up the carrier are non-methoxy-terminated, pluronic polymers. In some embodiments, the carrier comprises one or more polymers that are a non-methoxy-terminated polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the carriers are non-methoxy-terminated polymers. In some embodiments, all of the polymers that make up the carrier are non-methoxy-terminated polymers. In some embodiments, the carrier comprises one or more polymers that do not comprise pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the carrier do not comprise pluronic polymer. In some embodiments, all of the polymers that make up the carrier do not comprise pluronic polymer. In some embodiments, such a polymer are surrounded by a coating layer (e.g., liposome, lipid monolayer, micelle, etc.). In some embodiments, various elements of the carrier are coupled with the polymer.

Other examples of polymers include, but are not limited to polyethylenes, polycarbonates (e.g., poly(1,3-dioxan-2one)), polyanhydrides (e.g., poly(sebacic anhydride)), polypropylfumerates, polyamides (e.g., polycaprolactam), polyacetals, polyethers, polyesters (e.g., polylactide, polyglycolide, polylactide-co-glycolide, polycaprolactone, polyhydroxyacid (e.g., poly((β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, carriers include polymers which have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers are hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a carrier comprising a hydrophilic polymeric matrix generates a hydrophilic environment within the carrier. In some embodiments, polymers are hydrophobic. In some embodiments, a carrier comprising a hydrophobic polymeric matrix generates a hydrophobic environment within the carrier. Selection of the hydrophilicity or hydrophobicity of the polymer may have an impact on the nature of materials that are incorporated (e.g., coupled) within the carrier.

In some embodiments, polymers may be modified with one or more moieties and/or functional groups. A variety of moieties or functional groups are used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers are cationic polymers. In general, cationic polymers are able to condense and/or protect negatively charged strands of nucleic acids (e.g., DNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines. In embodiments, the inventive carriers may not comprise (or may exclude) cationic polymers.

In some embodiments, polymers are degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

Polymers are linear or branched polymers. In some embodiments, polymers are dendrimers. In some embodiments, polymers are substantially cross-linked to one another. In some embodiments, polymers are substantially free of cross-links. In some embodiments, polymers are used in accordance with the present invention without undergoing a cross-linking step. It is further to be understood that a carrier may comprise block copolymers, graft copolymers, blends, mixtures, and/or adducts of any of the foregoing and other polymers. Those skilled in the art will recognize that the polymers listed herein represent an exemplary, not comprehensive, list of polymers that are of use in accordance with the present invention.

The TIPs of the present invention are coupled to the carrier by any of a number of methods. For example, the coupling can be a result of bonding between the TIPs and the carrier. This bonding can result in the TIP being attached to the surface of the carrier and/or contained within (encapsulated) the carrier. In some embodiments, however, the TIPs are encapsulated by the carrier as a result of the structure of the carrier rather than bonding to the carrier. In some embodiments, the carrier comprises a polymer as provided herein, and the TIPs are coupled to the carrier.

When coupling occurs as a result of bonding between the TIP and carrier, the coupling may occur via a coupling moiety. A coupling moiety can be any moiety through which TIP is bonded to a carrier. Such moieties include covalent bonds, such as an amide bond or ester bond, as well as separate molecules that bond (covalently or non-covalently) the TIP to the carrier. Such molecules include linkers or polymers or a unit thereof. For example, the coupling moiety can comprise a charged polymer to which TIP electrostatically binds. As another example, the coupling moiety can comprise a polymer or unit thereof to which it is covalently bonded.

In a particular embodiment, the TIP is coupled to the carrier using an ethylene carbodiimide (ECDI) moiety. ECDI is commercially available and TIPs are linked thereto as described, for example, in Getts et al. Microparticles bearing encephalitogenic peptides induce T-cell tolerance and ameliorate experimental autoimmune encephalomyelitis. Nature Biotechnology 2012 (http://www.nature.com/doifinder/10.1038/nbt.2434).

In certain embodiments, the coupling of the TIP to the carrier are through a covalent linker. In embodiments, TIPs are covalently coupled to the external surface via a 1,2,3-triazole linker formed by the 1,3-dipolar cycloaddition reaction of azido groups on the surface of the carrier. Such cycloaddition reactions are for example performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, the covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component with the carboxylic acid group of a second component such as the carrier. The amide bond in the linker are made using any of the conventional amide bond forming reactions with suitably protected amino acids and activated carboxylic acid such N-hydroxysuccinimide-activated ester. A disulfide linker is made via the formation of a disulfide (S—S) bond between two sulfur atoms of the form, for instance, of R1-S—S—R2. A disulfide bond are formed by thiol exchange of a component containing thiol/mercaptan group (—SH) with another activated thiol group on a polymer or carrier or a carrier containing thiol/mercaptan groups with a component containing activated thiol group.

In some embodiments, a polymer containing an azide or alkyne group, terminal to the polymer chain is prepared. This polymer is then used to prepare a carrier in such a manner that a plurality of the alkyne or azide groups are positioned on the surface of that carrier. Alternatively, the carrier are prepared by another route, and subsequently functionalized with alkyne or azide groups. The TIPs are prepared with the presence of either an alkyne (if the polymer contains an azide) or an azide (if the polymer contains an alkyne) group. The TIP is then allowed to react with the carrier via the 1,3-dipolar cycloaddition reaction with or without a catalyst which covalently couples the component to the particle through the 1,4-disubstituted 1,2,3-triazole linker.

A thioether linker is made by the formation of a sulfur-carbon (thioether) bond in the form, for instance, of R1-S—R2. Thioether are made by either alkylation of a thiol/mercaptan (—SH) group on one component with an alkylating group such as halide or epoxide on a second component. Thioether linkers can also be formed by Michael addition of a thiol/mercaptan group on one component to an electron-deficient alkene group on a second component containing a maleimide group or vinyl sulfone group as the Michael acceptor. In another way, thioether linkers are prepared by the radical thiol-ene reaction of thiol/mercaptan group on one component with an alkene group on a second component.

A hydrazone linker is made by the reaction of a hydrazide group on one component with an aldehyde/ketone group on the second component.

A hydrazide linker is formed by the reaction of a hydrazine group on one component with a carboxylic acid group on the second component. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component with an aldehyde or ketone group on the second component.

An urea or thiourea linker is prepared by the reaction of an amine group on one component with an isocyanate or thioisocyanate group on the second component.

An amidine linker is prepared by the reaction of an amine group on one component with an imidoester group on the second component.

An amine linker is made by the alkylation reaction of an amine group on one component with an alkylating group such as halide, epoxide, or sulfonate ester group on the second component. Alternatively, an amine linker can also be made by reductive amination of an amine group on one component with an aldehyde or ketone group on the second component with a suitable reducing reagent such as sodium cyanoborohydride or sodium triacetoxyborohydride.

A sulfonamide linker is made by the reaction of an amine group on one component with a sulfonyl halide (such as sulfonyl chloride) group on the second component.

A sulfone linker is made by Michael addition of a nucleophile to a vinyl sulfone. Either the vinyl sulfone or the nucleophile may be on the surface of the nanocarrier or attached to a component.

The TIP can also be conjugated to the carrier via non-covalent conjugation methods. For example, a negative charged TIP are conjugated to a positive charged carrier through electrostatic adsorption.

In embodiments, the TIP are attached to a polymer, for example polylactic acid-block-polyethylene glycol, prior to the assembly of the carrier or the carrier are formed with reactive or activatable groups on its surface. In the latter case, the TIP may be prepared with a group which is compatible with the attachment chemistry that is presented by the carriers' surface. In other embodiments, a TIP are attached to VLPs or liposomes using a suitable linker. A linker is a compound or reagent that capable of coupling two molecules together. In an embodiment, the linker are a homobifunctional or heterobifunctional reagent as described in Hermanson 2008. For example, a VLP or liposome carrier containing a carboxylic group on the surface are treated with a homobifunctional linker, adipic dihydrazide (ADH), in the presence of EDC to form the corresponding carrier with the ADH linker. The resulting ADH linked carrier is then conjugated with a TIP containing an acid group via the other end of the ADH linker on NC to produce the corresponding VLP or liposome TIP conjugate.

For detailed descriptions of available conjugation methods, see Hermanson G T “Bioconjugate Techniques”, 2nd Edition Published by Academic Press, Inc., 2008. In addition to covalent attachment the component are coupled by adsorption to a pre-formed carrier or it is coupled by encapsulation during the formation of the carrier.

Carriers may be prepared using a wide variety of methods known in the art. For example, carriers are formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

TIPs may be encapsulated into carriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into carriers may be used, including without limitation methods disclosed in U.S. Pat. No. 6,632,671 to Unger Oct. 14, 2003.

In certain embodiments, carriers are prepared by a nanoprecipitation process or spray drying. Conditions used in preparing carriers may be altered to yield particles of a desired size or property (e.g., hydrophobicity, hydrophilicity, external morphology, “stickiness,” shape, etc.). The method of preparing the carriers and the conditions (e.g., solvent, temperature, concentration, air flow rate, etc.) used may depend on the materials to be coupled to the carriers and/or the composition of the polymer matrix. If particles prepared by any of the above methods have a size range outside of the desired range, particles can be sized, for example, using a sieve.

TIPs can be associated with a cocktail of immune suppressants, including but not limited to, rapamycin and IL10.

Formulations

Compositions according to the invention may further comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, TIPs are suspended in sterile saline solution for injection together with a preservative.

The TIP compositions described herein can further comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

It is to be understood that the compositions of the invention are made in any suitable manner, and the invention is in no way limited to compositions that are produced using the methods described herein. Selection of an appropriate method may require attention to the properties of the particular moieties being associated.

In some embodiments, TIPs are manufactured under sterile conditions or are terminally sterilized. This can ensure that resulting compositions are sterile and non-infectious, thus improving safety when compared to non-sterile compositions. In some embodiments, TIPs may be lyophilized and stored in suspension or as lyophilized powder depending on the formulation strategy for extended periods without losing activity.

In certain embodiments, the TIPs described herein are associated with a carrier, for example coupled to a micro- or nano-particle. In certain embodiments, the amount of TIP (“load”) coupled to a carrier is based on the total weight of materials (weight/weight). Generally, the load is calculated as an average across a population of carriers, for example, microparticles. In one embodiment, the load of the TIPs on average across the population of carriers is between 0.0001% and 50%. In yet another embodiment, the load of the TIPs is between 0.01% and 20%. In a further embodiment, the load of the TIPs is between 0.1% and 10%. In still a further embodiment, the load of the TIPs is between 1% and 10%. In yet another embodiment, the load of the TIPs is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19% or at least 20% on average across a population of carriers. In yet a further embodiment, the load of the TIPs is 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20% on average across a population of carriers. In some embodiments of the above embodiments, the load of the TIPs is no more than 25% on average across a population of carriers.

In general, doses of the TIP are administered based on the total TIP contained in the composition. For example, doses of TIPs can range from about 10 μg/kg to about 100,000 μg/kg. from about 20 μg/kg to about 1000 μg/kg, from about 50 μg/kg to about 500 μg/kg, from about 75 μg/kg to about 250 μg/kg. In some embodiments, the total dose of TIPs for administration are at least about 5 μg, 10 μg, 15 μg, 20 μg, 25 μg, 35 μg, 40 μg, 50 μg, 60 μg, 75 μg, 80 μg, 90 μg, 100 μg, 125 μg, 150 μg, 200 μg, 250 μg, 300 μg, 350 μg, 400 μg, 500 μg or more. In some embodiments, the doses can range from about 0.1 mg/kg to about 100 mg/kg. In still other embodiments, the doses can range from about 0.1 mg/kg to about 25 mg/kg, about 25 mg/kg to about 50 mg/kg, about 50 mg/kg to about 75 mg/kg or about 75 mg/kg to about 100 mg/kg. Alternatively, the dose is administered based on the number of carrier micro- or nano-particles that provide the desired amount of TIPs. For example, useful doses include greater than 10⁶, 10⁷, 10⁸, 10⁹ or 10¹⁰ micro- or nano-particles per dose. Other examples of useful doses include from about 1×10⁶ to about 1×10¹⁰, about 1×10⁷ to about 1×10⁹ or about 1×10⁸ to about 1×10⁹ micro- or nano-particle carriers per dose.

In one embodiment, a single dose of TIPs for administration includes at least about 15 μg of peptide.

In one embodiment, the TIPs are associated, for example bound, with a cell, for example, including but not limited to, a splenic leukocyte. In general the total dose of TIPs bound to the cell for administration is at least about 5 μg, 10 μg, 15 μg, 20 μg, 25 μg, 35 μg, 40 μg, 50 μg, 60 μg, 75 μg, 80 μg, 90 μg, 100 μg, 125 μg, 150 μg, 200 μg, 250 μg, 300 μg, 350 μg, 400 μg, 500 μg or more. Alternatively, useful doses include from about 1×10⁶ to about 1×10¹⁰, about 1×10⁷ to about 1×10⁹ or about 1×10⁸ to about 1×10⁹ cells comprising bound TIP-peptide per dose.

Induction of Immunologic Tolerance

The TIP compositions is administered to the subject through any suitable approach. The amount and timing of administration can, of course, be dependent on the subject being treated, on the sFVIII deficiency, on the presence or absence of FVIIIrp inhibitors, the FVIIIrp to which the subject will be, is, or has received and the difference between amino acid sequences in the sFVIII and FVIIIrp, on the time course of the FVIIIrp treatment, on the manner of administration, and on the judgment of the prescribing physician. Thus, because of subject to subject variability, the dosages given below are a guideline and the physician can titrate doses of the TIP compositions to achieve the tolerance that the physician considers appropriate for the subject. In considering the degree of treatment desired, the physician can balance a variety of factors such as age and weight of the subject, presence of inhibitors, as well as presence of other diseases. Pharmaceutical formulations is prepared for any desired route of administration including, but not limited to, oral, intravenous, or aerosol administration, as discussed in greater detail below.

The TIPs of the current invention are administered to a subject in order to induce a tolerogenic immune response—that is an immune response that can lead to immune suppression specific to a specific rFVIIIrp antigen or immunogenic epitope. Such a tolerogenic immune response may include any reduction, delay, or inhibition in an undesired immune response specific to the rFVIIIrp antigen or epitope. Tolerogenic immune responses, therefore, can include the prevention of or reduction in inhibitors to a specific rFVIIIrp. Tolerogenic immune responses as provided herein include immunological tolerance. The tolerogenic immune response is the result of MHC Class II-restricted presentation and/or B cell presentation, or any other presentation leading to the minimized or reduced immunicity of the rFVIIIrp.

Tolerogenic immune responses may include a reduction in FVIIIrp antigen-specific antibody (inhibitor) production. The administration of the TIPs and peptide sets described herein may result in a reduction of measurable Bethesda titer units to a FVIIIrp in a subject that already has inhibitors to a FVIIIrp. Tolerogenic immune responses also include any response that leads to the stimulation, production, or recruitment of CD4+ Treg cells and/or CD8+ Treg cells. CD4+ Treg cells can express the transcription factor FoxP3 and inhibit inflammatory responses and autoimmune inflammatory diseases (Human regulatory T cells in autoimmune diseases. Cvetanovich G L, Hafler D A. Curr Opin Immunol. 2010 December; 22(6):753-60. Regulatory T cells and autoimmunity. Vila J, Isaacs J D, Anderson A E. Curr Opin Hematol. 2009 July; 16(4):274-9). Such cells also suppress T-cell help to B-cells and induce tolerance to both self and foreign antigens (Therapeutic approaches to allergy and autoimmunity based on FoxP3+ regulatory T-cell activation and expansion. Miyara M, Wing K, Sakaguchi S. J Allergy Clin Immunol. 2009 April; 123(4):749-55). CD4+ Treg cells recognize antigen when presented by Class II proteins on APCs. CD8+ Treg cells, which recognize antigens presented by Class I (and Qa-1), can also suppress T-cell help to B-cells and result in activation of antigen-specific suppression inducing tolerance to both self and foreign antigens. Disruption of the interaction of Qa-1 with CD8+ Treg cells has been shown to dysregulate immune responses and results in the development of auto-antibody formation and an autoimmune lethal systemic-lupus-erythematosus (Kim et al., Nature. 2010 Sep. 16, 467 (7313): 328-32). CD8+ Treg cells have also been shown to inhibit models of autoimmune inflammatory diseases including rheumatoid arthritis and colitis (CD4+CD25+ regulatory T cells in autoimmune arthritis. Oh S, Rankin A L, Caton A J. Immunol. Rev. 2010 January; 233(1):97-111. Regulatory T cells in inflammatory bowel disease. Boden E K, Snapper S B. Curr Opin Gastroenterol. 2008 November; 24(6):733-41). In some embodiments, the TIP compositions provided can effectively result in both types of responses (CD4+ Treg and CD8+Treg). In other embodiments, FoxP3 is induced in other immune cells, such as macrophages, iNKT cells, etc., and the compositions provided herein can result in one or more of these responses as well.

Tolerogenic immune responses also include, but are not limited to, the induction of regulatory cytokines, such as Treg cytokines; induction of inhibitory cytokines; the inhibition of inflammatory cytokines (e.g., IL-4, IL-1, IL-5, TNF-α, IL-6, GM-CSF, IFN-γ, IL-2, IL-9, IL-12, IL-17, IL-18, IL-21, IL-22, IL-23, M-CSF, C reactive protein, acute phase protein, chemokines (e.g., MCP-1, RANTES, MIP-1α, MIP-1β, MIG, ITAC or IP-10), the production of anti-inflammatory cytokines (e.g., IL-4, IL-13, IL-10, etc.), chemokines (e.g., CCL-2, CXCL8), proteases (e.g., MMP-3, MMP-9), leukotrienes (e.g., CysLT-1, CysLT-2), prostaglandins (e.g., PGE2) or histamines; the inhibition of polarization to a Th17, Th1, or Th2 immune response; the inhibition of effector cell-specific cytokines: Th17 (e.g., IL-17, IL-25), Th1 (IFN-γ), Th2 (e.g., IL-4, IL-13); the inhibition of Th1-, Th2- or TH17-specific transcription factors; the inhibition of proliferation of effector T cells; the induction of apoptosis of effector T cells; the induction of tolerogenic dendritic cell-specific genes, the induction of FoxP3 expression, the inhibition of IgE induction or IgE-mediated immune responses; the inhibition of antibody responses (e.g., antigen-specific antibody production); the inhibition of T helper cell response; the production of TGF-β and/or IL-10; the inhibition of effector function of autoantibodies (e.g., inhibition in the depletion of cells, cell or tissue damage or complement activation); etc.

Any of the foregoing may be measured in vivo in one or more animal models or may be measured in vitro. One of ordinary skill in the art is familiar with such in vivo or in vitro measurements. Tolerogenic immune responses are monitored using, for example, methods of assessing immune cell number and/or function, tetramer analysis, ELISPOT, flow cytometry-based analysis of cytokine expression, cytokine secretion, cytokine expression profiling, gene expression profiling, protein expression profiling, analysis of cell surface markers, PCR-based detection of immune cell receptor gene usage (see T. Clay et al., “Assays for Monitoring Cellular Immune Response to Active Immunotherapy of Cancer” Clinical Cancer Research 7:1127-1135 (2001)), etc. Tolerogenic immune responses may also be monitored using, for example, methods of assessing protein levels in plasma or serum, immune cell proliferation and/or functional assays, etc. In some embodiments, tolerogenic immune responses are monitored by assessing the induction of FoxP3.

In some embodiments, the reduction of an undesired immune response or generation of a tolerogenic immune response may be assessed by determining clinical endpoints, clinical efficacy, clinical symptoms, disease biomarkers and/or clinical scores. Tolerogenic immune responses can also be assessed with diagnostic tests to assess the presence or absence of inhibitors.

In one embodiment, administration of an effective amount of TIPs may result in the prevention, reduction, or elimination of inhibitors to a FVIIIrp, and in particular a rFVIIIrp. The presence of inhibitors are assessed by determining one or more antibody titers to the FVIIIrp using techniques known in the art and include Enzyme-linked Immunosorbent Assay (ELISA), inhibition liquid phase absorption assays (ILPAAs), rocket immunoelectrophoresis (RIE) assays, and line immunoelectrophoresis (LIE) assays.

The TIP compositions of the invention are administered in effective amounts, such as the effective amounts described elsewhere herein. Doses of dosage forms contain varying amounts of TIPs or TIP sets, according to the invention. The amount of TIPs present in the inventive dosage forms are varied according to the nature and number of the TIP, the therapeutic benefit to be accomplished, and other such parameters. In embodiments, dose ranging studies are conducted to establish optimal therapeutic amount of TIPs to be present in the dosage form. In embodiments, the TIPs are present in the dosage form in an amount effective to generate a tolerogenic immune response to a FVIIIrp epitope upon administration to a subject. It may be possible to determine amounts of the TIPs effective to generate a tolerogenic immune response using conventional dose ranging studies and techniques in subjects. Dosage forms may be administered at a variety of frequencies. In one embodiment, at least one administration of the dosage form is sufficient to generate a pharmacologically relevant response. In one embodiment, at least two administrations, at least three administrations, or at least four administrations or more, of the dosage form are utilized to ensure a pharmacologically relevant response.

Prophylactic administration of the TIP compositions described herein is initiated prior to the onset of inhibitor development, or therapeutic administration is initiated after inhibitor development is established.

In some embodiments, administration of TIPs is undertaken e.g., prior to administration of the rFVIIIrp. In exemplary embodiments, TIPs are administered at one or more times including, but not limited to, 30, 25, 20, 15, 14, 13, 12, 11, 10, 9, 8, 7, 6, 5, 4, 3, 2, 1, or 0 days prior to administration of the rFVIIIrp. In addition or alternatively, TIPs are administered to a subject following administration of the rFVIIIrp. In exemplary embodiments, TIPs are administered at one or more times including, but not limited to, 1, 2, 3, 4, 5, 6, 7, 8, 9, 10, 11, 12, 13, 14, 15, 20, 25, 30, etc. days following administration of rFVIIIrp.

In some embodiments, a maintenance dose is administered to a subject after an TIP initial administration has resulted in a tolerogenic response in the subject, for example to maintain the tolerogenic effect achieved after the initial dose, to prevent an undesired immune reaction in the subject, or to prevent the subject becoming a subject at risk of experiencing an undesired immune response or an undesired level of an immune response. In some embodiments, the maintenance dose is the same dose as the initial dose the subject received. In some embodiments, the maintenance dose is a lower dose than the initial dose. For example, in some embodiments, the maintenance dose is about ¾, about ⅔, about ½, about ⅓, about ¼, about ⅛, about 1/10, about 1/20, about 1/25, about 1/50, about 1/100, about 1/1,000, about 1/10,000, about 1/100,000, or about 1/1,000,000 (weight/weight) of the initial dose.

In some aspects, methods and compositions provided herein are useful in conjunction with established means of ITI against FVIII. ITI protocols for hemophilia patients, including patients with high titer inhibitors against FVIII, are known in the art and are generally described, e.g., in Mariani et al., Thromb Haemost., 72: 155-158 (1994) and DiMichele et al., Thromb Haemost. Suppl 130 (1999). Administration of TIP composition described herein are conducted before, after, and/or concurrently with established ITI protocols and/or variations thereof. For example, in some aspects, methods provide herein increase the effectiveness of established ITI protocols (e.g., the degree and/or likelihood of successful treatment) and/or reduce associated costs or side effects. In further aspects, methods provide herein allow established ITI protocols to be beneficially modified, e.g., to decrease the frequency, duration, and/or dose of FVIII administration.

The compositions of the invention are administered by a variety of routes, including but not limited to subcutaneous, intranasal, oral, intravenous, intraperitoneal, intramuscular, transmucosal, transmucosal, sublingual, rectal, ophthalmic, pulmonary, intradermal, transdermal, transcutaneous or intradermal or by a combination of these routes. Routes of administration also include administration by inhalation or pulmonary aerosol. Techniques for preparing aerosol delivery systems are well known to those of skill in the art (see, for example, Sciarra and Cutie, “Aerosols,” in Remington's Pharmaceutical Sciences, 18th edition, 1990, pp. 1694-1712; incorporated by reference). In one embodiment, the TIPs of the present invention are administered in soluble form in the absence of adjuvant. In one embodiment, the TIPs are administered by a mucosal route. Studies have shown that peptide, when given in soluble form intraperitoneally (i.p.), intravenously (i.v.) or intranasally (i.n.) or orally can induce T cell tolerance (Anderton and Wraith (1998) as above; Liu and Wraith (1995) as above; Metzler and Wraith (1999) Immunology 97:257-263). In one embodiment, the TIP is administered intranasally.

Studies in mice have demonstrated that the duration of peptide administration required to induce tolerance depends on the precursor frequency of T cells in the recipient (Burkhart et al. (1999) as above). In many experimental studies, it has been shown that repeated doses of peptide are required to induce tolerance (Burkhart et al. (1999) as above). The exact dose and number of doses of TIP will therefore depend on the individual; however, in one embodiment a plurality of doses is administered.

If a plurality of TIPs or TIP sets is administered simultaneously, they may be in the form of a “cocktail” which is suitable for administration in single or multiple doses. Alternatively it may be given in multiple doses but vary the relative concentrations of the different TIPs between doses.

In some embodiments, the TIP compositions of the present invention are associated with, combined with, or administered with immunosuppressive compounds capable of inducing adaptive regulatory T cells. In one embodiment, the immunosuppressive compounds may include, but is not limited to, IL-10, TGF-β, and/or rapamycin and/or other limus compounds, including but not limited to biolimus A9, everolimus, tacrolimus, and zotarolimus, and/or combinations thereof. Methods for administering peptides in combination with immunosuppressive compounds are described, for example, in Nayak et al. Prevention and Reversal of Antibody Responses Against Factor IX Gene Therapy for Hemophilia B. Front Microbiol 2011; 2: 244.

In one embodiment a “dose escalation” protocol may be followed, where a plurality of doses is given to the patient in ascending concentrations. Such an approach has been used, for example, for phospholipase A2 peptides in immunotherapeutic applications against bee venom allergy (Müller et al. (1998) J. Allergy Clin Immunol. 101:747-754 and Akdis et al. (1998) J. Clin. Invest. 102:98-106).

In one aspect, the amount of TIPs to be administered may be determined using a stoichiometric calculation based on current ITI administration protocols. For example, the amount of a TIP to be administered are based on the equivalent quantity of the peptide that would be administered in a standard ITI protocol which uses the full length FVIIIrp. To determine dosing period, the subject's dendritic cells' reactivity to the TIPs is determined prior to the start of TIP administration, and then periodically monitored until tolerance to the TIPs is observed. For example, administration of the TIPs may occur over a 30 to 60 day period, wherein the subject's DC response to the TIPs are monitored (or, inhibitor concentration is monitored), and, when acceptable thresholds are reached, TIP administration ceases.

### EXAMPLES

In all examples of practicing a subject at risk of developing an anti-FVIII immune response or experiencing an anti-FVIII immune is administered one or more TIP(s) linked to a carrier.

### Example 1

### Treatment of a Subject Free of Anti-FVIII Antibodies

When a subject is in need of replacement FVIII therapy but has not yet received any replacement FVIII therapy or has received FVIII replacement but is free from anti-FVIII antibodies the following steps may be performed. One of ordinary skill in the art will appreciate that for such a subject it will be effective to administer TIPs linked to a carrier that incorporate any sequence differences between the sFVIII and rFVIIIrp (the amino acid reference locus or AARL in the context of 1-2332 possible positions for wild type FVIII).

Hemophilia Disease History and Clinical Characterization

A full hemophilia disease history of the patient is taken by a licensed physician using methods well established in the art (Robert A Zaiden, MD; Chief Editor: Steven C Dronen, MD, FAAEM. “Hemophilia A” Medscape Reference. Posting date: Dec. 23, 2013. Date material was accessed: Mar. 5, 2014. http://emedicine.medscape.com/article/779322). In addition clinical characterization of the patient's hemophilia disease is performed using laboratory tests to include measurement of hemoglobin/hematocrit, platelet count, measurement of prothrombin time, measurement of activated partial thromboplastin time (aPTT), and measurement of Factor (F)VIII activity by FVIII assay.

Sequence Patient's F8 Gene

During the development of the immune system in healthy humans, the cells and molecules of the immune system are instructed to be tolerant of self-proteins and other macromolecules that are produced endogenously, the result of which prevents the immune system from attacking self. When foreign proteins or other macromolecules are introduced into a healthy individual, the immune system recognizes these as foreign by default, as the immune system has been made tolerant only of self. Thus for many hemophilia patients, where a genetic lesion has caused the gene for FVIII to be altered in sequence, the FVIIIrp from healthy donors that is infused therapeutically may be seen as a foreign molecule. As a result the immune system mounts a response against the infused FVIIIrp, resulting in inhibitors. Importantly it is the residues or sequence of residues that differ between the patient's FVIII and the infused FVIIIrp that causes the initiation of the immune response. As a result, in order to provide therapy that leads to immune tolerance of the infused FVIIIrp, as outlined here, the sequence of the patient's FVIII gene (called F8) is compared to the sequence of the infused FVIIIrp to determine the location of residues that differ between the two. Using methods that are routine in the clinical laboratory (Viel, K. R. et al. Inhibitors of factor VIII in black patients with hemophilia. N Engl J Med 360, 1618-1627 (2009); Jacob, H. J. Next-generation sequencing for clinical diagnostics. N Engl J Med 369, 1557-1558 (2013); Yang, Y. et al. Clinical whole-exome sequencing for the diagnosis of mendelian disorders. N Engl J Med 369, 1502-1511 (2013)), the entirety of the patient's F8 gene will be sequenced. Using a routine computer software program (such as LALIGN, http://embnet.vital-it.ch/software/LALIGN_form.html) to align the sequence of the patient's F8 gene with the reference sequence from the infused FVIIIrp, four different parameters are assessed; for example (i) the causative mutation of hemophilia; (ii) the haplotype of the patient's F8 gene; (iii) other private non-synonymous single nucleotide polymorphisms (nsSNP) that are specific to the patient; (iv) differences between the patient's F8 gene and the FVIIIrp arising from engineered changes in the FVIIIrp deemed useful for facilitating expression, such as deletion of the B domain and insertion of a synthetic linker or to enhance half-life. A person of ordinary skill in the art can appreciate the numerous different computer software programs may be used for the alignment of protein sequences for the detection of differences between the patient's FVIII protein and that of FVIIIrp.

Assemble Information on Patient's F8 Mutation, Haplotype, and Private nsSNPs

The differences in protein sequence between the patient's FVIII and the FVIII replacement product were determined. These data are assembled for determining the TIPs that need to be prepared to induce immune tolerance to replacement FVIII in the patient.

Design TIPs Apropos to the Differences Between the Patient's FVIII and the FVIII Replacement Product

Using TIP design methods laid out in the detailed description above, pools of TIPs are designed for each of the protein sequence differences between the patient's FVIII and the replacement FVIII, For example, a pool of TIP of 15 amino acids in length are designed around each reference locus that arises from the difference in sequence between the patient's FVIII protein and the replacement FVIII protein. The number of TIP sequences in each pool of TIPs in this example is 15. The number of pools of TIPs equal to the number of differences in protein sequence between the patient's FVIII and the replacement FVIII.

Synthesize TIP Sets

TIPs are synthesized under good manufacturing practices (GMP). Numerous companies synthesize custom GMP-grade peptides in the range of 9-21 amino acids in length (for example AmbioPharm, Inc, http://www.ambiopharm.com). Upon transmitting to the manufacturer the sequences of TIPs required for treatment of the patient, the TIPs are synthesized and delivered.

Synthesize PLGA Nanoparticles

Numerous companies synthesize GMP-grade PLGA nanoparticles under highly defined specifications of size and surface chemistry (for example Phosphorex, Inc, http://www.phosphorex.com). Clinical-grade PLGA particles 500 nm in diameter with a surface chemistry containing carboxyl groups are obtained from a GMP-grade PLGA manufacturer.

Conjugate TIPs to PLGA Nanoparticles

Conjugating peptides such as TIPs described herein to carboxylated PLGA particles is a method well established in the art and routinely performed by persons of ordinary skill in the art (Getts, D. R. et al. Microparticles bearing encephalitogenic peptides induce T-cell tolerance and ameliorate experimental autoimmune encephalomyelitis. Nat Biotechnol 30, 1217-1224 (2012)). In the presence of EDC (1-ethyl-3-[3-dimethylaminopropyl]carbodiimide), the carboxyl moieties on the surface of carboxylated PLGA particles react to form a covalent bond with the terminal primary amine group present in all TIPs. This results in the formation of an amide bond between the PLGA particles and TIP. The TIP pool synthesized above are mixed together with the 500 nm carboxylated PLGA particles in the presence of EDC at a ratio of 0.08 mg of each TIP to 1.0 mg PLGA particles to 0.32 mg EDC in buffered aqueous solution. The coupling process is performed for each TIPs set. Following the conjugation reaction the buffered aqueous solution is exchanged a minimum of three times. It is appreciated by persons of ordinary skill in the art that other ratios of TIP to PLGA particle to EDC may be used for this procedure. It is appreciated by persons of ordinary skill in the art that PLGA particles of sizes greater than or small than 500 nm in diameter may be used for this procedure. It is appreciated by persons of ordinary skill in the art that carriers other than PLGA may be used for conjugation to TIP. It is appreciated by persons of ordinary skill in the art that chemical formulations other than EDC may be used for conjugating TIP to carriers.

Quality Control for TIP-Nanoparticle Sets

Using methods well established in the art and routinely performed by persons of ordinary skill in the art (Lutterotti, A. et al. Antigen-specific tolerance by autologous myelin peptide-coupled cells: a phase 1 trial in multiple sclerosis. Sci Transl Med 5, 188ra75 (2013)), the following quality control measures will be taken for the PLGA-TIP conjugates: (1) Verification of coupling of the TIP to PLGA particles by flow cytometry; (2) Analysis of the conjugation product to verify that residual EDC is at a concentration less than 1.9 μg/mL; (3) Analysis of the conjugation product to verify that the concentration of endotoxin is less than 0.5 endotoxin units/mL; and (4) Analysis of the conjugation product to verify that the pH is greater than or equal to 7.2 and less than or equal to 7.8.

Administer TIP-Nanoparticles to Patient by Intravenous Injection

The PLGA-TIP particles that meet the quality control parameters above are suspended in pharmaceutical grade saline to a concentration of 5×10¹⁰ particles/mL. It is appreciated by persons of ordinary skill in the art that PLGA-TIP concentrations greater than 5×10¹⁰ may be used. It is appreciated by persons of ordinary skill in the art that PLGA-TIP concentrations less than 5×10¹⁰ may be used. For each TIP set, 3.5×10¹⁰ particles per kilogram weight of the patient are injected intravenously into the patient by a licensed physician using standard clinical practices. It is appreciated by persons of ordinary skill in the art that doses greater than 3.5×10¹⁰ particles per kilogram weight of the patient may be used. It is appreciated by persons of ordinary skill in the art that doses less than 3.5×10¹⁰ particles per kilogram weight of the patient may be used.

Physical Examination and Laboratory Tests are performed after the administration of TIP nanoparticles to obtain data of blood count, chemistry panel, urinalysis, and a lipid panel.

Updated Hemophilia Disease History and Clinical Characterization

A follow-up hemophilia disease history of the patient is taken by a licensed physician. In addition clinical characterization of the patient's hemophilia disease is performed using laboratory tests to include by not limited to measurement of hemoglobin/hematocrit, platelet count, measurement of prothrombin time, measurement of activated partial thromboplastin time (aPTT), and measurement of Factor (F)VIII activity by FVIII assay.

### Example 2

### Treatment and Monitoring of Immune Response in a Subject Free of Anti-FVIII Antibodies at Onset of TIP Tolerance Induction Therapy

In the case of subject who is free of neutralizing FVIII antibodies at the onset of a tolerance induction therapy it may be useful to do all of the steps done in Example 1 and, in addition monitor the subject's immune response to putative T cell epitopes in the FVIIIrp identified by sequence analyses as described in Example 1 and the immune response to FVIIIrp

Ex Vivo T Cell Assay Using TIPs as Target Antigen

The presence and abundance of circulating effector T cells are measured in samples obtained from the patient. Antigen-specific lymphoproliferative assays are used to test for the presence in the patient's peripheral blood of T cells that recognize and respond to FVIII TIPs. Cells are labeled with the fluorescent dye 5,6-carboxyfluorescein diacetate succinimidyl ester (CFSE). Those cells that proliferate in response to antigen show a reduction in CFSE fluorescence intensity, which is measured directly by flow cytometry. Since this is a flow cytometric assay, it accurately determines the percentage of proliferating CD4+ cells, enables detailed phenotyping of T cell responses, and is more sensitive than traditional assays based on radioactive thymidine incorporation. ELISA assays are used to measure bulk secretion of cytokines produced by FVIII antigen-specific T cells derived from the patient's peripheral blood. ELISpot assays are used to enumerate the number of cytokine-secreting FVIII-specific T cells derived from the patient's peripheral blood. These assays may be repeated periodically until the subject has received 50 or more infusions on FVIIIrp

Determine Inhibitor Titer

In order to monitor the efficacy of treatment with a TIP protocol, an initial measure of the severity of the patient's FVIII inhibitor problem (if any), with subsequent measurements are taken subsequent to treatment to monitor the effect of the treatment on the patient's inhibitors. To determine the patient's titer of FVIII inhibitory antibodies, two methods are used, both of which are standard assays in medical diagnostics and are well known in the art (Peerschke, E. I. B. et al. Laboratory assessment of factor VIII inhibitor titer: the North American Specialized Coagulation Laboratory Association experience. Am J Clin Pathol 131, 552-558 (2009)). Firstly, a Bethesda assay using the Nijmegen modification is performed. This assay yields a measure of inhibitor titer in the form of Bethesda Units per milliliter of patient plasma (BU/mL). A titer of 1-5 BU/mL is considered mild for inhibitors, while a titer of >5 BU/mL is considered severe. This assay has the advantage of directly measuring the inhibition of FVIII activity by inhibitors, but has the limitation that it is less sensitive when inhibitor titers are low (0-1 BU/mL). Secondly, an enzyme-linked immunosorbant assay (ELISA) is performed. This assay measures the total amount of antibodies that are specific for FVIII in the patient's plasma, including inhibitory antibodies. This assay has the advantages of being highly sensitive, of determining the isotype of the anti-FVIII antibodies, and of measuring both inhibitory and non-inhibitory anti-FVIII antibodies. It has the limitation of not directly measuring the titer of inhibitory antibodies alone. Taken together, these two assays give a nearly complete view of the antibody immune response against FVIII.

Quantitate FVIII-Reactive B Cells by ELISpot Assay

As another parameter to measure the immune response against FVIII and the efficacy of treatment, the number of circulating FVIII-specific antibody-secreting B cells in the patient's peripheral blood are measured. The enzyme-linked immunosorbant spot (ELISpot) assay is a common immunological tool used by persons of ordinary skill in the art; which tool facilitates measurement of the number of antigen-specific B cells in peripheral blood (Czerkinsky, C. C., et al. A solid-phase enzyme-linked immunospot (ELISPOT) assay for enumeration of specific antibody-secreting cells. J Immunol Methods 65:109-121 (1983); Bondada, S. & Robertson, D. A. Assays for B lymphocyte function. Curr Protoc Immunol Chapter 3, Unit 3.8 (2003)). Using purified human FVIII as the target antigen to coat PVDF membranes of ELISpot microtiter plates, the number of B cells that secrete antibody specific for FVIII is quantitated from the buffy coat of a peripheral blood draw using established methods. This assay yields as a result the number of FVIII-specific B cells in peripheral blood, expressed in units as number of cells per milliliter of blood (#/mL). The values obtained by this assay prior to treatment are used as reference for subsequent assays that are performed post-treatment, as detailed below.

Regulatory T Cell Assay Using FVIII and/or TIPs as Target Antigen

The presence and abundance of circulating regulatory T cells are measured in samples obtained from the patient. White blood cells from the peripheral blood of patients are isolated to test for the presence and abundance of regulatory T cells specific for FVIII and/or FVIII TIPs.

### Example 3

### Treatment and Monitoring of Immune Response in a Subject with Neutralizing Anti-FVIII Antibodies at Onset of TIP Tolerance Induction Therapy

In the case of subject who has high titer neutralizing FVIII antibodies at the onset of a TIP tolerance induction therapy it may be useful to do all of the steps done in Example 1 and 2. However it would also be useful to administer TIPs that help induce tolerance any T cell in the FVIIIrp; not only those T cell epitopes that may arise when regions of the FVIIIrp that harbour an AARL are liberated by the subject's immune system.

Bioinformatics to Assist in the Design of TIPs for Tolerizing a Subject to an Array of T Cell Epitopes in FVIIIrp.

For example, Next Generation Sequencing technology is used to determine the complete set of HLA genes for a subject with an established high titer anti-FVIII immune response. Children's Hospital of Philadelphia offers this service. It is possible to use in silico methods to evaluate which peptides regions within an FVIIIrp are likely to bind the subject's MCH II proteins with adequate affinity and stability to initiate an immune response. One or more sets of such candidate T cell epitopes/peptides are evaluated in the ex vivo T cell assay described in example 2 using the peptides as target antigens. Peptides that trigger T cell proliferation are used to derive TIPs coupled to carriers for administration to the subject.

Ex Vivo T Cell Assay Using FVIIIrp as the Target Antigen

Proimmune has developed a DC-T cell assay that is useful for identifying T cell epitopes in replacement protein products such as FVIIIrp. Fully-formulated proteins are used in the assay. For example, donor PBMC are used as a source of monocytes that are cultured in defined media to generate immature dendritic cells. Dendritic cells are loaded with test antigen (whole protein), and are then induced into a more mature phenotype by further culture in defined media. CD8+ T cell-depleted donor PBMC from the same donor sample are labeled with CFSE then cultured with the antigen-primed DCs for 7 days, after which octuplicates are tested. Each DC-T cell culture includes a set of untreated control wells. The assay also incorporates reference antigen controls, comprising two potent whole protein antigens. This assay is customized to incorporate a subject's PBMCs and the replacement FVIIIrp to monitor the progress and maintenance of tolerance in a subject. Other methods may be used to monitor the presence in peripheral blood of effector T cells that are specific for FVIII as an indicia of ongoing immunity against the antigen. One expects in a patient with FVIII inhibitory antibodies that these effector T cells will be present. In contrast, in patients that have either no FVIII inhibitor antibodies or in patients that had FVIII inhibitory antibodies and have been subsequently immune tolerized to FVIII, one expects the absence or near absence of these cells in peripheral blood. As another parameter for measuring the immune response of patients against FVIII, the abundance and phenotype of these cells are measured in the peripheral blood of patients. Several methods are well established in the art and commonly employed by persons of ordinary skill in the art for measuring the abundance and phenotype of effector T cells in peripheral human blood (Clay, T. M., et al. Assays for monitoring cellular immune responses to active immunotherapy of cancer. Clin Cancer Res 7, 1127-1135 (2001); Kruisbeek, A. M., Shevach, E. & Thornton, A. M. Proliferative assays for T cell function. Curr Protoc Immunol Chapter 3, Unit 3.12 (2004); Mannering, S. I. et al. Current approaches to measuring human islet-antigen specific T cell function in type 1 diabetes. Clin Exp Immunol 162, 197-209 (2010)). Antigen-specific lymphoproliferative assays are used to test for the presence in the patient's peripheral blood of T cells that recognize and respond to FVIIIrp protein and/or to TIPs described herein. This method additionally allows the characterization of the phenotype of the T cells that respond to the FVIII antigen and/or TIPs, including but not limited to the cytokines produced by the cells, and the polarization of the T cells into T cell lineages, including but not limited to T-helper-1 cells, T-helper-2 cells, and T-helper-17 cells. ELISA assays are used to measure bulk secretion of cytokines produced by FVIII antigen-specific T cells derived from the patient's peripheral blood. ELISpot assays are used to enumerate the number of cytokine-secreting FVIII-specific T cells derived from the patient's peripheral blood

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein.

1.-10. (canceled)

11. A method of providing a tolerance inducing peptide (TIP), the method comprising,
determining an amino acid reference locus (AARL) within a FVIII replacement product (FVIIIrp) by determining differences between protein sequences of an expression product of a subject's F8 gene (sFVIII) and the FVIIIrp, and
providing a TIP comprising the AARL of the FVIIIrp, amino acid residues upstream of the AARL corresponding with contiguous amino acid residues upstream of the AARL in the FVIIIrp and/or amino acid residues downstream from the AARL corresponding with contiguous amino acid residues downstream of the AARL FVIIIrp.

12. (canceled)

13. The method of claim 11, wherein the TIP has a length of X amino acid residues corresponding with a contiguous portion of the FVIIIrp across 2X−1 amino acids including X−1 amino acid residues upstream and X−1 amino acid residues downstream from an amino acid of the AARL within the FVIIIrp.

14. The method of claim 13, wherein providing a TIP is performed by providing a set of TIPs, with each TIP within the set of TIPs having a length of X unique amino acid residues and a first amino acid residue shifted one residue upstream in the FVIIIrp sequence with reference to the AARL and wherein the set of TIPs collectively overlaps a contiguous portion of the FVIIIrp sequence spanning a length of 2X−1 residues.

15.-16. (canceled)

17. A composition comprising a TIP prepared in accordance with the method of claim 16.

18. The composition of claim 17, wherein the TIP has a sequence selected from the group consisting of SEQ. ID No.14 to SEQ. ID No. 2438.

19. The composition of claim 17, wherein the TIP is linked to a carrier.

20. The composition of claim 19, wherein the carrier is a poly(lactide-co-glycolide)(PLGA) particle or a poly(lactide-co-glycolide)(PLGA) particle modified with PEMA (poly[ethyleneco-maleic acid]) as a surfactant as a PLGA-PEMA particle having a size of between about 10 nm to about 5000 nm.

21-38. (canceled)

39. A method of inducing tolerance to an FVIII replacement product (FVIIIrp) in a subject, the method comprising
administering to the subject at least one tolerance inducing peptide (TIP) comprising the AARL of the FVIIIrp, amino acid residues upstream of the AARL corresponding with contiguous amino acid residues upstream of the AARL in the FVIIIrp and/or amino acid residues downstream from the AARL corresponding with contiguous amino acid residues downstream of the AARL FVIIIrp
the at least one tolerance inducing peptide administered to the subject in an effective amount to induce tolerance or reduce or minimize an immune response to the FVIII replacement product.

40. The method of claim 39, wherein the administering is performed prior to the development of inhibitors to the FVIIIrp in the subject.

41. The method of claim 39, wherein the subject has inhibitors to the FVIIIrp and the administering results in at least 20% reduction of measurable Bethesda titer units to the FVIIIrp in the subject.

42. The method of claim 39, wherein the administering is performed by administering the at least one TIP in addition to other FVIII tolerance induction therapy.

43. The method of claim 39, wherein the at least one TIP induces T cell proliferation in a T cell lymphoproliferation assay.

44. The method of claim 39, further comprising detecting the subject's immune response via Bethesda or FVIII reactive B cell assay prior to the administering of and intermittently following the administering during the course of therapy.
